(19) United States
(12) Patent Application Publication
(10) Pub. No.: US 2021/0323913 A1
(43) Pub. Date: Oct. 21, 2021

(54) SIGMA RECEPTOR BINDERS

(71) Applicant: BOARD OF REGENTS, THE UNIVERSITY OF TEXAS SYSTEM, Austin, TX (US)

(72) Inventors: Stephen F. MARTIN, Austin, TX (US); James J. SAHN, Austin, TX (US); Michael Dean WOOD, Austin, TX (US)

(21) Appl. No.: 16/097,171

(22) PCT Filed: Apr. 29, 2017
(86) PCT No.: PCT/US2017/030296

§ 371 Date: Oct. 26, 2018

Related U.S. Application Data
(60) Provisional application No. 62/329,864, filed on Apr. 29, 2016.

(51) Int. Cl. C07C 271/24; C07D 207/27; C07D 233/61; C07D 295/096

(52) U.S. Cl. CPC C07C 271/24; C07D 295/096; C07D 233/61; C07D 207/27

(57) ABSTRACT

Provided herein, inter alia, are compounds and methods of treating diseases including cancer, neurological disease, alcohol withdrawal, depression and anxiety, traumatic brain injury, and neuropathic pain.

### CROSS-REFERENCES TO RELATED APPLICATIONS

This application is a national phase application under 35 U.S.C. § 371 of International Application No. PCT/US2017/030296, filed Apr. 29, 2017, which claims the benefit of priority to U.S. Provisional Application No. 62/329,864, filed on Apr. 29, 2016, the entire contents of each of which are hereby incorporated by reference.

### BACKGROUND OF THE INVENTION

Alzheimer's Disease is one of the most common dementia among older adults. As many as 5.3 million people in the United States are living with Alzheimer's, with that number expected to grow to 14 million by 2050. ALS is one of the most common neuromuscular diseases for which there is currently no cure.

Cancer is a leading cause of death around the world, according to the World Health Organization. Cases of cancer doubled globally between 1975 and 2000, will double again by 2020, and will nearly triple by 2030. There were an estimated 12 million new cancer diagnoses and more than seven million deaths worldwide this year.

Substance abuse is a significant health problem in the USA, as well as in other countries, and is estimated to cost society over 1 billion dollars per year. There are currently very limited pharmacotherapies to treat substance abuse.

Sigma receptors are transmembrane proteins expressed in many tissues and have been implicated in, for example, cardiovascular function, substance abuse, and cancer. Many known sigma receptor ligands lack either sigma subtype selectivity or general selectivity.

It is desirable to have new therapeutics effective at treating these diseases. Provided herein are solutions to these and other problems in the art.

### BRIEF SUMMARY OF THE INVENTION

Provided herein are compositions and methods useful as pharmaceutical agents. In one aspect is a compound having the formula:

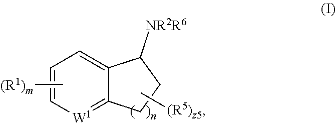

R¹ is halogen, —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups; R² is halogen, —N₃, —CF₃, —CCl₃, —CBr₃, —CI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙ₂NR⁴R^{4A}, —NHNR⁴R^{4A}, —ONR⁴R^{4A}, —NHC(O)NHNR⁴R^{4A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups; n1 and n2 are independently 1 or 2; m is 1, 2, 3 or 4; n is 1, 2, 3 or 4; R³, R^{3A}, R⁴, R^{4A} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups; R⁵ is halogen, —N₃, —CF₃, —CCl₃, —CBr₃, —CI₃, —CN, —C(O)R^{5C}, —OR^{5D}, —NR^{5A}R^{5B}, —C(O)OR^{5D}, —C(O)NR^{5A}R^{5B}, —NO₂, —SR^{5D}, —S(O)ₙ₅R^{5C}, —S(O)ₙ₅OR^{5D}, —S(O)ₙ₅NR^{5A}R^{5B}, —NHNR^{5A}R^{5B}, —ONR^{5A}R^{5B}, —NHC(O)NHNR^{5A}R^{5B}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups; n5 is independently 1 or 2; z5 is independently and integer from 0 to 6; R⁶ is halogen, —N₃, —CF₃, —CCl₃, —CBr₃, —CI₃, —CN, —C(O)R^{6C}, —OR^{6D}, —NR^{6A}R^{6B}, —C(O)OR^{6D}, —C(O)NR^{6A}R^{6B}, —NO₂, —SR^{6D}, —S(O)ₙ₆R^{6C}, —S(O)ₙ₆OR^{6D}, —S(O)ₙ₆NR^{6A}R^{6B}, —NHNR^{6A}R^{6B}, —ONR^{6A}R^{6B}, —NHC(O)NHNR^{6A}R^{6B}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups; n6 is independently 1 or 2; W₁ is CH, C(R¹), or N; and R^{5A}, R^{5B}, R^{5C}, R^{5D}, R^{6A}, R^{6B}, R^{6C}, and R^{6D} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups. In embodiments, when R¹ is N-methylpiperazinyl, m is 1, W¹ is CH, n is 2, then R² is not benzyl.

Provided herein are pharmaceutical compositions. In one aspect is a pharmaceutical composition that includes a compound described herein, a pharmaceutically acceptable excipient, and a pharmaceutically acceptable salt.

Also provided here are methods of treating a disease. In one aspect is a method of treating cancer in a subject in need thereof by administering an effective amount of a compound described herein. In another aspect is a method of treating neurodegenerative disease in a subject in need thereof by administering an effective amount of a compound described herein. In still another aspect is a method of treating ethanol withdrawal in a subject in need thereof by administering an effective amount of a compound described herein. In yet another aspect is a method of treating anxiety or depression in a subject in need thereof by administering an effective amount of a compound described herein. In still yet another aspect is a method of treating neuropathic pain in a subject in need thereof by administering an effective amount of a compound described herein.

Further provided herein are methods of inhibiting or antagonizing a sigma 1 or sigma 2 receptor. In one aspect is a method of inhibiting/antagonizing a sigma 2 receptor by contacting a sigma 2 receptor with a compound described herein, thereby inhibiting the sigma 2 receptor. In another aspect is a method of inhibiting a sigma 1 receptor by contacting a sigma 1 receptor with a compound described herein, thereby inhibiting said sigma 1 receptor.

Provided herein are methods of activating or agonizing a sigma 1 or sigma 2 receptor. In one aspect is a method of activating/agonizing a sigma 2 receptor by contacting a sigma 2 receptor with a compound described herein, thereby activating the sigma 2 receptor. In another aspect is a method of activating a sigma 1 receptor by contacting a sigma 1 receptor with a compound described herein, thereby activating the sigma 1 receptor.

### DETAILED DESCRIPTION OF THE INVENTION

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. If used in the context of a larger list of chemical groups wherein unsaturated alkyl groups are specifically defined then the term “alkyl” is used to describe a saturated group. An unsaturated alkyl group may be further refined as alkenyl which is an unsaturated alkyl group with one or more carbon-carbon double bonds and no carbon-carbon triple bonds. Similarly, an unsaturated alkyl group may be further refined as alkynyl which is an unsaturated alkyl group with one or more carbon-carbon triple bonds. An alkynyl group may contain one or more carbon-carbon double bonds so long as it contains at least one carbon-carbon triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). Similarly, an aralkyl group is a substituted alkyl group which has been substituted with one or more aryl groups as this term is described herein. These aralkyl group may be substituted as described below in agreement with the common chemical bonding valency. Some non-limiting examples of unsubstituted aralkyl groups include benzyl, phenylethyl, and diphenylethyl. Furthermore, an aralkenyl group is a subset wherein the substituted alkyl group is an alkenyl group as that term has been defined above.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., selected from the group consisting of O, N, P, Si, and S, and wherein the nitrogen and sulfur atoms may optionally be oxidized, and the nitrogen heteroatom may optionally be quaternized). The heteroatom(s) (e.g., O, N, P, S, B, As, and Si) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. These groups include the possibility that one or more of these groups may have one or more saturated alkyl substitutions on the ring system provided that the point of connection is the ring system.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be a —O-bonded to a ring heteroatom nitrogen.

A “fused ring aryl-heterocycloalkyl” is an aryl fused to a heterocycloalkyl. A “fused ring heteroaryl-heterocycloalkyl” is a heteroaryl fused to a heterocycloalkyl. A “fused ring heterocycloalkyl-cycloalkyl” is a heterocycloalkyl fused to a cycloalkyl. A “fused ring heterocycloalkyl-heterocycloalkyl” is a heterocycloalkyl fused to another heterocycloalkyl. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be unsubstituted or substituted with one or more of the substituents described herein. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be named according to the size of each of the fused rings. Thus, for example, 6,5 aryl-heterocycloalkyl fused ring describes a 6 membered aryl moiety fused to a 5 membered heterocycloalkyl. Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “oxo,” as used herein, means an oxygen that is double bonded to a carbon atom.

The term “thio,” as used herein, means a sulfur that is single or double bonded to carbon, or single bonded to another sulfur.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, a substitutent group as that term is defined below or —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present. In some embodiments, the substitution may include the removal of one or more hydrogen atom and replacing it with one of the following groups: —OH, —F, —Cl, —Br, —I, —NH₂, —NO₂, —CO₂H, —CO₂CH₃, —CO₂CH₂CH₃, —CN, —SH, —OCH₃, —OCF₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —NHC(O)NH₂, —S(O)₂OH, —S(O)₂CH₃, or —S(O)₂NH₂.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), Boron (B), Arsenic (As), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC(O)NHNH₂, —NHC(O) NH₂, —NHSO₂H, —NHC(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

Unless otherwise defined herein, the chemical groups used herein may contain between 1 to 20 carbon atoms or ring members. In some preferred embodiments, the chemical group contains 1 to 12 carbon atoms or ring members. In more preferred embodiments, the chemical group contains 1 to 8 carbon atoms or ring members.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl, alkenyl, alkynyl, aryl, aralkyl, or aralkenyl may be a substituted or unsubstituted C₁-C₂₀ alkyl, alkenyl, alkynyl, aryl, aralkyl, or aralkenyl each substituted or unsubstituted heteroalkyl, heteroaryl, heteroaralkyl, or heteroaralkenyl is a substituted or unsubstituted 2 to 20 membered heteroalkyl, heteroaryl, heteroaralkyl, or heteroaralkenyl, each substituted or unsubstituted cycloalkyl or cycloalkenyl is a substituted or unsubstituted C₃-C₈ cycloalkyl or cycloalkenyl, and/or each substituted or unsubstituted heterocycloalkyl is a substituted or unsubstituted 3 to 8 membered heterocycloalkyl. In some embodiments of the compounds herein, each substituted or unsubstituted alkylene, alkenylene, alkynylene, arylene, aralkylene, or aralkenylene is a substituted or unsubstituted C₁-C₂₀ alkylene, alkenylene, alkynylene, arylene, aralkylene, or aralkenylene, each substituted or unsubstituted heteroalkylene, heteroarylene, heteroaralkylene, or heteroaralkenylene is a substituted or unsubstituted 2 to 20 membered heteroalkylene, heteroarylene, heteroaralkylene, or heteroaralkenylene, each substituted or unsubstituted cycloalkylene or cycloalkenylene is a substituted or unsubstituted C₃-C₈ cycloalkylene or cycloalkenylene, and/or each substituted or unsubstituted heterocycloalkylene is a substituted or unsubstituted 3 to 8 membered heterocycloalkylene.

In some embodiments, each substituted or unsubstituted alkyl, alkenyl, alkynyl, aryl, aralkyl, or aralkenyl is a substituted or unsubstituted C₁-C₈ alkyl, alkenyl, alkynyl, aryl, aralkyl, or aralkenyl, each substituted or unsubstituted heteroalkyl, heteroaryl, heteroaralkyl, or heteroaralkenyl is a substituted or unsubstituted 2 to 8 membered heteroalkyl, heteroaryl, heteroaralkyl, or heteroaralkenyl each substituted or unsubstituted cycloalkyl or cycloalkenyl is a substituted or unsubstituted C₃-C₇ cycloalkyl or cycloalkenyl, and/or each substituted or unsubstituted heterocycloalkyl is a substituted or unsubstituted 3 to 7 membered heterocycloalkyl. In some embodiments, each substituted or unsubstituted alkylene, alkenylene, alkynylene, arylene, aralkylene, or aralkenylene is a substituted or unsubstituted C₁-C₈ alkylene, alkenylene, alkynylene, arylene, aralkylene, or aralkenylene, each substituted or unsubstituted heteroalkylene, heteroarylene, heteroaralkylene, or heteroaralkenylene is a substituted or unsubstituted 2 to 8 membered heteroalkylene, heteroarylene, heteroaralkylene, or heteroaralkenylene, each substituted or unsubstituted cycloalkylene or cycloalkenylene is a substituted or unsubstituted C₃-C₇ cycloalkylene or cycloalkenylene, and/or each substituted or unsubstituted heterocycloalkylene is a substituted or unsubstituted 3 to 7 membered heterocycloalkylene.

Certain compounds of the present invention possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds, generally recognized as stable by those skilled in the art, are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The symbol “-” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a decimal symbol may be used to distinguish each appearance of that particular R group. For example, where multiple R¹³ substituents are present, each R¹³ substituent may be distinguished as R^{13.1}, R^{13.2}, R^{13.3}, R^{13.4}, etc., wherein each of R^{13.1}, R^{13.2}, R^{13.3}, R^{13.4}, etc. is defined within the scope of the definition of R¹³ and optionally differently.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein include those compounds that readily undergo chemical or enzymatic changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease.

An “effective amount” is an amount sufficient to accomplish a stated purpose (e.g. achieve the effect for which it is administered, treat a disease, reduce enzyme activity, increase enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In embodiments, a control is the measurement of the activity of a protein in the absence of a compound as described herein (including embodiments and examples).

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “inhibition”, “inhibit”, “inhibiting”, and “antagonizing” the like in reference to a protein-inhibitor interaction means negatively affecting (e.g. decreasing) the activity or function of the protein relative to the activity or function of the protein in the absence of the inhibitor. In embodiments inhibition refers to reduction of a disease or symptoms of disease. In embodiments, inhibition refers to a reduction in the activity of a particular protein or nucleic acid target. Thus, inhibition includes, at least in part, partially or totally blocking stimulation, decreasing, preventing, or delaying activation, or inactivating, desensitizing, or down-regulating signal transduction or enzymatic activity or the amount of a protein.

The terms “activation”, “activate”, “activating”, and “agonizing” and the like refer to positively affecting (e.g. increasing) the activity or function of the protein relative to the activity or function of the protein in the absence of the activator. Activation may include, at least in part, partially or totally increasing stimulation, increasing or enabling activation, or activating, sensitizing, or up-regulating signal transduction or enzymatic activity or the amount of a protein decreased in a disease. Activation may include, at least in part, partially or totally increasing stimulation, increasing or enabling activation, or activating, sensitizing, or up-regulating signal transduction or enzymatic activity or the amount of a protein that may modulate the level of another protein or increase cell survival.

The term “modulator” refers to a composition that increases or decreases the level of a target molecule or the function of a target molecule or the physical state of the target of the molecule.

The term “modulate” is used in accordance with its plain and ordinary meaning and refers to the act of changing or varying one or more properties. “Modulation” refers to the process of changing or varying one or more properties. For example, a modulator of a target protein changes by increasing or decreasing a property or function of the target molecule or the amount of the target molecule. A modulator of a disease decreases a symptom, cause, or characteristic of the targeted disease.

“Selective” or “selectivity” or the like of a compound refers to the compound's ability to discriminate between molecular targets. “Specific”, “specifically”, “specificity”, or the like of a compound refers to the compound's ability to cause a particular action, such as inhibition, to a particular molecular target with minimal or no action to other proteins in the cell.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The compositions disclosed herein can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions disclosed herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). The formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions can also be delivered as nanoparticles.

Pharmaceutical compositions may include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

The compounds and complexes described herein can be used in combination with one another, with other active drugs known to be useful in treating a disease (e.g. anti-cancer drugs) or with adjunctive agents that may not be effective alone, but may contribute to the efficacy of the active agent.

By “co-administer” it is meant that a composition described herein is administered at the same time, just prior to, or just after the administration of one or more additional therapies, for example an anticancer agent as described herein. The compound of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g. anticancer agents).

Co-administration includes administering one active agent (e.g. a compound described herein or an anti-cancer agent) within 0.5, 1, 2, 4, 6, 8, 10, 12, 16, 20, or 24 hours of a second active agent (e.g. a compound described herein or an anti-cancer agent). Also contemplated herein, are embodiments, where co-administration includes administering one active agent (e.g. a compound herein) within 0.5, 1, 2, 4, 6, 8, 10, 12, 16, 20, or 24 hours of a second active agent (e.g. a compound described herein or an anti-cancer agent). Co-administration includes administering two active agents simultaneously, approximately simultaneously (e.g., within about 1, 5, 10, 15, 20, or 30 minutes of each other), or sequentially in any order. Co-administration can be accomplished by co-formulation, i.e., preparing a single pharmaceutical composition including both active agents. The active agents can be formulated separately. The active and/or adjunctive agents may be linked or conjugated to one another. The compounds and complexes described herein may be combined with treatments for cancer, when administered to a subject in need thereof, such as chemotherapy or radiation therapy.

The term “associated” or “associated with” in the context of a substance or substance activity or function associated with a disease means that the disease is caused by (in whole or in part), a symptom of the disease is caused by (in whole or in part) the substance or substance activity or function, or a side-effect of the compound (e.g. toxicity) is caused by (in whole or in part) the substance or substance activity or function.

“Patient,” “subject,” “patient in need thereof,” and “subject in need thereof” are herein used interchangeably and refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. A patient may be human.

“Disease” or “condition” refer to a state of being or health status of a patient or subject capable of being treated with the compounds or methods provided herein. Disease as used herein may refer to cancer, a neurodegenerative disease, alcohol withdrawal, depression, anxiety, or neuropathic pain.

As used herein, the term “neurodegenerative disease” refers to a disease or condition in which the function of a subject's nervous system becomes impaired. Examples of neurodegenerative diseases that may be treated with a compound or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, drug-induced Parkinsonism, progressive supranuclear palsy, corticobasal degeneration, multiple system atrophy, Idiopathic Parkinson's disease, Autosomal dominant Parkinson disease, Parkinson disease, familial, type 1 (PARK1), Parkinson disease 3, autosomal dominant Lewy body (PARK3), Parkinson disease 4, autosomal dominant Lewy body (PARK4), Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK8), Parkinson disease 9 (PARK9), Parkinson disease 10 (PARK10), Parkinson disease 11 (PARK11), Parkinson disease 12 (PARK12), Parkinson disease 13 (PARK13), or Mitochondrial Parkinson's disease. Neurological disease as used herein may refer to Alzheimer's disease or ALS.

As used herein, the term “cancer” refers to all types of cancer, neoplasm, or malignant or benign tumors found in mammals, including leukemia, carcinomas and sarcomas. Exemplary cancers include acute myeloid leukemia (“AML”), chronic myelogenous leukemia (“CML”), and cancer of the brain, breast, triple-negative breast cancer, pancreas, colon, liver, kidney, lung, non-small cell lung, melanoma, ovary, sarcoma, and prostate. Additional examples include, cervix cancers, stomach cancers, head & neck cancers, uterus cancers, mesothelioma, metastatic bone cancer, Medulloblastoma, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, and neoplasms of the endocrine and exocrine pancreas.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The murine leukemia model is widely accepted as being predictive of in vivo anti-leukemic activity. It is believed that a compound that tests positive in the P388 cell assay will generally exhibit some level of anti-leukemic activity regardless of the type of leukemia being treated. Accordingly, the present invention includes a method of treating leukemia, including treating acute myeloid leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with a combination of antineoplastic thiol-binding mitochondrial oxidant and an anticancer agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with a combination of antineoplastic thiol-binding mitochondrial oxidant and an anticancer agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with a combination of antineoplastic thiol-binding mitochondrial oxidant and an anticancer agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

“Anti-cancer agent” is used in accordance with its plain and ordinary meaning and refers to a composition (e.g. compound, drug, antagonist, inhibitor, modulator) having antineoplastic properties or the ability to inhibit the growth or proliferation of cells. An anti-cancer agent may be a chemotherapeutic agent. An anti-cancer agent may be an agent approved by the FDA or similar regulatory agency of a country other than the USA, for treating cancer.

Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rIL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Gudrin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™) afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

The terms “Chemotherapeutic” and “chemotherapeutic agent” are used in accordance with their plain and ordinary meaning and refer to a chemical composition or compound having antineoplastic properties or the ability to inhibit the growth or proliferation of cells.

“Cancer model organism”, as used herein, is an organism exhibiting a phenotype indicative of cancer, or the activity of cancer causing elements, within the organism. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

The terms “ethanol withdrawal,” “alcohol withdrawal,” and “alcohol withdrawal syndrome” are used interchangeably herein and refer to diseases associated with and/or symptoms associated cessation of prolonged or excessive alcohol drinking. Symptoms may include, but are not limited to, anxiety, irritability, agitations, tremors, seizures, confusion, tachycardia, and infections.

“Neuropathic pain” is used according to its plain and ordinary meaning and refers to pain, both episodic and chronic, associated with nerve fiber damage, dysfunction, or injury.

The term “traumatic brain injury” or “TBI” is used according to its plain and ordinary meaning and refers to the resultant injury to nerves or the brain caused by an external force. TBI can result in physical, cognitive, social, emotional, and behavioral symptoms and can results in an injury which results in full recovery or permanent disability or damage including death. Even after the initial event, a secondary injury is included in the term traumatic brain injury wherein the cerebral blood flow or pressure within the skulls causes some damage to the brain itself. Additional events which are related to the secondary injury include damage to the blood-brain barrier, release of factors that cause inflammation, free radical overload, excessive release of the neurotransmitter glutamate (excitotoxicity), influx of calcium and sodium ions into neurons, dysfunction of mitochondria, damage to the white matter which results in the separate of cell bodies, changes in the blood flow to the brain; ischemia (insufficient blood flow); cerebral hypoxia (insufficient oxygen in the brain), cerebral edema (swelling of the brain), and raised intracranial pressure (the pressure within the skull). The primary injury results from the initial impact and includes damage from the trauma when tissues and blood vessels are stretched, compressed, and torn.

The terms “depression” and “anxiety” are used according to their ordinary and common meanings.

The term “sigma 1 receptor” is used according to its ordinary meaning in the art and refers to a transmembrane protein capable of modulating release of calcium and neurotransmitter systems. A sigma 1 receptor may be expressed in different tissues, and may be concentrated in areas of the central nervous system. Sigma 1 receptors may bind psychotropic drugs with high affinity. Sigma 1 receptors exhibit high affinity for (+)-benzomorphans and are typically classified by the receptor ligand specificity. Biol. Cell (2005) 97, 873-883; Current Pharmaceutical Design, 2012, 18, 884-901; Pharmacol. Ther. 2009 November; 124(2): 195-206.

The term “sigma 2 receptor” is used according to its ordinary meaning in the art and refers to a transmembrane protein capable of modulating release of calcium and neurotransmitter systems. A sigma 2 receptor may be expressed in different tissues, and may be concentrated in areas of the central nervous system. Sigma 2 receptors have lower affinity for the (+)-benzomorphans than Sigma 1 receptors and are implicated in apoptosis of cells. The sigma 2 receptor has been implicated in the treatment of AD. WO 2013/029057.

### I. Compositions

Provided herein is a compound having the formula:

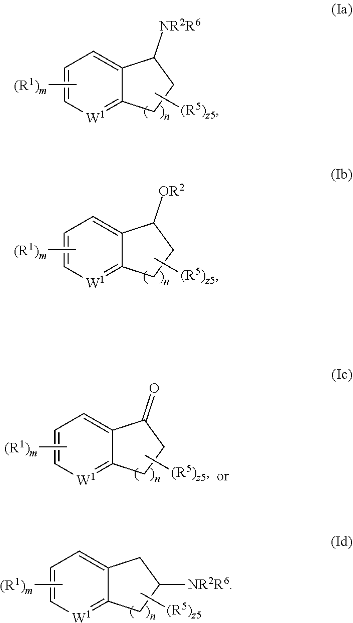

R¹ is halogen (e.g., —F, —Cl, —Br, —I), —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, alkyl, cycloalkyl, alkenyl, cycloalkenyl, alkynyl, heteroalkyl, heterocycloalkyl (e.g., piperazinyl, piperidinyl, morpholinyl), aryl (e.g., phenyl), aralkyl, aralkenyl, heteroaryl (e.g., pyridyl), heteroaralkyl, heteroaralkenyl, or a substituted version of any of these groups. R² is halogen, —N₃, —CF₃, —CCl₃, —CBr₃, —CI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)₂NR⁴R^{4A}, —NHNR⁴R^{4A}, —ONR⁴R^{4A}, —NHC(O)NHNR⁴R^{4A}, substituted or unsubstituted alkyl (e.g., —CH₃, —CH₂CH₃, —CH₂CH₂CH₃, —CH₂CH₂CH₂OH, —CH₂Ph), substituted or unsubstituted heteroalkyl (e.g., —C(O)OCH₂Ph, —C(O)NHCH₂Ph, —CH₂CH₂C(O)OCH₂CH₃, —CH₂CH₂C(O)OCH₃, —CH₂CH₂OCH₂CH₃, —CH₂CH₂OCH₃), substituted or unsubstituted cycloalkyl, alkenyl, cycloalkenyl, or alkynyl, substituted or unsubstituted heterocycloalkyl (e.g., tetrahydropyranyl, piperidinyl, methyl substituted piperidinyl), substituted or unsubstituted aryl, aralkyl, aralkenyl, heteroaryl, heteroaralkyl, or heteroaralkenyl. The symbols n1 and n2 are independently 1 or 2. The symbol m is 1, 2, 3 or 4. n is 1, 2, 3 or 4. R³, R^{3A}, R⁴, R^{4A} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups. R⁵ is halogen, —N₃, —CF₃, —CCl₃, —CBr₃, —CI₃, —CN, —C(O)R^{5C}, —OR^{5D} (e.g., —OH), —NR^{5A}R^{5B}, —C(O)OR^{5D}, —C(O)NR^{5A}R^{5B}, —NO₂, —SR^{5D}, —S(O)ₙ₅R^{5C}, —S(O)ₙ₅OR^{5D}, —S(O)ₙ₅NR^{5A}R^{5B}, —NHNR^{5A}R^{5B}, —ONR^{5A}R^{5B}, —NHC(O)NHNR^{5A}R^{5B} alkyl, cycloalkyl, alkenyl, cycloalkenyl, alkynyl, heteroalkyl, heterocycloalkyl, aryl, aralkyl (e.g. —CH₂Ph), aralkenyl, heteroaryl, heteroaralkyl, heteroaralkenyl, or a substituted version of any of these groups. The symbol n5 is independently 1 or 2. The symbol z5 is independently an integer from 0 to 6. R⁶ is halogen, —N₃, —CF₃, —CCl₃, —CBr₃, —CI₃, —CN, —C(O)R^{6C}, —OR^{6D}, —NR^{6A}R^{6B}, —C(O)OR^{6D}, —C(O)NR^{6A}R^{6B}, —NO₂, —SR^{6D}, —S(O)ₙ₆R^{6C}, —S(O)ₙ₆OR^{6D}, —S(O)ₙ₆NR^{6A}R^{6B}, —NHNR^{6A}R^{6B}, —ONR^{6A}R^{6B}, —NHC(O)NHNR^{6A}R^{6B}, substituted or unsubstituted alkyl (e.g., —CH₃, —CH₂CH₃, —CH₂CH₂CH₃, —CH₂CH₂CH₂OH, —CH₂Ph), substituted or unsubstituted heteroalkyl (e.g., —C(O)OCH₂Ph, —CH₂CH₂C(O)OCH₂CH₃, —CH₂CH₂C(O)OCH₃, —CH₂CH₂OCH₂CH₃, —CH₂CH₂OCH₃), substituted or unsubstituted cycloalkyl, alkenyl, cycloalkenyl, or alkynyl, substituted or unsubstituted heterocycloalkyl (e.g., tetrahydropyranyl, piperidinyl, methyl substituted piperidinyl), substituted or unsubstituted aryl, aralkyl, aralkenyl, heteroaryl, heteroaralkyl, or heteroaralkenyl. The symbol n6 is independently 1 or 2. W¹ is CH, C(R¹), or N; and R^{5A}, R^{5B}, R^{5C}, R^{5D}, R^{6A}, R^{6B}R^{6C}, and R^{6D} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

In embodiments the compound has the formula:

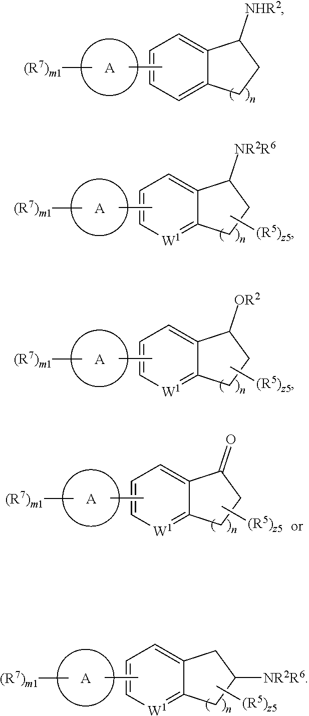

In embodiments, ring A is —X₁—Y₁—Z₁—; and

m1 is 0, 1, 2, 3, or 4. In embodiments, Ring A is heterocycloalkylene (e.g., piperazinyl, piperidinyl, morpholinyl). In embodiments, Ring A is arylene (e.g., phenyl).

R⁷ is —CF₃, —CN, —OH, —NH₂, —CONH₂, —S(O)₃H, —S(O)₂NH₂, —NHC(O) NH₂, —NHC(O)H, —OCHF₂, substituted or unsubstituted alkyl (e.g., —CH₃, —CH₂CH₃, —CH₂CH₂CH₃, CH₂CH₂CH₂OH, —CH₂C(CH₃)₂), substituted or unsubstituted heteroalkyl (e.g., —CH₂CH₂C(O)OCH₂CH₃, —CH₂C(O)OCH₂CH₃, —CH₂CH₂C(O)OCH₃, —CH₂CH₂OCH₂CH₃, —CH₂CH₂OCH₃), substituted or unsubstituted cycloalkyl (e.g., unsubstituted cyclopentyl, unsubstituted cyclohexyl, unsubstituted cyclobutyl), substituted or unsubstituted heterocycloalkyl, substituted or unsubstituted aryl, or substituted or unsubstituted heteroaryl. Ring A is arylene, heteroarylene, cycloalkylene, or heterocycloalkylene. The symbol m1 is 0, 1, 2, 3, or 4.

In embodiments, R⁷ is

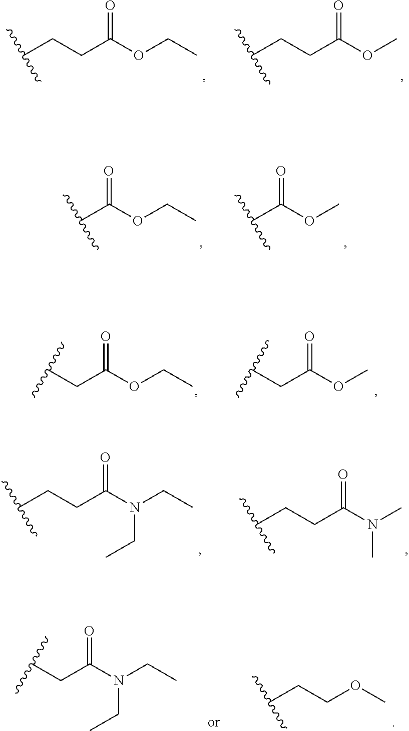

In embodiments, R⁷ is

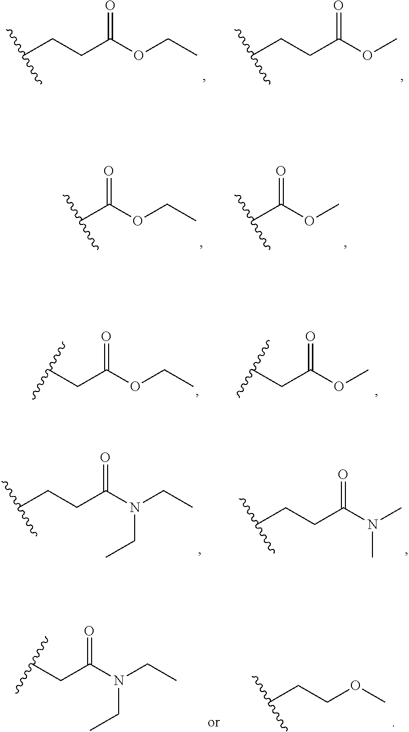

R¹ may be —Cl, —F, —Br, substituted or unsubstituted C₁-C₁₀ alkyl, substituted or unsubstituted 3 to 6 membered heterocycloalkyl (e.g., piperazinyl, piperidinyl, morpholinyl). In embodiments, R³ is oxo, substituted or unsubstituted alkyl (e.g., methyl, ethyl, n-propyl, —CH₂CH₂OH, —C(O)CH₃, —CH₃, —CH₂CH₃, —CH₂CH₂CH₃, CH₂CH₂CH₂OH, —CH₂C(CH₃)₂), or substituted or unsubstituted heteroalkyl (e.g., —CH₂CH₂C(O)OCH₂CH₃, —CH₂CH₂OCH₂CH₃, —CH₂CH₂OCH₃, —CH₂C(O)OCH₂CH₃, —CH₂CH₂C(O)OCH₃). In embodiments, R⁷ is oxo, —OH, unsubstituted alkyl, unsubstituted heteroalkyl (e.g., —OCH₂CH₃, —OCH₃), or unsubstituted cycloalkyl (e.g., cyclopentyl, cyclohexyl, cyclobutyl).

In embodiments, R¹ is

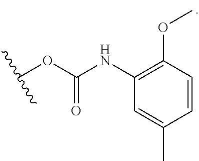 [SMILES: *OC(=O)Nc1cc(C)ccc1OC]

In embodiments, R¹ is

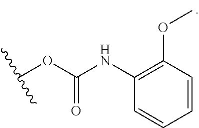 [SMILES: *OC(=O)Nc1ccccc1OC]

In embodiments, R¹ is

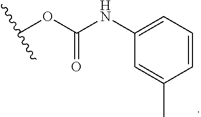 [SMILES: *OC(=O)Nc1cccc(C)c1]

In embodiments, R¹ is

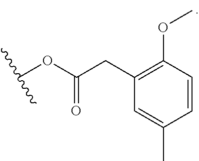 [SMILES: *OC(=O)Cc1cc(C)ccc1OC]

In embodiments, R¹ is

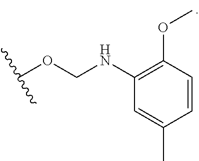 [SMILES: *OCNc1cc(C)ccc1OC]

R¹ of the compounds described herein may be Cl, F, Br, —OH, —OR³, —NR³R^{3A}, substituted or unsubstituted C₁-C₁₀ alkyl, substituted or unsubstituted 3 to 6 membered heterocycloalkyl (e.g., piperazinyl, piperidinyl, morpholinyl), substituted or unsubstituted aryl (e.g., phenyl), substituted or unsubstituted heteroaryl. In embodiments, R³ is oxo, halogen, —CF₃, —CN, —OH, —NH₂, —COOH, —CONH₂, —NO₂, —SH, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² may be substituted or unsubstituted alkyl. R² may be substituted alkyl. R² may be substituted or unsubstituted C₁-C₂₀ alkyl. R² may be substituted C₁-C₂₀ alkyl. R² may be substituted or unsubstituted C₁-C₁₀ alkyl. R² may be substituted C₁-C₁₀ alkyl. R² may be substituted or unsubstituted C₁-C₅ alkyl. R² may be substituted C₁-C₅ alkyl. R² may be substituted or unsubstituted methyl, substituted or unsubstituted ethyl, or substituted or unsubstituted propyl.

R² may be substituted or unsubstituted aralkyl. R² may be substituted aralkyl. R² may be substituted or unsubstituted C₁-C₂₀ aralkyl. R² may be substituted C₁-C₂₀ aralkyl. R² may be substituted or unsubstituted C₁-C₁₀ aralkyl. R² may be substituted C₁-C₁₀ aralkyl. R² may be —C(O)OCH₂Ph.

In embodiments, R² is

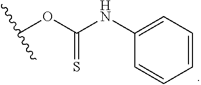 [SMILES: *OC(=S)Nc1ccccc1]

In embodiments, R² is

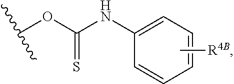

R wherein R^{4B} is —CF₃, —CN, —OH, unsubstituted alkyl or unsubstituted heteroalkyl. In embodiments, R² is

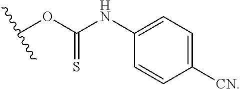 [SMILES: *OC(=S)Nc1ccc(C#N)cc1]

In embodiments, R² is

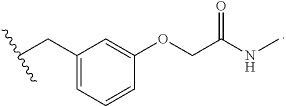 [SMILES: *Cc1cccc(OCC(=O)NC)c1]

In embodiments, R² is

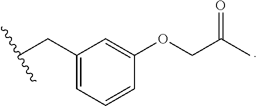 [SMILES: *Cc1cccc(OCC(C)=O)c1]

In embodiments, R² is

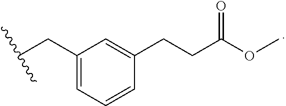 [SMILES: *Cc1cccc(CCC(=O)OC)c1]

In embodiments, R² is

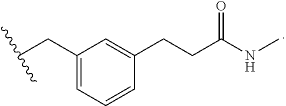 [SMILES: *Cc1cccc(CCC(=O)NC)c1]

In embodiments, R² is a substituted C₁-C₆ alkyl or a substituted 2 to 6 membered heteroalkyl, wherein the substitution is a silyl ether (e.g., trimethylsilyl ether (TMS), triethylsilyl ether (TES), tert-butyldimethylsilyl ether (TBS), tert-butyldiphenylsilyl ether (TBDPS), or triisopropylsilyl ether (TIPS)).

R² may be —C(O)OR⁴.

R² may be substituted or unsubstituted alkyl (e.g., methyl). R² may be substituted or unsubstituted heterocycloalkyl (e.g., tetrahydropyranyl, piperidinyl, methyl substituted piperidinyl).

R⁴ may independently be —CF₃, —CN, —OH, —NH₂, —COOH, —CONH₂, —S(O)₃H, —S(O)₂NH₂, —NHC(O) NH₂, —NHC(O)H, substituted or unsubstituted aralkyl (—CH₂Ph), substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Provided herein are compositions having the formula:

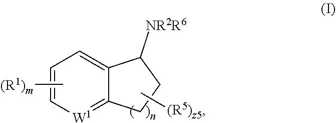

R¹ is hydrogen, halogen, —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups. R² is hydrogen, halogen, —N₃, —CF₃, —CCl₃, —CBr₃, —CI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙ₂NR⁴R^{4A}, —NHNR⁴R^{4A}, —ONR⁴R^{4A}, —NHC(O)NHNR⁴R^{4A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups. The symbols n1 and n2 are independently 1 or 2. The symbol m is 1, 2, 3 or 4. They symbol n is 1 or 2. R³, R^{3A}, R⁴, R^{4A} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups. R⁶ is halogen, —N₃, —CF₃, —CCl₃, —CBr₃, —CI₃, —CN, —C(O)R^{6C}, —OR^{6D}, —NR^{6A}R^{6B}, —C(O)OR^{6D}, —C(O)NR^{6A}R^{6B}, —NO₂, —SR^{6D}, —S(O)ₙ₆R^{6C}, —S(O)ₙ₆OR^{6D}, —S(O)ₙ₆NR^{6A}R^{6B}, —NHNR^{6A}R^{6B}, —ONR^{6A}R^{6B}, —NHC(O)NHNR^{6A}R^{6B}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

The compound of formula (I) may have formula:

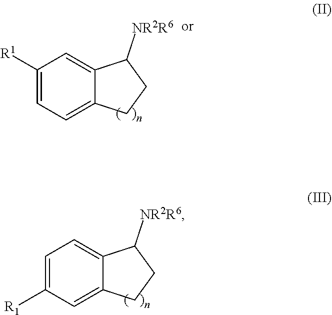

where R¹, R², and R⁶ are as described herein.

In embodiments, R¹ is halogen, —OR³, —NR³R^{3A}, —C(O)OR³, alkyl, cycloalkyl, heterocycloalkyl, aryl, aralkyl, aralkenyl, heteroaryl, heteroaralkyl, heteroaralkenyl, or a substituted version of any of these groups. In embodiments, R¹ is halogen, —OR³, —NR³R^{3A}, alkyl, cycloalkyl, heterocycloalkyl, aryl, aralkyl, aralkenyl, heteroaryl, heteroaralkyl, heteroaralkenyl, or a substituted version of any of these groups. In embodiments, R¹ is halogen.

In embodiments, R¹ is Cl, F, Br, —OH, —OR³, —NR³R^{3A}, substituted or unsubstituted C₁-C₁₀ alkyl, substituted or unsubstituted heterocycloalkyl, substituted or unsubstituted aryl, substituted or unsubstituted heteroaryl; R^{3A} is hydrogen; R³ is —CF₃, —CN, —OH, —NH₂, —CONH₂, —S(O)₃H, —S(O)₂NH₂, —NHC(O) NH₂, —NHC(O)H, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups. In embodiments, R³ is oxo.

In embodiments, R¹ is

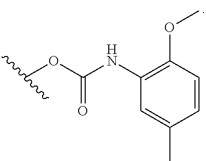 [SMILES: *OC(=O)Nc1cc(C)ccc1OC]

In embodiments, R¹ is

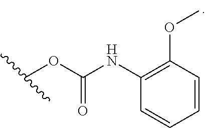 [SMILES: *OC(=O)Nc1ccccc1OC]

In embodiments, R¹ is

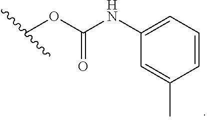 [SMILES: *OC(=O)Nc1cccc(C)c1]

In embodiments, R¹ is

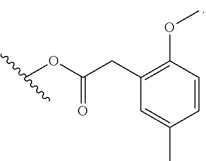 [SMILES: *OC(=O)Cc1cc(C)ccc1OC]

In embodiments, R¹ is

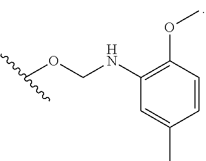 [SMILES: *OCNc1cc(C)ccc1OC]

In embodiments, R² is halogen, —CN, —C(O)R⁴, —OR⁴, —NR⁴R^{4A}, —C(O)OR⁴, —C(O)NR⁴R^{4A}, —S(O)ₙ₂R⁴, —S(O)ₙ₂OR⁴, —S(O)ₙ₂NR⁴R^{4A}, —ONR⁴R^{4A}, —NHC(O)NHNR⁴R^{4A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

In embodiments, R² is

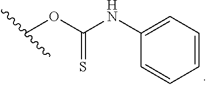 [SMILES: *OC(=S)Nc1ccccc1]

In embodiments, R² is

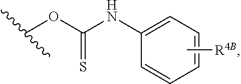

wherein R^{4B} is —CF₃, —CN, —OH, unsubstituted alkyl or unsubstituted heteroalkyl. In embodiments, R² is

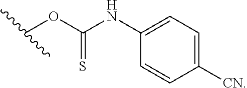 [SMILES: *OC(=S)Nc1ccc(C#N)cc1]

In embodiments, R² is

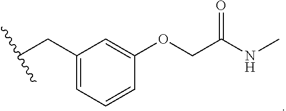 [SMILES: *Cc1cccc(OCC(=O)NC)c1 ; "."]

In embodiments, R² is

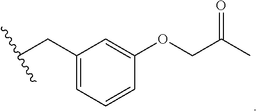 [SMILES: *Cc1cccc(OCC(C)=O)c1 ; "."]

In embodiments, R² is

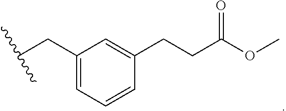 [SMILES: *Cc1cccc(CCC(=O)OC)c1 ; "."]

In embodiments, R² is

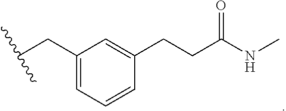 [SMILES: *Cc1cccc(CCC(=O)NC)c1 ; "."]

In embodiments, R² is a substituted C₁-C₆ alkyl, wherein the substitution is a silyl ether (e.g., trimethylsilyl ether (TMS), triethylsilyl ether (TES), tert-butyldimethylsilyl ether (TBS), tert-butyldiphenylsilyl ether (TBDPS), or triisopropylsilyl ether (TIPS)).

The compound of formula (I) may have the formula:

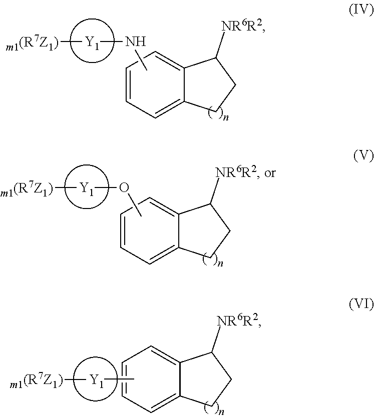

where R², R⁷, ring A, n, and m1 are as described herein. In embodiments, R⁷ is —CF₃, —CN, —OH, —NH₂, —CONH₂, —S(O)₃H, —S(O)₂NH₂, —NHC(O)NH₂, —NHC(O)H, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups; ring A is arylene, heteroarylene, cycloalkylene or heterocycloalkylene; and m1 is 0, 1, 2, 3, or 4. In embodiments, ring A is arylene or heterocycloalkylene.

The compound of formula (I) may have the formula:

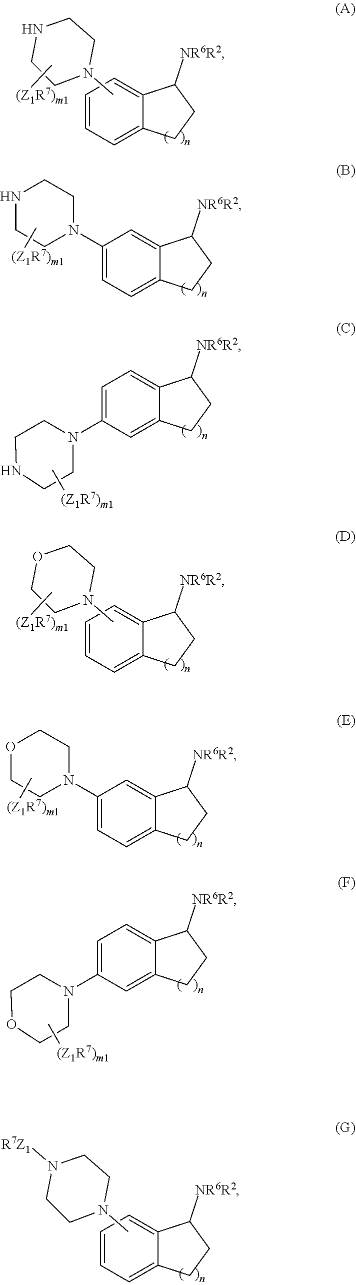

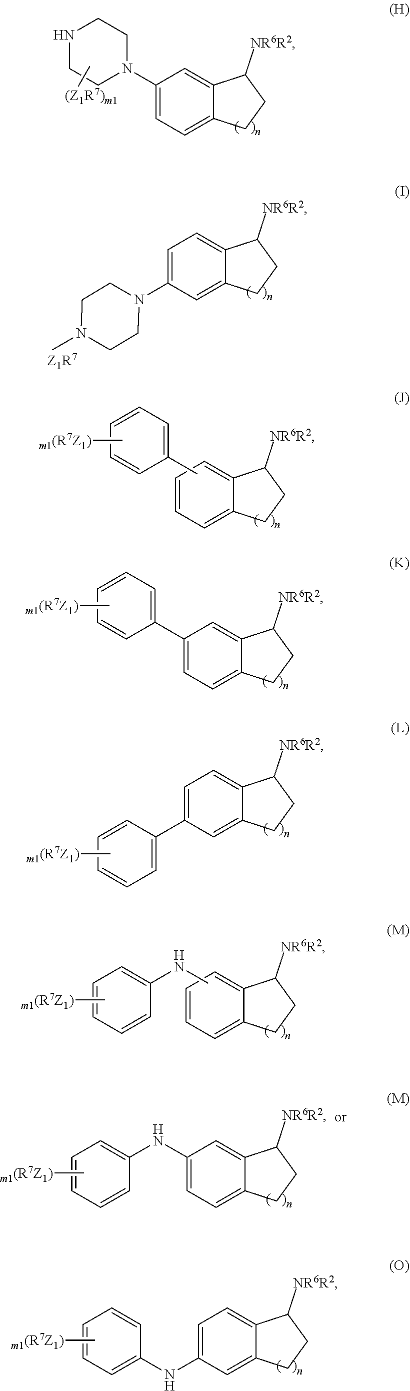

where R², R⁶, R⁷, n, and m1 are as described herein.

The compound of formula (I) may have the formula:

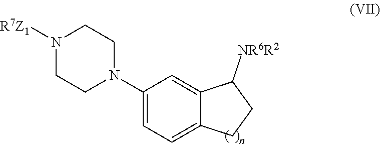

where R², R⁶, and R⁷ are as described herein. R⁷ of formula (VII) may be substituted or unsubstituted alkyl. R⁷ of formula (VII) may be substituted or unsubstituted C₁-C₁₀ alkyl. R⁷ of formula (VII) may be unsubstituted C₁-C₁₀ alkyl. R⁷ of formula (VII) may be substituted C₁-C₁₀ alkyl. R⁷ of formula (VII) may be substituted C₁-C₁₀ alkyl. R⁷ of formula (VII) may be methyl. R² of formula (VII) may be —C(O)OR⁴, where R⁴ is substituted or unsubstituted aralkyl. R² of formula (VII) may be —C(O)OR⁴, where R⁴ is substituted or unsubstituted aralkyl. R⁴ of formula (VII) may be unsubstituted benzyl.

In embodiments, R⁷ is halogen, —CF₃, —CN, —OH, unsubstituted alkyl or unsubstituted heteroalkyl. In embodiments, R⁷ is halogen, —CF₃, —OH, —OCH₃ or unsubstituted C₁-C₅ alkyl. In embodiments, m1 is 0 or 1. In embodiments, m1 is 0. In embodiments, m1 is 1. In embodiments, n is 1.

In embodiments, R² is —OR⁴, —NR⁴R^{4A}, —C(O)OR⁴, alkyl, cycloalkyl, alkenyl, cycloalkenyl, alkynyl, heterocycloalkyl, aryl, aralkyl, aralkenyl, heteroaryl, heteroaralkyl, heteroaralkenyl, or a substituted version of any of these groups. In embodiments, R² is —OR⁴, —NR⁴R^{4A}, —C(O)OR⁴, alkyl, heterocycloalkyl, aryl, aralkyl, aralkenyl, heteroaryl, heteroaralkyl, heteroaralkenyl, or a substituted version of any of these groups. In embodiments, R² is —C(O)OR⁴, wherein R⁴ is substituted or unsubstituted aralkyl. In embodiments, R² is unsubstituted C₁-C₅ alkyl. In embodiments, R⁴ is unsubstituted aralkyl.

Ring A may be arylene, heteroarylene, cycloalkylene, or heterocycloalkylene. Ring A may be arylene or heterocycloalkylene. Ring A may be arylene. Ring A may be 5 to 7 membered arylene. Ring A may be 5 or 6 membered arylene. Ring A may be 5 membered arylene. Ring A may be 6 membered arylene. Ring A may be heterocycloalkylene. Ring A may be 3 to 10 membered heterocycloalkylene. Ring A may be 3 to 8 membered heterocycloalkylene. Ring A may be 3 to 6 membered heterocycloalkylene. Ring A may be 3 membered heterocycloalkylene. Ring A may be 4 membered heterocycloalkylene. Ring A may be 5 membered heterocycloalkylene. Ring A may be 6 membered heterocycloalkylene.

The symbol n may be 1. The symbol n may be 2. The symbol n1 may be 1. The symbol n1 may be 2. The symbol n2 may be 1. The symbol n2 may be 2. The symbol m may be 1. The symbol m may be 2. The symbol m may be 3. The symbol m may be 4. The symbol m1 may be 0 or 1. The symbol m1 may be 0. The symbol m1 may be 1. The symbol m1 may be 2. The symbol m1 may be 3. The symbol m1 may be 4.

R¹ may be substituted or unsubstituted alkyl. R¹ may be substituted alkyl. R¹ may be unsubstituted alkyl. R¹ may be substituted or unsubstituted C₁-C₂₀ alkyl. R¹ may be substituted C₁-C₂₀ alkyl. R¹ may be unsubstituted C₁-C₂₀ alkyl. R¹ may be substituted or unsubstituted C₁-C₁₀ alkyl. R¹ may be substituted C₁-C₁₀ alkyl. R¹ may be unsubstituted C₁-C₁₀ alkyl. R¹ may be substituted or unsubstituted C₁-C₅ alkyl. R¹ may be substituted C₁-C₅ alkyl. R¹ may be unsubstituted C₁-C₅ alkyl. R¹ may be methyl, substituted or unsubstituted ethyl, or substituted or unsubstituted propyl. R¹ may be hydrogen. R¹ may be methyl. R¹ may be methyl, substituted or unsubstituted ethyl, or substituted or unsubstituted propyl.

R¹ may be substituted or unsubstituted heteroalkyl. R¹ may be substituted heteroalkyl. R¹ may be unsubstituted heteroalkyl. R¹ may be substituted or unsubstituted 2 to 20 membered heteroalkyl. R¹ may be substituted 2 to 20 membered heteroalkyl. R¹ may be substituted or unsubstituted 2 to 10 membered heteroalkyl. R¹ may be substituted 2 to 10 membered heteroalkyl. R¹ may be substituted or unsubstituted 2 to 6 membered heteroalkyl. R¹ may be substituted 2 to 6 membered heteroalkyl.

R¹ may be substituted or unsubstituted cycloalkyl. R¹ may be substituted cycloalkyl. R¹ may be unsubstituted cycloalkyl. R¹ may be substituted or unsubstituted 3 to 20 membered cycloalkyl. R¹ may be substituted 3 to 20 membered cycloalkyl. R¹ may be substituted or unsubstituted 3 to 10 membered cycloalkyl. R¹ may be substituted 3 to 10 membered cycloalkyl. R¹ may be substituted or unsubstituted 3 to 6 membered cycloalkyl. R¹ may be substituted 3 to 6 membered cycloalkyl.

R¹ may be substituted or unsubstituted heterocycloalkyl. R¹ may be substituted heterocycloalkyl. R¹ may be unsubstituted heterocycloalkyl. R¹ may be substituted or unsubstituted 3 to 20 membered heterocycloalkyl. R¹ may be substituted 3 to 20 membered heterocycloalkyl. R¹ may be substituted or unsubstituted 3 to 10 membered heterocycloalkyl. R¹ may be substituted 3 to 10 membered heterocycloalkyl. R¹ may be substituted or unsubstituted 3 to 6 membered heterocycloalkyl. R¹ may be substituted 3 to 6 membered heterocycloalkyl.

R¹ may be substituted or unsubstituted aryl. R¹ may be substituted aryl. R¹ may be unsubstituted aryl. R¹ may be substituted or unsubstituted 5 to 20 membered aryl. R¹ may be substituted 5 to 20 membered aryl. R¹ may be substituted or unsubstituted 5 to 8 membered aryl. R¹ may be substituted 5 to 8 membered aryl. R¹ may be substituted or unsubstituted 5 or 6 membered aryl. R¹ may be substituted 5 or 6 membered aryl (e.g. phenyl).

R¹ may be substituted or unsubstituted heteroaryl. R¹ may be substituted heteroaryl. R¹ may be unsubstituted heteroaryl. R¹ may be substituted or unsubstituted 5 to 20 membered heteroaryl. R¹ may be substituted 5 to 20 membered heteroaryl. R¹ may be substituted or unsubstituted 5 to 8 membered heteroaryl. R¹ may be substituted 5 to 8 membered heteroaryl. R¹ may be substituted or unsubstituted 5 or 6 membered heteroaryl. R¹ may be substituted 5 or 6 membered heteroaryl.

R¹ may be halogen, —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups. When the compound is a compound having formula (II) or formula (III), R¹ may be halogen, —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

R¹ may be halogen, —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups. R¹ may be halogen. When the compound is a compound having formula (II) or formula (III), R¹ may be halogen, —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)ₙ₁R³, —S(O)ₙ₁OR³, —S(O)ₙ₁NR³R^{3A}, —NHNR³R^{3A}, —ONR³R^{3A}, —NHC(O)NHNR³R^{3A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

R¹ may be halogen, —OR³, —NR³R^{3A}, —C(O)OR³, alkyl, cycloalkyl, heterocycloalkyl, aryl, heteroaryl, or a substituted version of any of these groups.

R¹ may be halogen, —OR³, —NR³R^{3A}, alkyl, heterocycloalkyl, aryl, heteroaryl, or a substituted version of any of these groups.

R¹ of the compounds described herein (e.g. formula (I), (II), (III)) may be Cl, F, Br, —OH, —OR³, —NR³R^{3A}, substituted or unsubstituted C₁-C₁₀ alkyl, substituted or unsubstituted heterocycloalkyl, substituted or unsubstituted aryl, substituted or unsubstituted heteroaryl, were R^{3A} is hydrogen, and R³ is oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

R¹ of the compounds described herein (e.g. formula (I), (II), or (III)) may be Cl, F, Br, —OH, —OR³, —NR³R^{3A}, substituted or unsubstituted C₁-C₁₀ alkyl, substituted or unsubstituted heterocycloalkyl, substituted or unsubstituted aryl, substituted or unsubstituted heteroaryl, were R^{3A} is hydrogen, R³ is —CF₃, —CN, —OH, —NH₂, —CONH₂, —S(O)₃H, —S(O)₂NH₂, —NHC(O) NH₂, —NHC(O)H, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

R³ is independently oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

R³ may independently be —CF₃, —CN, —OH, —NH₂, —COOH, —CONH₂, —S(O)₃H, —S(O)₂NH₂, —NHC(O) NH₂, —NHC(O)H, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

R² may be substituted or unsubstituted alkyl. R² may be substituted alkyl. R² may be unsubstituted alkyl. R² may be substituted or unsubstituted C₁-C₂₀ alkyl. R² may be substituted C₁-C₂₀ alkyl. R² may be unsubstituted C₁-C₂₀ alkyl. R² may be substituted or unsubstituted C₁-C₁₀ alkyl. R² may be substituted C₁-C₁₀ alkyl. R² may be unsubstituted C₁-C₁₀ alkyl. R² may be substituted or unsubstituted C₁-C₅ alkyl. R² may be substituted C₁-C₅ alkyl. R² may be unsubstituted C₁-C₅ alkyl. R² may be methyl, substituted or unsubstituted ethyl, or substituted or unsubstituted propyl. R² may be hydrogen. R² may be methyl.

R² may be substituted or unsubstituted heteroalkyl. R² may be substituted heteroalkyl. R² may be unsubstituted heteroalkyl. R² may be substituted or unsubstituted 2 to 20 membered heteroalkyl. R² may be substituted 2 to 20 membered heteroalkyl. R² may be substituted or unsubstituted 2 to 10 membered heteroalkyl. R² may be substituted 2 to 10 membered heteroalkyl. R² may be substituted or unsubstituted 2 to 6 membered heteroalkyl. R² may be substituted 2 to 6 membered heteroalkyl.

R² may be substituted or unsubstituted cycloalkyl. R² may be substituted cycloalkyl. R² may be unsubstituted cycloalkyl. R² may be substituted or unsubstituted 3 to 20 membered cycloalkyl. R² may be substituted 3 to 20 membered cycloalkyl. R² may be substituted or unsubstituted 3 to 10 membered cycloalkyl. R² may be substituted 3 to 10 membered cycloalkyl. R² may be substituted or unsubstituted 3 to 6 membered cycloalkyl. R² may be substituted 3 to 6 membered cycloalkyl.

R² may be substituted or unsubstituted heterocycloalkyl. R² may be substituted heterocycloalkyl. R² may be unsubstituted heterocycloalkyl. R² may be substituted or unsubstituted 3 to 20 membered heterocycloalkyl. R² may be substituted 3 to 20 membered heterocycloalkyl. R² may be substituted or unsubstituted 3 to 10 membered heterocycloalkyl. R² may be substituted 3 to 10 membered heterocycloalkyl. R² may be substituted or unsubstituted 3 to 6 membered heterocycloalkyl. R² may be substituted 3 to 6 membered heterocycloalkyl.

R² may be substituted or unsubstituted aryl. R² may be substituted aryl. R² may be unsubstituted aryl. R² may be substituted or unsubstituted 5 to 20 membered aryl. R² may be substituted 5 to 20 membered aryl. R² may be substituted or unsubstituted 5 to 8 membered aryl. R² may be substituted 5 to 8 membered aryl. R² may be substituted or unsubstituted 5 or 6 membered aryl. R² may be substituted 5 or 6 membered aryl (e.g. phenyl).

R² may be substituted or unsubstituted heteroaryl. R² may be substituted heteroaryl. R² may be unsubstituted heteroaryl. R² may be substituted or unsubstituted 5 to 20 membered heteroaryl. R² may be substituted 5 to 20 membered heteroaryl. R² may be substituted or unsubstituted 5 to 8 membered heteroaryl. R² may be substituted 5 to 8 membered heteroaryl. R² may be substituted or unsubstituted 5 or 6 membered heteroaryl. R² may be substituted 5 or 6 membered heteroaryl.

R² may be halogen, —N₃, —CF₃, —CCl₃, —CBr₃, —CI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙ₂NR⁴R^{4A}, —NHNR⁴R^{4A}, —ONR⁴R^{4A}, —NHC(O)NHNR⁴R^{4A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups. R² of the compound of formula (II) or formula (III) may be halogen, —N₃, —CF₃, —CCl₃, —CBr₃, —CI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙ₂NR⁴R^{4A}, —NHNR⁴R^{4A}, —ONR⁴R^{4A}, —NHC(O)NHNR⁴R^{4A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

R² may be halogen, —CN, —C(O)R⁴, —OR⁴, —NR⁴R^{4A}, —C(O)OR⁴, —C(O)NR⁴R^{4A}, —S(O)ₙ₂R⁴, —S(O)ₙ₂OR⁴, —S(O)ₙ₂NR⁴R^{4A}, —ONR⁴R^{4A}, —NHC(O)NHNR⁴R^{4A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

R² may be halogen, —OR⁴, —NR⁴R^{4A}, —C(O)OR⁴, alkyl, cycloalkyl, alkenyl, cycloalkenyl, alkynyl, heterocycloalkyl, aryl, aralkyl, aralkenyl, heteroaryl, heteroaralkyl, heteroaralkenyl, or a substituted version of any of these groups. R² may be —OR⁴, —NR⁴R^{4A}, —C(O)OR⁴, alkyl, cycloalkyl, alkenyl, cycloalkenyl, alkynyl, heterocycloalkyl, aryl, aralkyl, aralkenyl, heteroaryl, heteroaralkyl, heteroaralkenyl, or a substituted version of any of these groups.

R² may be halogen, —OR⁴, —NR⁴R^{4A}, —C(O)OR⁴, alkyl, heterocycloalkyl, aryl, aralkyl, heteroaryl, or a substituted version thereof. R² may be —OR⁴, —NR⁴R^{4A}, —C(O)OR⁴, alkyl, heterocycloalkyl, aryl, aralkyl, heteroaryl, or a substituted version thereof.

R² may be —C(O)OR⁴, where R⁴ is as described herein. R² may be —C(O)OR⁴, where R⁴ is aryl, aralkyl, or a substituted version of either group. R² may be —C(O)OR⁴, where R⁴ is substituted or unsubstituted aryl or aralkyl. R⁴ may be unsubstituted aralkyl. R⁴ may be substituted or unsubstituted benzyl. R⁴ may be unsubstituted benzyl. R² may be substituted or unsubstituted aralkyl.

R⁴ may independently be —CF₃, —CN, —OH, —NH₂, —COOH, —CONH₂, —S(O)₃H, —S(O)₂NH₂, —NHC(O) NH₂, —NHC(O)H, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

The compound may have the formula as set forth in Table A:

TABLE A

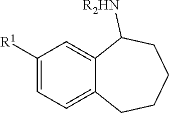

| R¹ | R² | R¹ | R² |
|---|---|---|---|
| 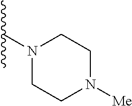 [SMILES: *N1CCN(C)CC1] | Cbz | 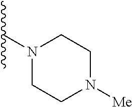 [SMILES: *N1CCN(C)CC1] | Alloc |
| C₈H₂₀N₂ | | C₈H₂₀N₂ | |
| 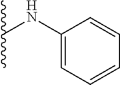 [SMILES: *Nc1ccccc1] | Bn | 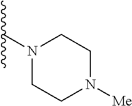 [SMILES: *N1CCN(C)CC1] | 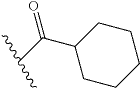 [SMILES: *C(=O)C1CCCCC1] |
| C₉H₁₅N | | C₈H₂₀N₂ | |
| 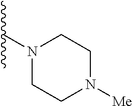 [SMILES: *N1CCN(C)CC1] | H | 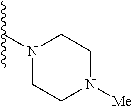 [SMILES: *N1CCN(C)CC1] | 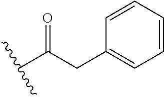 [SMILES: *C(=O)Cc1ccccc1] |
| C₈H₂₀N₂ | | C₈H₂₀N₂ | |
| 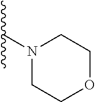 [SMILES: *N1CCOCC1] | 4-dimethylaminobenzyl | 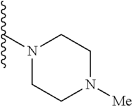 [SMILES: *N1CCN(C)CC1] | 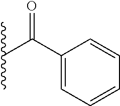 [SMILES: *C(=O)c1ccccc1] |
| C₇H₁₇NC | | C₈H₂₀N₂ | |
| 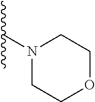 [SMILES: *N1CCOCC1] | 3,4-dimethyloxybenzyl | 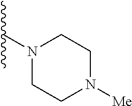 [SMILES: *N1CCN(C)CC1] | 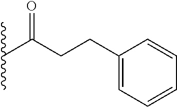 [SMILES: *C(=O)CCc1ccccc1] |
| C₇H₁₇NC | | C₈H₂₀N₂ | |
| 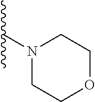 [SMILES: *N1CCOCC1] | 3,4-dichlorobenzyl | 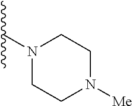 [SMILES: *N1CCN(C)CC1] | 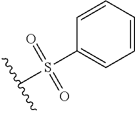 [SMILES: *S(=O)(=O)c1ccccc1] |
| C₇H₁₇NC | | C₈H₂₀N₂ | |
| 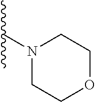 [SMILES: *N1CCOCC1] | 2-fluorobenzyl | 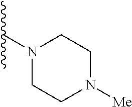 [SMILES: *N1CCN(C)CC1] | 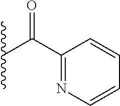 [SMILES: *C(=O)c1ccccn1] |
| C₇H₁₇NC | | C₈H₂₀N₂ | |
| 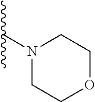 [SMILES: *N1CCOCC1] | methyl | 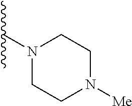 [SMILES: *N1CCN(C)CC1] | 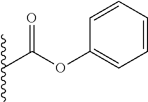 [SMILES: *C(=O)Oc1ccccc1] |
| C₇H₁₇NC | | C₈H₂₀N₂ | |
| 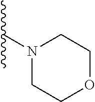 [SMILES: *N1CCOCC1] | Phenethyl | 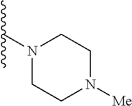 [SMILES: *N1CCN(C)CC1] | 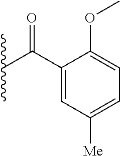 [SMILES: *C(=O)c1cc(C)ccc1OC] |
| C₇H₁₇NC | | C₈H₂₀N₂ | |
| 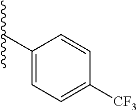 [SMILES: *c1ccc(C(F)(F)F)cc1] | Bn | 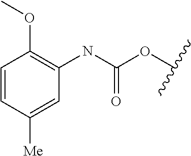 [SMILES: *OC(=O)Nc1cc(C)ccc1OC] | Bn |
| C₁₀H₁₃F₃ | | | |
| 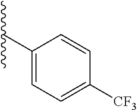 [SMILES: *c1ccc(C(F)(F)F)cc1] | H | OH | Bn |
| C₁₀H₁₃F₃ | | | |
| 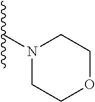 [SMILES: *N1CCOCC1] | 3,5-dichlorobenzyl | OH | Cbz |
| C₇H₁₇NO | | | |
| 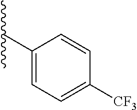 [SMILES: *c1ccc(C(F)(F)F)cc1] | Methyl | 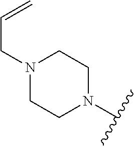 [SMILES: *N1CCN(CC=C)CC1] | Cbz |
| C₁₀H₁₃F₃ | | | |
| 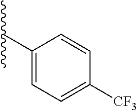 [SMILES: *c1ccc(C(F)(F)F)cc1] | 4-dimethylaminobenzyl | BuNH | Bn |
| C₁₀H₁₃F₃ | | | |
| 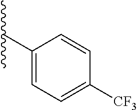 [SMILES: *c1ccc(C(F)(F)F)cc1] | 3,4-dimethoxybenzyl | H | CO2Me |
| C₁₀H₁₃F₃ | | | |
| 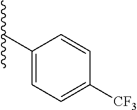 [SMILES: *c1ccc(C(F)(F)F)cc1] | 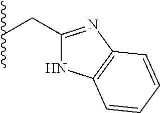 [SMILES: *Cc1nc2ccccc2[nH]1] | H | H |
| C₁₀H₁₃F₃ | C₁₁H₁₆N₂ | | |
| 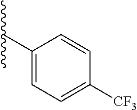 [SMILES: *c1ccc(C(F)(F)F)cc1] | 3,4-dichlorobenzyl | H | Bn |
| C₁₀H₁₃F₃ | | | |
| 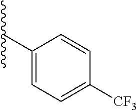 [SMILES: *c1ccc(C(F)(F)F)cc1] | phenethyl | H | 4-chlorobenzyl |
| C₁₀H₁₃F₃ | | | |
| 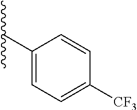 [SMILES: *c1ccc(C(F)(F)F)cc1] | 2-fluorobenzyl | H | 3,4-methylenedioxyphenyl |
| C₁₀H₁₃F₃ | | | |
| 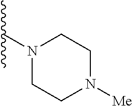 [SMILES: *N1CCN(C)CC1] | 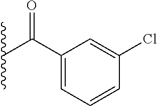 [SMILES: *C(=O)c1cccc(Cl)c1] | H | Me |
| C₈H₂₀N₂ | C₁₀H₁₃ClO | | |
| 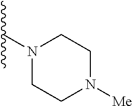 [SMILES: *N1CCN(C)CC1] | 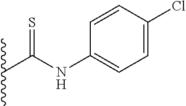 [SMILES: *C(=S)Nc1ccc(Cl)cc1] | H | Phenethyl |
| C₈H₂₀N₂ | C₁₁H₁₄N₂S | | |
| 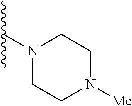 [SMILES: *N1CCN(C)CC1] | COMe | 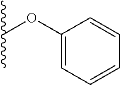 [SMILES: *Oc1ccccc1] | Bn |
| C₈H₂₀N₂ | | C₉H₁₄O | |
| 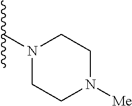 [SMILES: *N1CCN(C)CC1] | 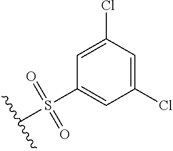 [SMILES: *S(=O)(=O)c1cc(Cl)cc(Cl)c1] | 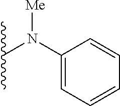 [SMILES: *N(C)c1ccccc1] | Cbz |
| C₈H₂₀N₂ | | C₁₀H₁₇N | |
| OH | Bn | 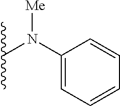 [SMILES: *N(C)c1ccccc1] | Bn |
| | | C₁₀H₁₇N | |

The compound may have the formula as set forth in Table B:

TABLE B

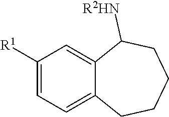

| R1 | R2 |
|---|---|
| 3-methoxyphenyl | Cbz |
| 3-methoxyphenyl | Bn |
| 3-hydroxyphenyl | Cbz |
| 3-hydroxyphenyl | Bn |
| 4-methoxyphenyl | Cbz |
| 4-methoxyphenyl | Bn |

The compound may have the formula as set forth in Table C:

TABLE C

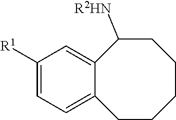

| R¹ | R² | R¹ | R² |
|---|---|---|---|
| Cl | 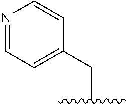 [SMILES: *Cc1ccncc1] | 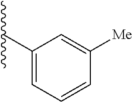 [SMILES: *c1cccc(C)c1] | 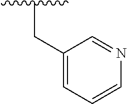 [SMILES: *Cc1cccnc1] |
| Cl | 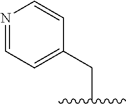 [SMILES: *Cc1ccncc1] | 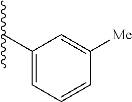 [SMILES: *c1cccc(C)c1] | 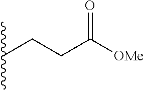 [SMILES: *CCC(=O)OC] |
| Cl | 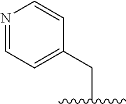 [SMILES: *Cc1ccncc1] | 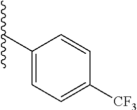 [SMILES: *c1ccc(C(F)(F)F)cc1] | Cbz |
| Cl | 4-dimethylaminobenzyl | 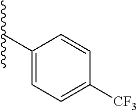 [SMILES: *c1ccc(C(F)(F)F)cc1] | H |
| Cl | 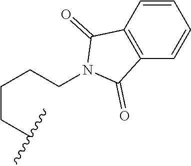 [SMILES: *CCCCN1C(=O)c2ccccc2C1=O] | 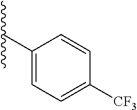 [SMILES: *c1ccc(C(F)(F)F)cc1] | 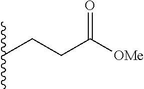 [SMILES: *CCC(=O)OC] |
| Cl | 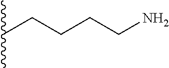 [SMILES: *CCCCN] | 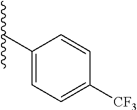 [SMILES: *c1ccc(C(F)(F)F)cc1] | 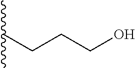 [SMILES: *CCCO] |
| Cl | Cyclohexylmethyl | 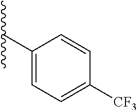 [SMILES: *c1ccc(C(F)(F)F)cc1] | 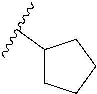 [SMILES: *C1CCCC1] |
| OH | Cbz | 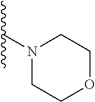 [SMILES: *N1CCOCC1] | Cbz |
| OH | Bn | 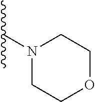 [SMILES: *N1CCOCC1] | H |
| 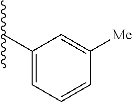 [SMILES: *c1cccc(C)c1] | Cbz | 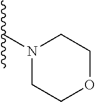 [SMILES: *N1CCOCC1] | 3,5-dichlorobenzyl |
| 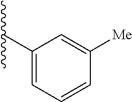 [SMILES: *c1cccc(C)c1] | Bn | 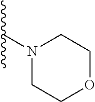 [SMILES: *N1CCOCC1] | 4-chlorobenzyl |
| 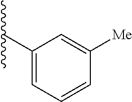 [SMILES: *c1cccc(C)c1] | H | 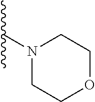 [SMILES: *N1CCOCC1] | 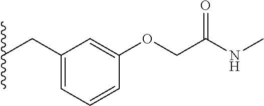 [SMILES: *Cc1cccc(OCC(=O)NC)c1] |
| Cl | Phenethyl | Cl | H |
| 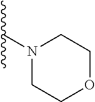 [SMILES: *N1CCOCC1] | 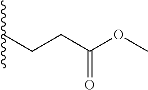 [SMILES: *CCC(=O)OC] | 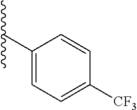 [SMILES: *c1ccc(C(F)(F)F)cc1] | 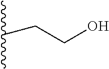 [SMILES: *CCO] |
| 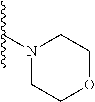 [SMILES: *N1CCOCC1] | 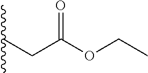 [SMILES: *CC(=O)OCC] | 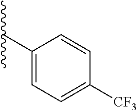 [SMILES: *c1ccc(C(F)(F)F)cc1] | 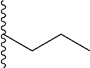 [SMILES: *CCC] |
| 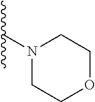 [SMILES: *N1CCOCC1] | 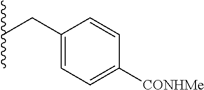 | 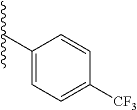 [SMILES: *c1ccc(C(F)(F)F)cc1] | 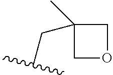 [SMILES: *CC1(C)COC1] |
| 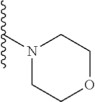 [SMILES: *N1CCOCC1] | 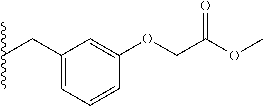 [SMILES: *Cc1cccc(OCC(=O)OC)c1] | 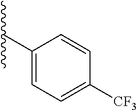 [SMILES: *c1ccc(C(F)(F)F)cc1] | 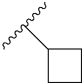 [SMILES: *C1CCC1] |
| 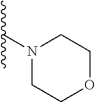 [SMILES: *N1CCOCC1] | 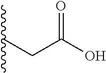 [SMILES: *CC(=O)O] | 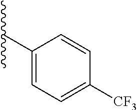 [SMILES: *c1ccc(C(F)(F)F)cc1] | 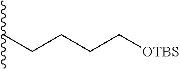 [SMILES: *CCCCO[Si](C)(C)C(C)(C)C] |
| 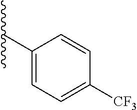 [SMILES: *c1ccc(C(F)(F)F)cc1] | 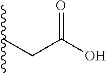 [SMILES: *CC(=O)O] | Cl | 4-chlorobenzyl |
| Cl | 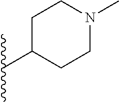 [SMILES: *C1CCN(C)CC1] | Cl | 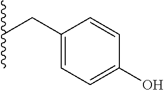 [SMILES: *Cc1ccc(O)cc1] |
| Cl | 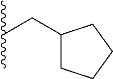 [SMILES: *CC1CCCC1] | 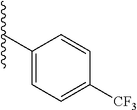 [SMILES: *c1ccc(C(F)(F)F)cc1] | 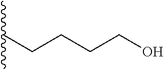 [SMILES: *CCCCO] |
| Cl | Bn | Cl | H |
| 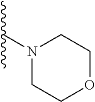 [SMILES: *N1CCOCC1] | 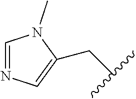 [SMILES: *Cc1cncn1C] | 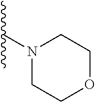 [SMILES: *N1CCOCC1] | 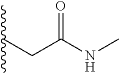 [SMILES: *CC(=O)NC] |
| 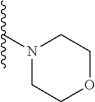 [SMILES: *N1CCOCC1] | 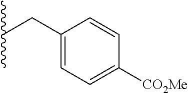 [SMILES: *Cc1ccc(C(=O)OC)cc1] | | |
| Cl | 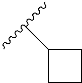 [SMILES: *C1CCC1] | | |
| Cl | Me | | |

### II. Pharmaceutical Compositions

Provided herein are pharmaceutical compositions of the compounds herein. In one aspect is a pharmaceutical composition that includes a compound described herein and a pharmaceutically acceptable excipient. In another aspect is a pharmaceutical compositions that includes a compound described herein and a pharmaceutically acceptable excipient or a pharmaceutically acceptable salt. The compound may have formula (I) as described herein. The compound may have formula (II) as described herein. The compound may have formula (III) as described herein. The compound may have formula (IV) as described herein. The compound may have formula (V) as described herein. The compound may have formula (VI) as described herein. The compound may have formula (VII) as described herein. The compound may be a compound set forth in Table A, Table B, or Table C.

The pharmaceutical composition may include a second agent in a therapeutically effective amount. The pharmaceutical composition may include a second agent where the second agent treats cancer. The second agent may be an anti-cancer agent as described herein. The pharmaceutical composition may include a second agent where the second agent treats a neurodegenerative disease (e.g. Alzheimer's Disease or ALS). The pharmaceutical composition may include a second agent where the second agent treats alcohol withdrawal. The pharmaceutical composition may include a second agent where the second agent treats depression or anxiety. The pharmaceutical composition may include a second agent where the second agent treats neuropathic pain.

1. Formulations

The pharmaceutical composition may be prepared and administered in a wide variety of dosage formulations. Compounds described herein (e.g. formula (I), (II), (III), (IV), (V), (VI), (VII) or (A)-(O)) may be administered orally, rectally, or by injection (e.g. intravenously, intramuscularly, intracutaneously, subcutaneously, intraduodenally, or intraperitoneally).

For preparing pharmaceutical compositions from compounds described herein, pharmaceutically acceptable carriers can be either solid or liquid. Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material.

In powders, the carrier may be a finely divided solid in a mixture with the finely divided active component. In tablets, the active component may be mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

2. Effective Dosages

The pharmaceutical composition may include compositions wherein the active ingredient is contained in a therapeutically effective amount, i.e., in an amount effective to achieve its intended purpose. The actual amount effective for a particular application will depend, inter alia, on the condition being treated.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

For any compound described herein or combination thereof, the therapeutically effective amounts can be initially determined from cell culture assays. Target concentrations will be those concentrations of active compound(s) that are capable of increasing the extent of cancer cell death as measured, for example, using methods known in the art.

Therapeutically effective amounts for use in humans may be determined from animal models. For example, a dose for humans can be formulated to achieve a concentration that has been found to be effective in animals. The dosage in humans can be adjusted by monitoring response of the cancer to the treatment and adjusting the dosage upwards or downwards, as described above.

Dosages may be varied depending upon the requirements of the subject and the compound being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

3. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch.1, p.1, 1975. The exact formulation, route of administration, and dosage can be chosen by the individual physician in view of the patient's condition and the particular method in which the compound is used.

When parenteral application is needed or desired, particularly suitable admixtures for the compounds included in the pharmaceutical composition may be injectable, sterile solutions, oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. Pharmaceutical admixtures suitable for use in the pharmaceutical compositions presented herein may include those described, for example, in Pharmaceutical Sciences (17th Ed., Mack Pub. Co., Easton, Pa.) and WO 96/05309, the teachings of both of which are hereby incorporated by reference.

### III. Method of Treatment

Further provided herein are methods of treating a disease in a subject in need thereof. In one aspect, is a method of treating cancer in a subject in need thereof, by administering an effective amount of a compound described herein. The cancer may be breast cancer, triple-negative breast cancer, ovarian cancer, lung cancer, prostate cancer, or skin cancer. The cancer may be breast cancer. The cancer may be triple-negative breast cancer. The cancer may be ovarian cancer. The cancer may be lung cancer. The cancer may be prostate cancer. The cancer may be skin cancer. The method may include co-administering the compounds described herein with another active pharmaceutical agent as described herein. The compound may be a compound having formula (I). The compound may be a compound having formula (VII).

In another aspect is a method of treating neurodegenerative disease in a subject in need thereof by administering an effective amount of a compound described herein. The neurodegenerative disease may be Alzheimer's disease or Amyotrophic lateral sclerosis (ALS). The neurodegenerative disease may be Alzheimer's disease. The neurodegenerative disease may be Amyotrophic lateral sclerosis (ALS). The method may include co-administering the compounds described herein with another active pharmaceutical agent as described herein. The compound may have formula:

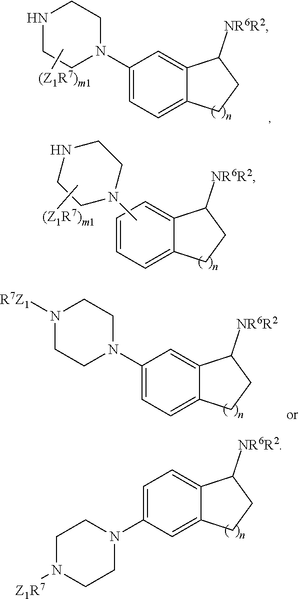

R², R⁶, R⁷, and n are as described herein.

In yet another aspect is a method of treating ethanol withdrawal in a subject in need thereof by administering an effective amount of a compound described herein. The method may include co-administering the compounds described herein with another active pharmaceutical agent as described herein. The compound may have formula:

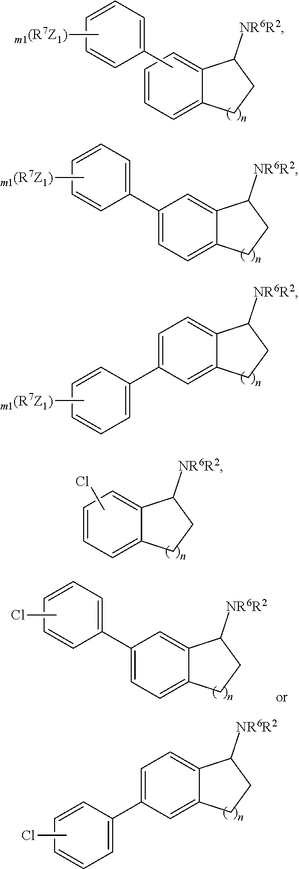

R², R⁶, R⁷, n, and m1 are as described herein. R² may be —C(O)OR⁴, hydroxyethyl, hydroxypropyl, or hydroxybutyl.

The compound for treating ethanol withdrawal may have the formula:

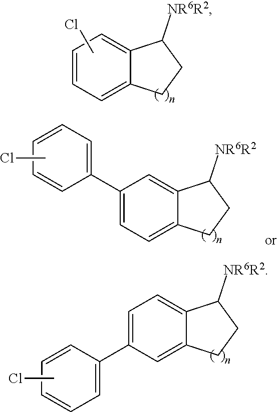

R², R⁶, R⁷, n, and m1 are as described herein. R² may be —C(O)OR⁴, hydroxyethyl, hydroxypropyl, or hydroxybutyl.

In still another aspect is a method of treating anxiety or depression in a subject in need thereof by administering an effective amount of a compound described herein. The method may include co-administering the compounds described herein with another active pharmaceutical agent as described herein.

In another aspect is a method of treating neuropathic pain in a subject in need thereof by administering an effective amount of a compound described herein.

In still yet another aspect is methods of using the compounds described herein such as those in formula I to treat a traumatic brain injury. The traumatic brain injury may be the result of an external pressure, blow, or strike to the head which results in damage to the brain with or without visible penetration of the skull. The compounds used to treat the traumatic brain injury include those which show enhanced activity against a sigma 2 receptor relative to a sigma 1 receptor, those which show enhanced activity against a sigma 1 receptor relative to a sigma 2 receptor, and those which show similar activity. In particular, it is also contemplated that the compounds used herein may be combined with one or more known therapeutic agents to form a combination therapy. The traumatic brain injury may result from a primary or a secondary injury.

### IV. Methods of Inhibiting Sigma Receptors

Provided herein are methods of inhibiting or antagonizing a sigma 2 receptor by contacting a sigma 2 receptor with a compound described herein, thereby inhibiting the sigma 2 receptor. The compound may have the formula:

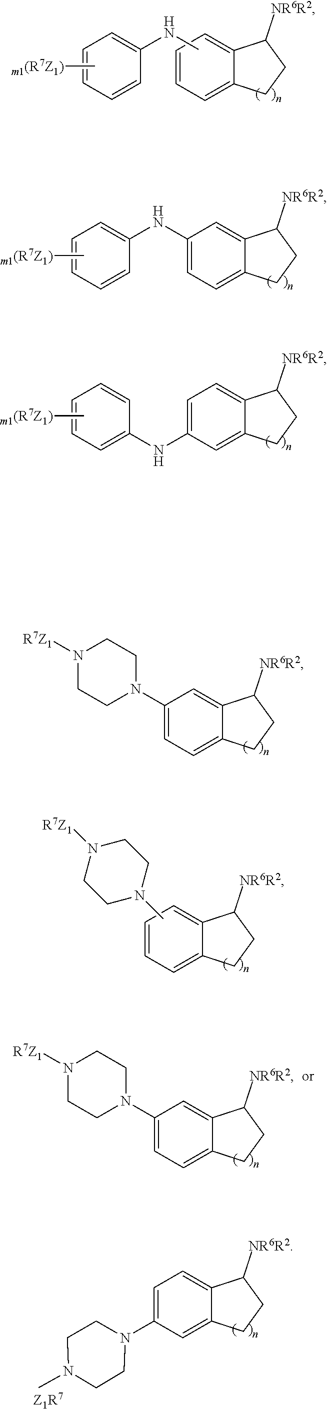

R², R⁶, R⁷, n, and m1 are as described herein.

In another aspect is a method of inhibiting a sigma 1 receptor by contacting a sigma 1 receptor with a compound described herein. The compound may have the structure:

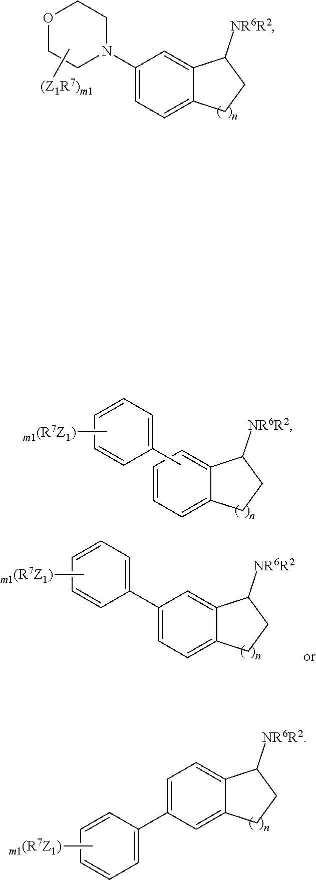

R², R⁶, R⁷, n, and m1 are as described herein.

### V. Methods of Activating Sigma Receptors

Provided herein are methods of activating or agonizing a sigma 2 receptor by contacting a sigma 2 receptor with a compound described herein, thereby activating the sigma 2 receptor. The compound may have the formula:

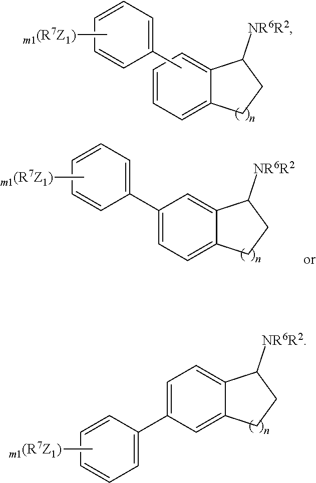

R², R⁶, R⁷, n, and m1 are as described herein.

In another aspect is a method of activating a sigma 1 receptor by contacting a sigma 1 receptor with a compound described herein, thereby activating the sigma 1 receptor. The compound may have the structure:

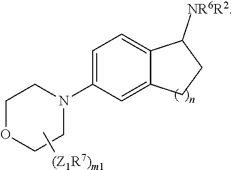

R², R⁶, R⁷, n, and m1 are as described herein.

### VI. Examples

### 1. Examples 1

TABLE 1
Sigma Receptor Affinity of 7-Piperazinyl-tetrahydronaphthalene Analogs.

| | | | Ki (nM) | | Ki ratio |
|---|---|---|---|---|---|
| Compound | R¹ | R² | Sig1R | Sig2R | Sig1R/Sig2R |
| MDW-1-93 | 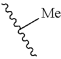 | 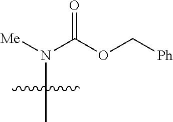 [SMILES: *N(C)C(=O)OCc1ccccc1] | 2,944 | 284 | 10 |
| MDW-1-161 | 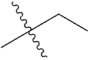 [SMILES: *CC] | 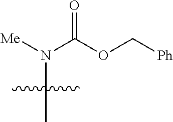 [SMILES: *N(C)C(=O)OCc1ccccc1] | 887 | 35 | 25 |
| MDW-1-167 | 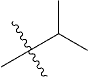 [SMILES: *C(C)C] | 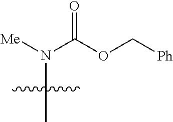 [SMILES: *N(C)C(=O)OCc1ccccc1] | 223 | 8 | 27 |
| MDW-1-202 | 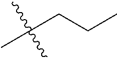 [SMILES: *CCC] | 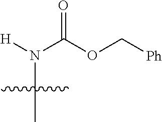 [SMILES: *NC(=O)OCc1ccccc1] | 174 | 56 | 3 |
| MDW-2-76 | 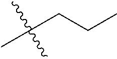 [SMILES: *CCC] | 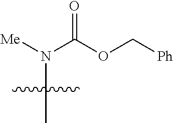 [SMILES: *N(C)C(=O)OCc1ccccc1] | 200 | 6 | 36 |
| MDW-1-195 | 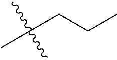 [SMILES: *CCC] | 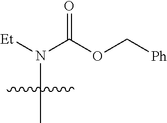 [SMILES: *N(CC)C(=O)OCc1ccccc1] | 321 | 5 | 70 |
| MDW-1-196 | 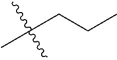 [SMILES: *CCC] | 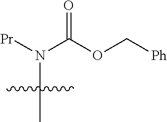 | 199 | 9 | 22 |
| MDW-1-236 | 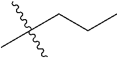 [SMILES: *CCC] | 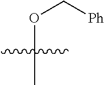 [SMILES: *OCc1ccccc1] | 104 | 50 | 2 |
| MDW-1-290 | 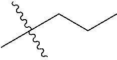 [SMILES: *CCC] | 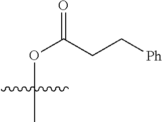 [SMILES: *OC(=O)CCc1ccccc1] | 33 | 25 | 1 |
| MDW-1-162 | 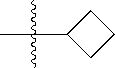 [SMILES: *C1CCC1] | 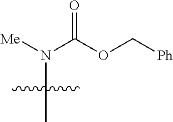 [SMILES: *N(C)C(=O)OCc1ccccc1] | 266 | 12 | 22 |
| MDW-1-140 | 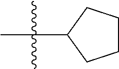 [SMILES: *C1CCCC1] | 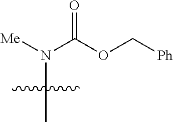 [SMILES: *N(C)C(=O)OCc1ccccc1] | 86 | 2 | 41 |
| MDW-1-205 | 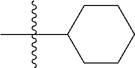 [SMILES: *C1CCCCC1] | 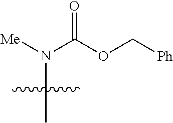 [SMILES: *N(C)C(=O)OCc1ccccc1] | 16 | 2 | 8 |
| MDW-1-206 | 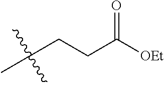 [SMILES: *CCC(=O)OCC] | 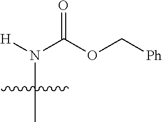 [SMILES: *NC(=O)OCc1ccccc1] | 2,256 | 80 | 28 |
| MDW-1-208 | 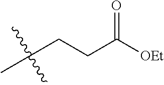 [SMILES: *CCC(=O)OCC] | 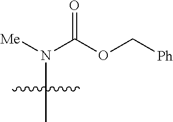 [SMILES: *N(C)C(=O)OCc1ccccc1] | 1,649 | 5 | 366 |
| MDW-1-190 | 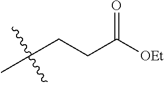 [SMILES: *CCC(=O)OCC] | 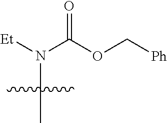 [SMILES: *N(CC)C(=O)OCc1ccccc1] | 3,315 | 20 | 166 |
| MDW-1-191 | 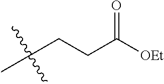 [SMILES: *CCC(=O)OCC] | 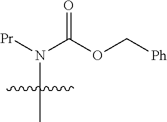 | 2,364 | 19 | 124 |
| MDW-1-169 | 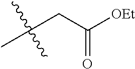 [SMILES: *CC(=O)OCC] | 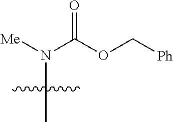 [SMILES: *N(C)C(=O)OCc1ccccc1] | >10,000 | 894 | — |
| MDW-1-166 | 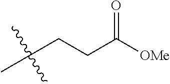 [SMILES: *CCC(=O)OC] | 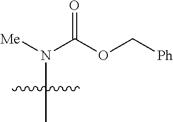 [SMILES: *N(C)C(=O)OCc1ccccc1] | 2,864 | 43 | 67 |
| MDW-1-213 | 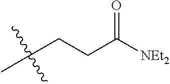 | 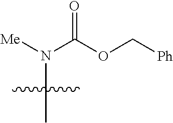 [SMILES: *N(C)C(=O)OCc1ccccc1] | 2,101 | 7 | 292 |
| MDW-1-177 | 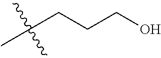 [SMILES: *CCCO] | 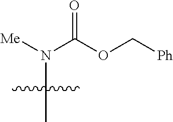 [SMILES: *N(C)C(=O)OCc1ccccc1] | 474 | 21 | 23 |

TABLE 2
Sigma Receptor Affinity of Piperazinyl-tetrahydronaphthalene Analogs.

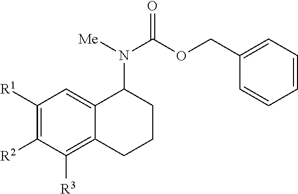

| | | | | Ki (nM) | | Ki ratio |
|---|---|---|---|---|---|---|
| Compound | R¹ | R² | R³ | Sig1R | Sig2R | Sig1R/Sig2R. |
| MDW-1-204 | H | 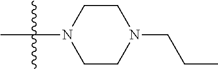 [SMILES: *N1CCN(CCC)CC1] | H | 40 | 13 | 3 |
| MDW-1-215 | H | H | 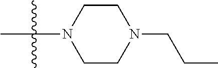 [SMILES: *N1CCN(CCC)CC1] | 12 | 26 | 0.5 |
| MDW-1-207 | H | 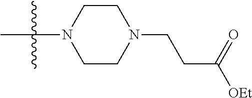 [SMILES: *N1CCN(CCC(=O)OCC)CC1] | H | 647 | 91 | 7 |
| MDW-1-214 | H | H | 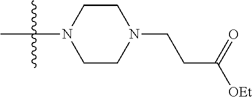 [SMILES: *N1CCN(CCC(=O)OCC)CC1] | 303 | 34 | 9 |

Table 3. Sigma Receptor Affinity of Aminotetralin analogs.

### Experimental

General methods. Methylene chloride (CH₂Cl₂) was distilled from calcium hydride (CaH₂) immediately prior to use. All solvents were determined to have less than 50 ppm H₂O by Karl Fischer coulometric moisture analysis. All reagents were reagent grade and used without purification unless otherwise noted. N,N-Diisopropylethylamine (DIPEA), Morpholine, benzylbromide, tBuOH, and 1-methylpiperazine were distilled from CaH₂ prior to use. All reactions involving air or moisture sensitive reagents or intermediates were performed under an inert atmosphere of nitrogen or argon in glassware that was flame dried. Reaction temperatures refer to the temperature of the cooling/heating bath. Volatile solvents were removed under reduced pressure using a Büchi rotary evaporator. Thin-layer chromatography (TLC) was performed on EMD 60 F254 glass-backed pre-coated silica gel plates and were visualized using one or more of the following methods: UV light (254 nm) and staining with basic potassium permanganate (KMnO4) or acidic p-anisaldehyde (PAA). Infrared (IR) spectra were obtained with a Thermo Scientific Nicolet IR-100 FT-IR series spectrometer as thin films on sodium chloride plates and reported in wavenumbers (cm⁻¹). Melting points were determined using a Thomas-Hoover Uni-melt capillary melting point apparatus. Proton nuclear magnetic resonance (¹H NMR) and carbon nuclear magnetic resonance (¹³C NMR) spectra were obtained at the indicated field as solutions in CDCl₃ unless otherwise indicated. Chemical shifts are referenced to the deuterated solvent and are reported in parts per million (ppm, δ) downfield from tetramethylsilane (TMS, δ=0.00 ppm). Coupling constants (J) are reported in Hz and the splitting abbreviations used are: s, singlet; d, doublet; t, triplet; q, quartet; m, multiplet; comp, overlapping multiplets of magnetically nonequivalent protons; br, broad; app, apparent.

### Representative Procedure: Buchwald-Hartwig Cross Coupling with JohnPhos©

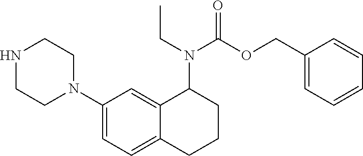 [SMILES: CCN(C(=O)OCc1ccccc1)C1CCCc2ccc(N3CCNCC3)cc21]

Benzyl ethyl(7-(piperazin-1-yl)-1,2,3,4-tetrahydronaphthalen-1-yl)carbamate (MDW-1-185). A sealable tube was charged with a solution containing carbamate MDW-1-182 (0.0.432 g, 1.11 mmol), NaOtBu (0.161 g, 1.67 mmol), and piperazine (0.480 g, 5.57 mmol) in degassed toluene (2.2 mL). A freshly prepared solution of Pd(OAc)₂ and di-tert-butylphosphine biphenyl (JohnPhos©) (1:1, 0.7 mL, 0.08 M), that had been stirred for 30 min, was added. The tube was sealed and the reaction was stirred at 100° C. for 7 h. The reaction mixture was then filtered through Celite®, washing the filter cake with CH₂Cl₂ (100 mL), and the filtrate was concentrated. The residue was dissolved in CH₂Cl₂ (20 mL), washed with 1 N NaOH (1×20 mL), dried (Na₂SO₄) and concentrated under reduced pressure. The crude residue was purified via flash chromatography (SiO₂) eluting with DCM/MeOH/Et₃N (97:2:1) to give 0.320 g (73%) of MDW-1-185 as an orange oil. ¹H NMR (400 MHz, CDCl₃) mixture of rotamers: δ 7.41-7.17 (comp, 5H), 6.93 (d, J=8.4 Hz, 1H), 6.70 (dd, J=8.4, 2.6 Hz, 1H), 6.57 (brs, 1H), 5.46-4.97 (comp, 4H), 3.35-3.06 (comp, 2H), 2.98-2.86 (comp, 8H), 2.71-2.56 (comp, 2H), 2.07-1.94 (comp, 2H), 1.85-1.59 (comp, 2H), 1.15 and 1.09 (t, J=7.0 Hz, 3H). HRMS (ESI) m/z calcd for C₂₄H₃₁N₃O₂ (M+H)⁺, 394.2489; found 394.2507.

### Representative Procedure: Conjugate Addition to Ethyl Acrylate

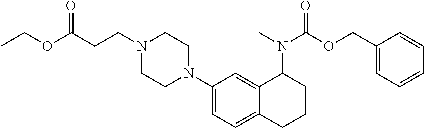 [SMILES: CCOC(=O)CCN1CCN(c2ccc3c(c2)C(N(C)C(=O)OCc2ccccc2)CCC3)CC1]

Ethyl 3-(4-(8-(((benzyloxy)carbonyl)(methyl)amino)-5,6,7,8-tetrahydronaphthalen-2-yl)piperazin-1-yl)propanoate (MDW-2-120). A solution of amine MDW-2-111 (0.0831 g, 0.219 mmol) and ethyl acrylate (0.22 g, 0.24 mL, 2.2 mmol) in EtOH (1.1 mL) was stirred at 40° C. for 18 h. The reaction was concentrated under reduce pressure and the crude residue was purified via flash chromatography (SiO₂) eluting with Hexanes/EtOAc/Et₃N (64:35:1) to afford 0.0902 g (86%) of MDW-2-120 as a clear oil. ¹H NMR (400 MHz, CDCl₃) mixture of rotamers: δ 7.46-7.28 (comp, 5H), 6.99 (d, J=8.2 Hz, 1H), 6.79-6.73 (m, 1H), 6.61 (dd, J=10.8, 2.2 Hz, 1H), 5.51-5.44 and 5.38-5.32 (m, 1H), 5.31-5.11 (comp, 2H), 4.16 (q, J=7.1 Hz, 2H), 3.12-3.02 (comp, 4H), 2.76 (t, J=7.4 Hz, 2H), 2.71-2.63 (comp, 5H), 2.62-2.57 (comp, 4H), 2.54 (t, J=7.4 Hz, 2H), 2.07-1.92 (comp, 2H), 1.83-1.66 (m, 2H), 1.27 (app t, J=7.2 Hz, 3H). HRMS (ESI) m/z calcd for C₂₈H₃₇N₃O₄ (M+H)⁺, 480.2857; found 480.2857.

### Representative Procedure: Reductive Amination with an Aldehyde

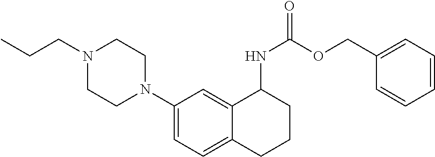 [SMILES: CCCN1CCN(c2ccc3c(c2)C(NC(=O)OCc2ccccc2)CCC3)CC1]

Benzyl (7-(4-propylpiperazin-1-yl)-1,2,3,4-tetrahydronaphthalen-1-yl)carbamate (MDW-1-202). Propionaldehyde (0.014 g, 0.018 mL, 0.25 mmol), was added to a solution of amine MDW-1-197 (0.030 g 0.082 mmol), Na(OAc)₃BH (0.035 g, 0.17 mmol), in DCE (0.82 mL). The solution was stirred at room temperature for 23 h, diluted with CH₂Cl₂ (10 mL) and partitioned between saturated aq. NaHCO₃ (10 mL). The organic layer was separated and the aqueous layer was extracted with CH₂Cl₂ (2×10 mL). The combined organic extracts were dried (Na₂SO₄), and concentrated under reduced pressure. The crude residue was purified via flash chromatography (SiO₂) eluting with Hexanes/EtOAc/Et₃N (74:25:1) to give 0.0282 g (84%) of MDW-1-202 as a white solid. ¹H NMR NMR (499 MHz, CDCl₃) δ 7.42-7.30 (comp, 5H), 6.98 (d, J=8.4 Hz, 1H), 6.88 (d, J=2.6 Hz, 1H), 6.79 (dd, J=8.4, 2.6 Hz, 1H), 5.19-5.11 (m, 2H), 5.01 (d, J=8.7 Hz, 1H), 4.90-4.84 (m, 1H), 3.19-3.08 (comp, 4H), 2.75-2.63 (comp, 2H), 2.62-2.56 (comp, 4H), 2.40-2.33 (comp, 2H), 2.06-1.97 (m, 1H), 1.88-1.74 (comp, 3H), 1.61-1.51 (comp, 2H), 0.94 (t, J=7.4 Hz, 3H). HRMS (ESI) m/z calcd for C₂₅H₃₃N₃O₂ (M+H)⁺, 408.2646; found 408.2662.

### Representative Procedure: Reductive Amination with Ketone

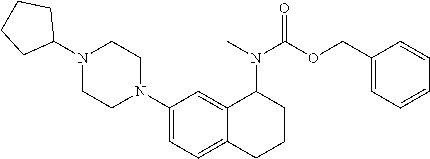 [SMILES: CN(C(=O)OCc1ccccc1)C1CCCc2ccc(N3CCN(C4CCCC4)CC3)cc21]

Benzyl (7-(4-cyclopentylpiperazin-1-yl)-1,2,3,4-tetrahydronaphthalen-1-yl)(methyl)carbamate (MDW-1-140). Cyclopentanone (0.013 g, 0.014 mL, 0.16 mmol), was added to a solution of amine MDW-2-111 (0.020 g 0.053 mmol), Na(OAc)₃BH (0.0232 g, 0.109 mmol), and CH₃CO₂H (0.005 mL) in DCE (1.1 mL). The solution was stirred at room temperature for 9 h, diluted with CH₂Cl₂ (10 mL) and partitioned between saturated aq. NaHCO₃ (10 mL). The organic layer was separated and the aqueous layer was extracted with CH₂Cl₂ (2×10 mL). The combined organic extracts were dried (Na₂SO₄), and concentrated under reduced pressure. The crude residue was purified via flash chromatography (SiO₂) eluting with Hexanes/EtOAc/Et₃N (74:25:1) to give 0.0217 g (92%) of MDW-1-140 as a clear oil. ¹H NMR (499 MHz, CDCl3) rotamers δ 7.44-7.27 (comp, 5H), 6.99 (d, J=8.3 Hz, 1H), 6.79-6.74 (m, 1H), 6.62 (dd, J=7.7, 2.5 Hz, 1H), 5.49-5.32 (m, 1H), 5.28-5.11 (comp, 2H), 3.16-3.05 (comp, 4H), 2.72-2.60 (comp, 9H), 2.55 (t, J=8.3 Hz, 1H), 2.05-1.88 (comp, 4H), 1.81-1.68 (comp, 4H), 1.63-1.53 (comp, 2H), 1.52-1.41 (comp, 2H). HRMS (ESI) m/z calcd for C₂₈H₃₇N₃O₂ (M+H)⁺, 448.2959; found 448.2962.

### Representative Procedure: Alkylation of Amine with Alkyl Halide

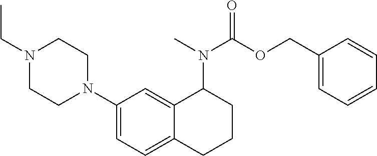 [SMILES: CCN1CCN(c2ccc3c(c2)C(N(C)C(=O)OCc2ccccc2)CCC3)CC1]

Benzyl (7-(4-ethylpiperazin-1-yl)-1,2,3,4-tetrahydronaphthalen-1-yl)(methyl)carbamate (MDW-1-161). A solution of amine MDW-2-111 (0.030 g, 0.079 mmol), bromoethane (0.010 g, 0.007 mL, 0.095 mmol), and K₂CO₃ (022 g, 0.16 mmol) in MeCN (0.8 mL) was stirred at room temperature for 24 hours. The solution was diluted with CH₂Cl₂ (10 mL) and partitioned between 1 N NaOH (1×10 mL). The organic layer was separated and the aqueous was extracted with CH₂Cl₂ (2×10 mL). The combined organic extracts were dried (Na₂SO₄), and concentrated under reduced pressure. The crude residue was purified via flash chromatography (SiO₂) eluting with Et₂O/Hexanes/Et₃N (69:30:1) affording 0.023 g (70%) of MDW-1-161 as a clear oil. ¹H NMR (499 MHz, CDCl₃) rotamers δ 7.45-7.28 (comp, 5H), 6.99 (d, J=8.3 Hz, 1H), 6.81-6.75 (m, 1H), 6.63 (dd, J=11.1, 2.6 Hz, 1H), 5.51-5.32 (m, 1 H), 5.32-5.11 (comp, 2H), 3.18-3.06 (comp, 4H), 2.72-2.56 (comp, 9H), 2.49 (q, J=7.2 Hz, 2H), 2.07-1.91 (comp, 2H), 1.84-1.69 (comp, 2H), 1.14 (t, J=7.2 Hz, 3H). HRMS (ESI) m/z calcd for C₂₅H₃₃N₃O₂ (M+Na)⁺, 430.2465; found 430.2483.

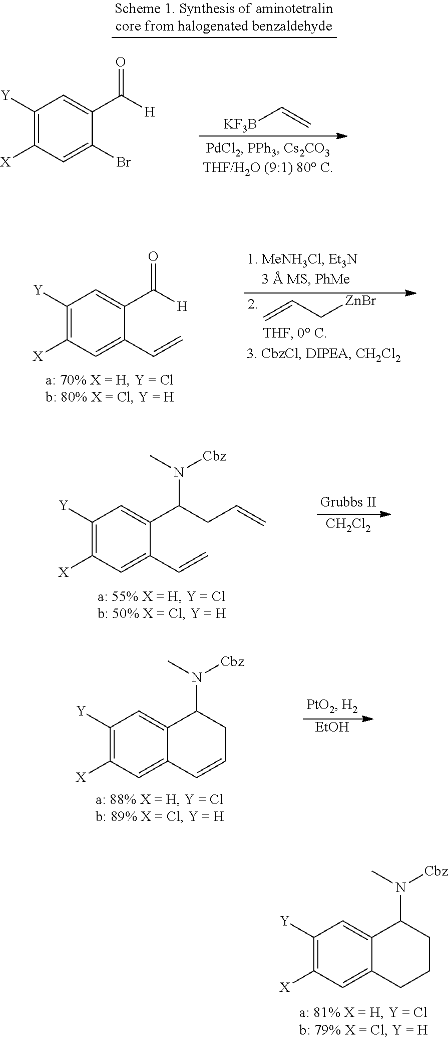

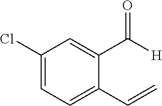

5-Chloro-2-vinylbenzaldehyde (MDW-1-75). According to a literature procedure, a resealable tube was charged with 2-bromo-5-chlorobenzaldehyde (1.800 g, 27.34 mmol), potassium vinyltrifluoroborate (1.428 g, 10.66 mmol), PdCl₂ (0.117 g, 0.660 mmol), triphenylphosphine (0.517 g, 1.97 mmol), and cesium carbonate (8.022 g, 24.62 mmol).¹ A solution of degassed THF/water (20.5 mL, 9:1) was added to the tube, whereupon it was sealed and stirred at 85° C. for 6 h. The reaction mixture was allowed to cool to room temperature, diluted with water (30 mL), and extracted using Et₂O (3×30 mL). The organic layer was washed with brine (1×30 mL), dried (Na₂SO₄), and concentrated under reduced pressure to provide an orange oil. The crude residue was purified via flash chromatography (SiO₂), eluting with 1 hexanes/EtOAc (99:1) affording 1.091 g (80%) of MDW-1-75 as a pale yellow solid. ¹H NMR consistent with those reported in literature.¹

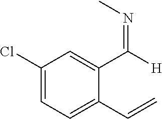

1-(5-Chloro-2-vinylphenyl)-N-methylmethanimine (MDW-1-108). A solution containing MDW-1-75 (2.25 g, 13.5 mmol), methylamine hydrochloride (9.11 g, 135 mmol), Et₃N (20.5 g, 28.2 mL, 203 mmol), and 3 Å powdered sieves (11.2 g) in dry toluene (54 mL) was stirred for 44 h. The suspension was filtered through a pad of Celite®, and the filter cake was washed with toluene (100 mL). The filtrate was concentrated under reduced pressure affording 2.25 g (93%) of MDW-1-108 as an orange oil that was used without purification. ¹H NMR (400 MHz, CDCl₃) δ 8.46-8.41 (m, 1H), 7.78 (d, J=2.3 Hz, 1H), 7.29 (d, J=8.4 Hz, 1H), 7.22 (dd, J=8.4, 2.3 Hz, 1H), 7.05 (dd, J=17.4, 11.0 Hz, 1H), 5.53 (dd, J=17.3, 1.2 Hz, 1H), 5.34 (dd, J=11.0, 1.2 Hz, 1H), 3.45 (d, J=1.7 Hz, 3H).

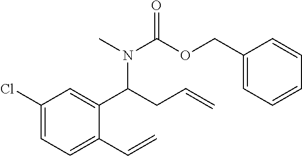 [SMILES: C=CCC(c1cc(Cl)ccc1C=C)N(C)C(=O)OCc1ccccc1]

Benzyl (1-(5-chloro-2-vinylphenyl)but-3-en-1-yl)(methyl)carbamate (MDW-1-154). A solution of allylzinc bromide in THF (1.5 M, 4.6 mL, 6.89 mmol) was added dropwise to a solution of imine MDW-1-108 (0.952 g, 5.23) in THF (26.5 mL) cooled to −78° C. The solution was stirred at −78° C. for 5 h, warmed to 0° C. and quenched with water (3 mL). The solution was diluted with saturated aq. NH₄Cl (5 mL) and extracted with CH₂Cl₂ (3×20 mL). The combined organic extracts were washed with brine (1×30 mL), dried (Na₂SO₄), and concentrated under reduced pressure affording. The crude residue was taken up in CH₂Cl₂ (53 mL) and cooled to 0° C., followed by sequential addition of DIPEA (1.37 g, 1.85 mL, 10.6 mmol) and CbzCl (1.09 g, 0.91 mL, 6.36 mmol). The solution was stirred for 2 h and allowed to warm to room temperature. The reaction mixture was diluted with CH₂Cl₂ (10 mL), washed with 1 N HCl (2×25 mL), 1 N NaOH (2×25 mL), brine (1×25 mL), dried (Na₂SO₄) and concentrated under reduced pressure. The crude residue was purified via flash chromatography (SiO₂) eluting with hexanes/EtOAc (95:5) to give 1.36 g (73% over 3 steps) of MDW-1-154 as a clear oil. ¹H NMR (500 MHz, 130° C., DMSO-d₆) δ 7.50 (d, J=8.3 Hz, 1H), 7.40 (d, J=2.3 Hz, 1H), 7.38-7.29 (comp, 6H), 6.94 (dd, J=17.2, 11.0 Hz, 1H), 5.81-5.70 (m, 1H), 5.59 (dd, J=17.3, 1.3 Hz, 1H), 5.47 (dd, J=8.8, 6.5 Hz, 1H), 5.22 (dd, J=11.0, 1.3 Hz, 1H), 5.16-5.09 (comp, 3H), 5.05-5.00 (m, 1H), 2.79-2.66 (m, 2H), 2.63 (s, 3H). HRMS (ESI) m/z calcd for C₂₁H₂₂ClNO₂ (M+Na)⁺, 378.1231; found 378.1232.

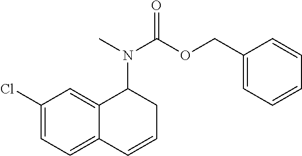 [SMILES: CN(C(=O)OCc1ccccc1)C1CC=Cc2ccc(Cl)cc21]

Benzyl (7-chloro-1,2-dihydronaphthalen-1-yl)(methyl)carbamate (MDW-1-156). A solution of MDW-1-154 (1.36 g, 3.83 mmol) and Grubbs 2^{nd} generation catalyst (0.163 g, 0.192 mmol) in DCM (77 mL) was stirred for 2 h at room temperature. DMSO (0.7 mL) was added to the solution and it was stirred for 14 h, then concentrated under reduced pressure. The crude residue was passed through a SiO₂ plug eluting with hexanes/EtOAc (85:15) and concentrated, whereupon the residue was purified via flash chromatography eluting with hexanes/EtOAc (92:8) to give 1.11 g (88%) of MDW-1-156 as a pale yellow oil oil. ¹H NMR (500 MHz, 130° C., DMSO-d₆) δ 7.39-7.35 (comp, 4H), 7.34-7.29 (m, 1H), 7.26 (ddd, J=8.1, 2.2, 0.8 Hz, 1H), 7.15 (d, J=8.1 Hz, 1H), 7.06-7.03 (m, 1H), 6.50-6.45 (m, 1H), 6.12-6.07 (m, 1H), 5.44 (t, J=9.2 Hz, 1H), 5.18 (app s, 2H), 2.77 (app s, 3H), 2.58-2.53 (m, 2H). HRMS (ESI) m/z calcd for C₁₉H₁₈ClNO₂ (M+Na)⁺, 350.0918; found 350.0922.

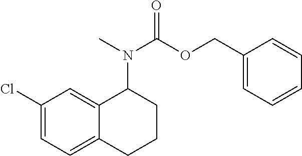 [SMILES: CN(C(=O)OCc1ccccc1)C1CCCc2ccc(Cl)cc21]

Benzyl (7-chloro-1,2,3,4-tetrahydronaphthalen-1-yl)(methyl)carbamate (MDW-1-158). H₂ gas was bubbled through a solution containing carbamate MDW-1-156 (0.300 g, 0.915 mmol) and PtO₂ (0.0103 g, 0.005 mmol) in EtOH (9.2 mL) for 5 min. The solution was stirred under a hydrogen atmosphere (1 atm) for 2 h, filtered through Celite® washing the filter cake with CH₂Cl₂ (100 mL), and concentrated under reduced pressure. The crude residue was purified via flash chromatography (SiO₂) eluting with hexanes/EtOAc (4:1) to give 0.240 g (78%) of MDW-1-158 as a clear oil. ¹H NMR (499 MHz, CDCl₃) δ 7.49-7.27 (comp, 5H), 7.14-7.10 (comp, 2H), 7.05-7.00 (m, 1H), 5.53-5.30 (m, 1H), 5.29-5.20 (comp, 2H), 2.80-2.66 (comp, 5H), 2.10-1.95 (comp, 2H), 1.86-1.69 (comp, 2H). HRMS (ESI) m/z calcd for C₁₉H₂₀ClNO₂ (M+Na)⁺, 352.1075; found 352.1079.

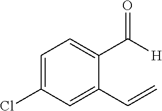

4-Chloro-2-vinylbenzaldehyde (MDW-1-106). In a modification of a literature procedure, a resealable tube was charged with 2-bromo-4-chlorobenzaldehyde (6.0 g, 27.34 mmol), potassium vinyltrifluoroborate (4.76 g, 35.54 mmol), PdCl₂ (0.388 g, 2.19 mmol), triphenylphosphine (1.72 g, 6.56 mmol), and cesium carbonate (26.72 g, 82.02 mmol).¹ A solution of THF/water (150 mL, 9:1) was added to the tube, whereupon it was sealed and stirred at 85° C. for 6 h. The reaction mixture was allowed to cool to room temperature, diluted with water (30 mL), and extracted using Et₂O (3×30 mL). The organic layer was washed with brine (1×30 mL), dried (Na₂SO₄), and concentrated under reduced pressure to provide an orange oil. The crude residue was purified via flash chromatography (SiO₂), eluting with 1 hexanes/EtOAc (99:1) affording 3.18 g (70%) of MDW-1-106 as a pale yellow solid. ¹H NMR (400 MHz, CDCl₃) δ 10.07 (s, 1H), 7.59 (d, J=8.3 Hz, 1H), 7.39-7.30 (m, 2H), 7.21 (dd, J=8.3, 2.1 Hz, 1H), 5.58 (dd, J=17.4, 1.0 Hz, 1H), 5.41 (dd, J=11.0, 1.0 Hz, 1H).

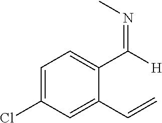

1-(4-chloro-2-vinylphenyl)-N-methylmethanimine (MDW-1-109). A solution containing MDW-1-106 (3.01 g, 18.1 mmol), methylamine hydrochloride (12.2 g, 181 mmol), Et₃N (27.4 g, 37.7 mL, 270 mmol), and 3 Å powdered sieves (15 g) in dry toluene (72 mL) was stirred for 44 h. The suspension was filtered through a pad of Celite®, and the filter cake was washed with toluene (100 mL). The filtrate was concentrated under reduced pressure affording 3.01 g (93%) of MDW-1-108 as an orange oil that was used without purification. ¹H NMR (400 MHz, CDCl₃) δ 8.39-8.34 (m, 1H), 7.66 (d, J=8.4 Hz, 1H), 7.31 (d, J=2.2 Hz, 1H), 7.13 (dd, J=8.4, 2.2 Hz, 1H), 7.05 (dd, J=17.3, 11.0 Hz, 1H), 5.51 (dd, J=17.3, 1.1 Hz, 1H), 5.30 (dd, J=11.0, 1.1 Hz, 1H), 3.39 (d, J=1.8 Hz, 3H).

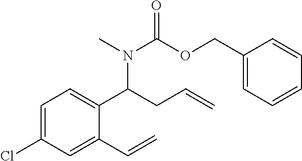 [SMILES: C=CCC(c1ccc(Cl)cc1C=C)N(C)C(=O)OCc1ccccc1]

Benzyl (1-(4-chloro-2-vinylphenyl)but-3-en-1-yl)(methyl)carbamate (MDW-1-117). A solution of allylzinc bromide in THF (1.2 M, 13.6 mL, 16.7 mmol) was added dropwise to a solution of imine MDW-1-109 (1.50 g, 8.35) in THF (33 mL) cooled to −78° C. The solution was stirred at −78° C. for 7 h, warmed to 0° C. and quenched with water (3 mL). The solution was diluted with saturated aq. NH₄Cl (5 mL) and extracted with CH₂Cl₂ (3×20 mL). The combined organic extracts were washed with brine (1×30 mL), dried (Na₂SO₄), and concentrated under reduced pressure affording. The crude residue was taken up in CH₂Cl₂ (84 mL) and cooled to 0° C., followed by sequential addition of DIPEA (3.26 g, 4.4 mL, 25.3 mmol) and CbzCl (2.15 g, 1.80 mL, 12.6 mmol). The solution was stirred for 2 h and allowed to warm to room temperature. The reaction mixture was diluted with CH₂Cl₂ (10 mL), washed with 1 N HCl (2×25 mL), 1 N NaOH (2×25 mL), brine (1×25 mL), dried (Na₂SO₄) and concentrated under reduced pressure. The crude residue was purified via flash chromatography (SiO₂) eluting with hexanes/EtOAc (97:3) to give 1.36 g (39% over 3 steps) of MDW-1-117 as a clear oil. ¹H NMR (500 MHz, 80° C., DMSO-d₆) δ 7.53 (d, J=2.3 Hz, 1H), 7.44 (d, J=8.4 Hz, 1H), 7.41-7.29 (comp, 6H), 6.93 (dd, J=17.2, 11.0 Hz, 1H), 5.79-5.71 (m, 1H), 5.68 (dd, J=17.2, 1.2 Hz, 1H), 5.51-5.45 (m, 1H), 5.23 (d, J=11.0 Hz, 1H), 5.16-5.09 (comp, 3H), 5.03-4.98 (m, 1H), 2.77-2.62 (comp, 2H), 2.55 (s, 3H). HRMS (ESI) m/z calcd for C₂₁H₂₂ClNO₂ (M+Na)⁺, 378.1231; found 378.1237.

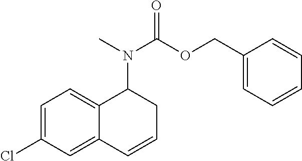 [SMILES: CN(C(=O)OCc1ccccc1)C1CC=Cc2cc(Cl)ccc21]

Benzyl (6-chloro-1,2-dihydronaphthalen-1-yl)(methyl)carbamate (MDW-1-74). A solution of MDW-1-117 (0.163 g, 0.459 mmol) and Grubbs 2^{nd} generation catalyst (0.0196 g, 0.023 mmol) in CH₂Cl₂ (23 mL) was stirred for 5 h at room temperature. SiO₂ (1.5 mL) was added to the solution and was then filtered through Celite®, washing the filter cake with CH₂Cl₂ (100 mL). The filtrate was concentrated under reduced pressure and the crude residue was purified via flash chromatography (SiO₂) eluting with hexanes/EtOAc (92:8) to give 0.134 g (89%) of MDW-1-74 as a pale yellow oil. ¹H NMR (500 MHz, 80° C., DMSO-d₆) δ 7.40-7.30 (comp, 5H), 7.25-7.21 (comp, 2H), 7.06 (d, J=7.9 Hz, 2H), 6.48 (dt, J=9.7, 2.0 Hz, 1H), 6.17-6.12 (m, 1H), 5.43 (t, J=9.2 Hz, 1H), 5.15 (s, 2H), 2.73 (s, 3H), 2.57-2.52 (comp, 2H).

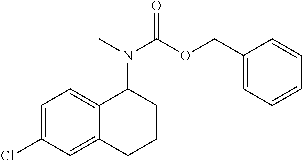 [SMILES: CN(C(=O)OCc1ccccc1)C1CCCc2cc(Cl)ccc21]

Benzyl (6-chloro-1,2,3,4-tetrahydronaphthalen-1-yl)(methyl)carbamate (MDW-1-121). H₂ gas was bubbled through a solution containing carbamate MDW-1-74 (0.639 g, 1.95 mmol) and PtO₂ (0.022 g, 0.097 mmol) in EtOH (20 mL) for 5 min. The solution was stirred under a hydrogen atmosphere (1 atm) for 1 h, filtered through Celite® washing the filter cake with CH₂Cl₂ (100 mL), and concentrated under reduced pressure. The crude residue was purified via flash chromatography (SiO₂) eluting with hexanes/EtOAc (4:1) to give 0.575 g (89%) of MDW-1-121 as a clear oil. ¹H NMR (500 MHz, 130° C., DMSO-d₆) δ 7.40-7.29 (comp, 5H), 7.17-7.14 (comp, 2H), 7.04-7.00 (m, 1H), 5.26-5.21 (m, 1H), 5.17 (s, 2H), 2.81-2.68 (comp, 2H), 2.65 (br s, 3H). HRMS (ESI) m/z calcd for C₁₉H₂₀ClNO₂ (M+Na)⁺, 352.1075; found 352.1082.

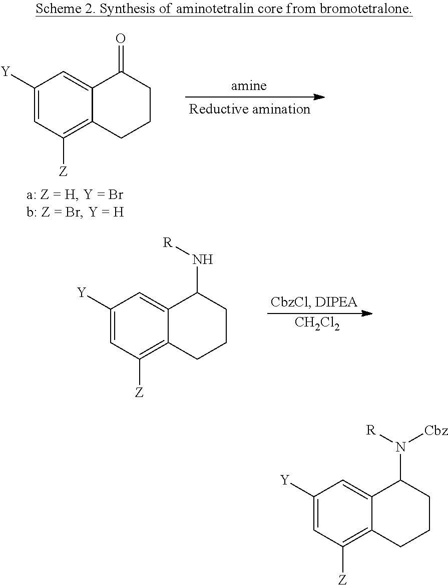

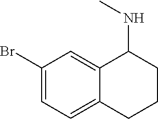

7-Bromo-N-methyl-1,2,3,4-tetrahydronaphthalen-1-amine (MDW-2-124). In a modification of a literature procedure, 7-bromotetralone (1.50 g, 6.68 mmol) was dissolved in EtOH (13.5 mL) in a resealable tube, whereupon Ti(OiPr)₄ (4.7 g, 4.9 mL, 17 mmol), Et₃N (3.4 g, 4.7 mL, 34 mmol) and MeNH₃Cl (2.26 g, 33.5 mmol) were sequentially added.² The tube was sealed, and the reaction was stirred at room temperature for 22 h. The solution was cooled to 0° C., and NaBH₄ (0.506 g, 13.4 mmol) was added in one portion. Stirring was continued at 0° C. for 1 h, and the mixture was added to 2 M aq. NH₄OH (20 mL). The suspension was filtered through a pad of Celite®, and the filter cake was washed with hot EtOAc (250 mL). The filtrate was concentrated under reduced pressure and partitioned between CH₂Cl₂ (25 mL) and saturated aq. NaHCO₃ (15 mL). The organic layer was separated and extracted with 1 M HCl (3×20 mL). The combined aqueous extracts were made basic with 6 M NaOH and extracted with CH₂Cl₂ (3×50 mL). The combined organic extracts were dried (Na₂SO₄) and concentrated under reduced pressure. The crude residue was purified via flash chromatography (SiO₂) eluting with CH₂Cl₂/MeOH/Et₃N (98:1:1) affording 1.27 g (79%) of MDW-2-124 as a pale yellow oil. ¹H NMR (400 MHz, CDCl₃) δ 7.50 (d, J=2.1 Hz, 1H), 7.23 (dd, J=8.2, 2.2 Hz, 1H), 6.93 (d, J=8.2 Hz, 1H), 3.60 (t, J=4.9 Hz, 1H), 2.78-2.59 (comp, 2H), 2.48 (s, 3H), 1.96-1.66 (comp, 4H).

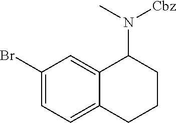 [SMILES: CN(C(=O)OCc1ccccc1)C1CCCc2ccc(Br)cc21]

Benzyl (7-bromo-1,2,3,4-tetrahydronaphthalen-1-yl)(methyl)carbamate (MDW-2-132). i-Pr₂NEt (1.36 g, 1.84 mL, 10.6 mmol) and CbzCl (1.1 g, 0.90 mL, 6.3 mmol) were added with stirring to a solution of amine MDW-2-124 (1.267 g, 5.27 mmol) in CH₂Cl₂ (21 mL) cooled to 0° C. The solution was stirred at 0° C. for 1 h and then diluted with CH₂Cl₂ (20 mL). The solution was washed with 1 N HCl (2×10 mL), 1 N NaOH (2×20 mL), saturated aqueous NaHCO₃ (1×20 mL), dried (Na₂SO₄), and concentrated under reduced pressure. The crude residue was purified via flash chromatography (SiO₂) eluting with hexanes/EtOAc (9:1) affording 1.954 g (98%) of MDW-2-132 as a clear oil. ¹H NMR (400 MHz, CDCl₃) mixture of rotamers: δ 7.47-7.29 (comp, 5H), 7.28-7.23 (comp, 2H), 6.99-6.93 (m, 1H), 5.54-5.46 and 5.37-5.30 (m, 1H), 5.29-5.19 (comp, 2H), 2.78-2.60 (comp, 5H), 2.09-1.94 (comp, 2H), 1.85-1.69 (comp, 2H). HRMS (ESI) m/z calcd for C₁₈H₂₀BrNO₂ (M+Na)⁺, 396.0570; found 396.0578.

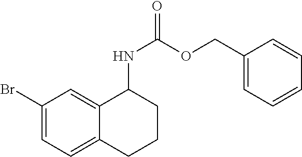 [SMILES: O=C(NC1CCCc2ccc(Br)cc21)OCc1ccccc1]

Benzyl (7-bromo-1,2,3,4-tetrahydronaphthalen-1-yl)carbamate (MDW-1-193). A solution containing 7-bromotetralone (0.500 g, 2.22 mmol), NH₄OAc (1.71 g, 22.2 mmol), and NaBH₃CN (691 mg, 11.1 mmol) in MeOH (11 mL) was stirred at 60° C. for 25 h. The reaction mixture was concentrated under reduced pressure and partitioned between CH₂Cl₂ (25 mL) and saturated aq. NaHCO₃ (25 mL). After the organic layer was separated, the aqueous layer was diluted with 6 M NaOH (5 mL) and extracted with CH₂Cl₂ (3×25 mL). The combined organic extracts were dried (Na₂SO₄), and concentrated under reduced pressure. The crude residue was taken up in CH₂Cl₂ (22 mL) and cooled to 0° C., followed by sequential addition of DIPEA (0.55 g, 0.75 mL, 4.3 mmol) and CbzCl (0.44 g, 0.37 mL, 2.6 mmol). The solution was stirred for 16 h, allowing to warm to room temperature. The reaction mixture was diluted with CH₂Cl₂ (20 mL), washed with 1 N HCl (2×25 mL), 1 N NaOH (2×25 mL), brine (1×25 mL), dried (Na₂SO₄) and concentrated under reduced pressure. The crude residue was purified via flash chromatography (SiO₂) eluting with hexanes/EtOAc (9:1) to give 0.502 g (63%) of MDW-1-193 as a white solid. ¹H NMR (400 MHz, CDCl₃): δ 7.48 (brs, J=2.1 Hz, 1H), 7.41-7.28 (comp, 5H), 7.25 (dd, J=8.4, 2.1 Hz, 1H), 6.92 (d, J=8.2 Hz, 1H), 5.40 (d, J=9.0 Hz, 1H), 5.11 (s, 2H), 4.89-4.79 (m, 1H), 2.76-2.60 (comp, 2H), 2.07-1.95 (m, 1H), 1.89-1.68 (comp, 3H). HRMS (ESI) m/z calcd for C₁₈H₁₈BrNO₂ (M+Na)⁺, 382.0413; found 382.0417.

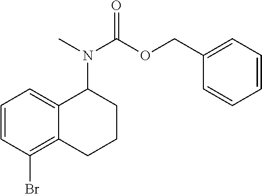 [SMILES: CN(C(=O)OCc1ccccc1)C1CCCc2c(Br)cccc21]

Benzyl (5-bromo-1,2,3,4-tetrahydronaphthalen-1-yl)(methyl)carbamate (MDW-1-192). In a modification of a literature procedure, 5-bromotetralone (0.500 g, 2.22 mmol) was dissolved in EtOH (15 mL) in a resealable tube, whereupon Ti(OiPr)₄ (3.2 g, 3.3 mL, 11.1 mmol), Et₃N (1.1 g, 1.5 mL, 10.8 mmol) and MeNH₃Cl (0.743 g, 12.5 mmol) were sequentially added.² The tube was sealed, and the reaction was stirred at room temperature for 25 h, then heated to 40° C. and stirred for 5 h. The solution was cooled to 0° C., and NaBH₄ (0.125 g, 3.30 mmol) was added in one portion. Stirring was continued at 0° C. for 1 h then stirred overnight, allowing to warm to room temperature. The mixture was added to 2 M aq. NH₄OH (20 mL). The resulting suspension was filtered through a pad of Celite®, and the filter cake was washed with hot EtOAc (250 mL). The filtrate was concentrated under reduced pressure, diluted with Et₂O (30 mL), and extracted with 1 M HCl (3×30 mL). The combined aqueous extracts were made basic with 6 M NaOH and extracted with CH₂Cl₂ (3×50 mL). The combined organic extracts were dried (Na₂SO₄) and concentrated under reduced pressure. The crude residue was taken up in CH₂Cl₂ (18 mL) and cooled to 0° C., followed by sequential addition of DIPEA (0.46 g, 0.62 mL, 3.6 mmol) and CbzCl (0.37 g, 0.31 mL, 2.2 mmol). The solution was stirred for 16 h, allowing to warm to room temperature. The reaction mixture was diluted with CH₂Cl₂ (10 mL), washed with 1 N HCl (2×25 mL), 1 N NaOH (2×25 mL), brine (1×25 mL), dried (Na₂SO₄) and concentrated under reduced pressure. The crude residue was purified via flash chromatography (SiO₂) eluting with hexanes/EtOAc (9:1) to give 0.441 g (53%) of MDW-1-192 as a light brown oil. ¹H NMR (400 MHz, CDCl₃) rotamers: δ 7.49-7.28 (comp, 6H), 7.09 (t, J=7.5 Hz, 1H), 7.05-6.99 (m, 1H), 5.60-5.53 and 5.41-5.33 (m, 1H), 5.30-5.18 (comp, 2H), 2.96-2.85 (m, 1H), 2.75-2.55 (comp, 4H), 2.10-1.95 (comp, 2H), 1.89-1.70 (comp, 2H). HRMS (ESI) m/z calcd for C₁₉H₂₀BrNO₂ (M+Na)⁺, 396.0570; found 396.0575.

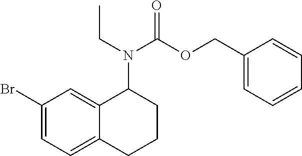 [SMILES: CCN(C(=O)OCc1ccccc1)C1CCCc2ccc(Br)cc21]

Benzyl (7-bromo-1,2,3,4-tetrahydronaphthalen-1-yl)(ethyl)carbamate (MDW-1-182). Et₃N (2.25 g, 3.1 mL, 22.2 mmol) was added to a solution containing 5-bromotetralone (500 mg, 2.22 mmol), ethylamine HCl (1.81 g, 22.2 mmol), and NaBH₃CN (279 mg, 4.44 mmol) in DCE (14.8 mL). The solution was stirred for 48 h at room temperature and then diluted with Et₂O (20 mL) and extracted with 1 N HCl (3×30 mL). The combined aqueous extracts were made basic with 6 N NaOH and extracted with CH₂Cl₂ (3×100 mL). The combined organic extracts were dried (Na₂SO₄), and concentrated under reduced pressure. The crude residue was taken up in CH₂Cl₂ (14 mL) and cooled to 0° C., followed by sequential addition of DIPEA (0.364 g, 0.490 mL, 2.84 mmol) and CbzCl (0.29 g, 0.24 mL, 1.7 mmol). The solution was stirred for 1 h, allowing to warm to room temperature. The reaction mixture was diluted with CH₂Cl₂ (20 mL), washed with 1 N HCl (2×25 mL), 1 N NaOH (2×25 mL), brine (1×25 mL), dried (Na₂SO₄) and concentrated under reduced pressure. The crude residue was purified via flash chromatography (SiO₂) eluting with hexanes/EtOAc (92:8) to give 0.432 g (50%) of MDW-1-182 as a pale yellow oil. ¹H NMR (400 MHz, CDCl₃) rotamers: δ 7.52-7.15 (comp, 7H), 7.01-6.87 (m, 1H), 5.50-5.01 (comp, 3H), 3.45-3.19 (m, 1H), 3.08-2.57 (comp, 3H), 2.17-1.92 (comp, 2H), 1.90-1.64 (comp, 2H), 1.28-1.07 (m, 3H). HRMS (ESI) m/z calcd for C₂₀H₂₂BrNO₂ (M+Na)⁺, 410.0726; found 410.0736.

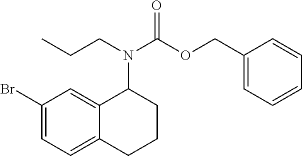 [SMILES: CCCN(C(=O)OCc1ccccc1)C1CCCc2ccc(Br)cc21]

Benzyl (7-bromo-1,2,3,4-tetrahydronaphthalen-1-yl)(propyl)carbamate (MDW-1-183). A solution of 5-bromotetralone (0.500 g, 2.22 mmol), propylamine (0.40 g, 0.55 mL 6.7 mmol), Na(OAc)BH₃ (0.942 g, 4.44 mmol), and acetic acid (0.24 g, 0.23 mL, 4.0 mmol) in DCE (22 mL) was stirred for 48 h. The reaction was diluted with Et₂O (20 mL), and extracted with 1 N HCl (3×30 mL). The combined aqueous extracts were made basic with 6 N NaOH and extracted with CH₂Cl₂ (3×100 mL). The combined organic extracts were dried (Na₂SO₄), and concentrated under reduced pressure. The crude residue was taken up in CH₂Cl₂ (10 mL) and cooled to 0° C., followed by sequential addition of DIPEA (0.27 g, 0.36 mL, 2.8 mmol) and CbzCl (0.22 g, 0.18 mL, 1.7 mmol). The solution was stirred for 1 h, allowing to warm to room temperature. The reaction mixture was diluted with CH₂Cl₂ (20 mL), washed with 1 N HCl (2×25 mL), 1 N NaOH (2×25 mL), brine (1×25 mL), dried (Na₂SO₄) and concentrated under reduced pressure. The crude residue was purified via flash chromatography (SiO₂) eluting with hexanes/EtOAc (92:8) to give 0.398 g (45%) of MDW-1-183 as a pale yellow oil. ¹H NMR (400 MHz, CDCl₃) mixture of rotamers: δ 7.46-7.14 (comp, 7H), 6.98-6.89 (m, 1H), 5.41-4.96 (comp, 3H), 3.33-3.14 (m, 1H), 2.99-2.60 (comp, 3H), 2.14-1.93 (comp, 2H), 1.92-1.65 (comp, 3H), 1.64-1.47 (m, 1H), 0.94-0.75 (comp, 3H). HRMS (ESI) m/z calcd for C₂₁H₂₄BrNO₂ (M+Na)⁺, 424.0883; found 424.0891.

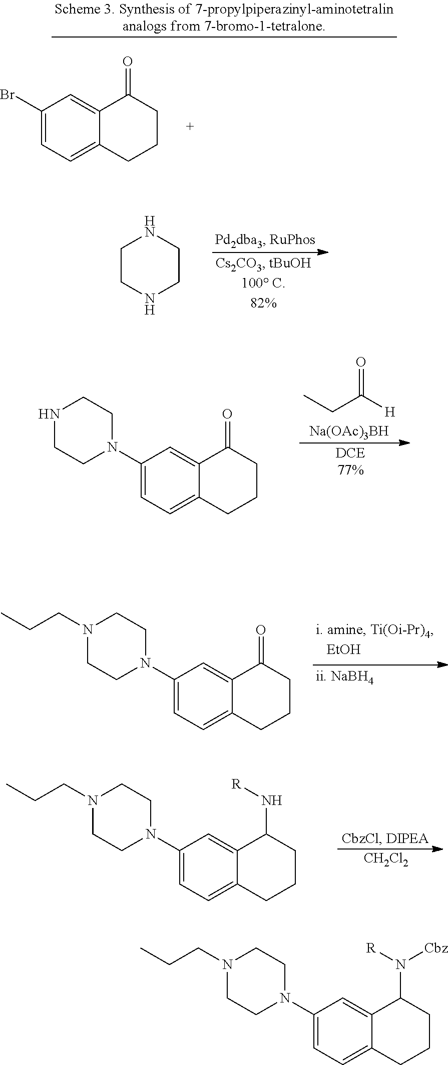

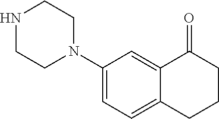 [SMILES: O=C1CCCc2ccc(N3CCNCC3)cc21]

7-(piperazin-1-yl)-3,4-dihydronaphthalen-1(2H)-one (MDW-1-262). A resealable tube was charged with 7-bromo-1-tetralone (0.500 g, 2.22 mmol), piperazine (1.913 g, 22.2 mmol), Cs₂CO₃ (1.086 g, 3.33 mmol) and degassed tBuOH (11.1 mL). The suspension was stirred at 45° C. for 15 min, whereupon a freshly prepared tBuOH solution (0.67 mL) containing Pd₂dba₃ (40.6 mg, 0.044 mmol) and RuPhos (41.5 mg, 0.088 mmol) that had been stirred at 60° C. for 30 min was added. The tube was sealed, and the reaction was stirred at 100° C. for 3 h. After cooling to room temperature, the mixture was filtered through Celite®, the filter cake was washed with CH₂Cl₂ (200 mL), and the filtrate was concentrated. The residue was dissolved in CH₂Cl₂ (50 mL), washed with saturated aq. NaHCO₃ (2×50 mL), and extracted with 1 N HCl (4×30 mL). The combined acidic aqueous extracts were made basic and extracted with CH₂C₂ (4×50 mL), after which the combined organic extracts were dried (Na₂SO₄) and concentrated under reduced pressure. The crude material was purified via flash chromatography (SiO₂) eluting with CH₂Cl₂/MeOH/Et₃N (97:2:1) affording 0.418 g (82%) of MDW-1-262 as a red oil. ¹H NMR (400 MHz, CDCl₃) δ 7.46 (d, J=2.8 Hz, 1H), 7.08 (d, J=8.4 Hz, 1H), 7.02 (dd, J=8.4, 2.8 Hz, 1H), 3.14-3.06 (comp, 4H), 3.00-2.93 (comp, 4H), 2.79 (t, J=6.1 Hz, 2H), 2.70 (br s, 1H), 2.54 (m, 2H), 2.01 (m, 2H). HRMS (ESI) m/z calcd for C₁₄H₁₈N₂O (M+H)⁺, 231.1492; found 231.1497.

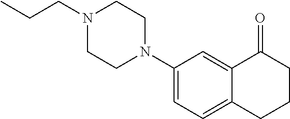 [SMILES: CCCN1CCN(c2ccc3c(c2)C(=O)CCC3)CC1]

7-(4-Propylpiperazin-1-yl)-3,4-dihydronaphthalen-1(2H)-one (MDW-2-57). A solution of propionaldehyde (0.652 g, 0.81 mL, 11.29 mmol) in DCE (25 mL) was added dropwise to a solution of amine MDW-1-234 (2.363 g, 10.3 mmol) and Na(OAc)₃BH (4.349 g, 20.5 mmol) in DCE (103 mL), and the reaction was stirred at room temperature for 3 h. The reaction mixture was then washed with saturated aq. NaHCO₃ (2×50 mL), dried (Na₂SO₄), and concentrated under reduced pressure. The crude material was purified via flash chromatography (SiO₂) eluting with hexanes/EtOAc/Et₃N (74:25:1) affording 2.165 g (77%) of MDW-2-57 as a yellow oil. ¹H NMR (400 MHz, CDCl₃) δ 7.54 (d, J=2.7 Hz, 1H), 7.14 (d, J=8.4 Hz, 1H), 7.08 (dd, J=8.5, 2.7 Hz, 1H), 3.24-3.19 (comp, 4H), 2.86 (t, J=6.1 Hz, 2H), 2.64-2.55 (comp, 6H), 2.38-2.31 (m, 2H), 2.09 (m, 2H), 1.60-1.48 (m, 2H), 0.92 (t, J=7.4 Hz, 3H). HRMS (ESI) m/z calcd for C₁₇H₂₄N₂O (M+H)⁺, 273.1961; found 273.1965.

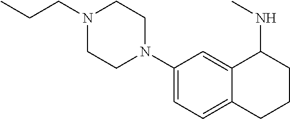 [SMILES: CCCN1CCN(c2ccc3c(c2)C(NC)CCC3)CC1]

N-methyl-7-(4-propylpiperazin-1-yl)-1,2,3,4-tetrahydronaphthalen-1-amine (MDW-2-74). In a modification of a literature procedure, tetralone MDW-2-57 (0.102 g, 0.375 mmol) was dissolved in EtOH (2.5 mL) in a resealable tube, whereupon Ti(OiPr)₄ (1.44 g, 1.5 mL, 3.75 mmol), Et₃N (0.19 g, 0.26 mL, 1.9 mmol) and MeNH₃Cl (0.127 g, 1.88 mmol) were sequentially added.² The tube was sealed, and the reaction was stirred at room temperature for 7 h. The solution was cooled to 0° C., and NaBH₄ (0.028 g, 0.75 mmol) was added in one portion. Stirring was continued at 0° C. for 1 h, and the mixture was added to 2 M aq. NH₄OH (10 mL). The suspension was filtered through a pad of Celite®, and the filter cake was washed with hot EtOAc (150 mL). The filtrate was concentrated under reduced pressure and partitioned between CH₂Cl₂ (15 mL) and saturated aq. NaHCO₃ (10 mL). The organic layer was separated and extracted with 1 M HCl (3×15 mL). The combined aqueous extracts were adjusted to pH 10 with 6 M NaOH and extracted with CH₂Cl₂ (3×50 mL). The combined organic extracts were dried (Na₂SO₄) and concentrated under reduced pressure. The crude residue was purified via flash chromatography (SiO₂) eluting with CH₂Cl₂/MeOH/Et₃N (98:1:1) affording 0.0763 g (78%) of MDW-2-74 as a pale yellow oil. ¹H NMR (400 MHz, CDCl₃) δ 6.97 (d, J=8.4 Hz, 1H), 6.92 (d, J=2.6 Hz, 1H), 6.77 (dd, J=8.4, 2.7 Hz, 1H), 3.61 (t, J=4.9 Hz, 1H), 3.20-3.14 (comp, 4H), 2.77-2.62 (comp, 2H), 2.62-2.57 (comp, 4H), 2.49 (s, 3H), 2.38-2.32 (comp, 2H), 1.96-1.81 (comp, 3H), 1.75-1.66 (m, 1H), 1.60-1.49 (comp, 2H), 1.37 (br s, 1H), 0.92 (t, J=7.4 Hz, 3H). HRMS (ESI) m/z calcd for C₁₈H₂₉N₃(M+H)⁺, 288.2434; found 288.2438.

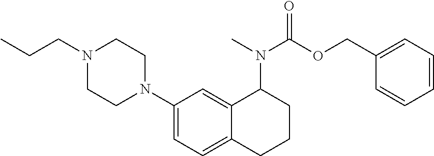 [SMILES: CCCN1CCN(c2ccc3c(c2)C(N(C)C(=O)OCc2ccccc2)CCC3)CC1]

Benzyl methyl(7-(4-propylpiperazin-1-yl)-1,2,3,4-tetrahydronaphthalen-1-yl)carbamate (MDW-2-76). i-Pr₂NEt (0.0519 g, 70 μL, 0.377 mmol) and CbzCl (0.0478 g, 40 L, 0.276 mmol) were added with stirring to a solution of amine MDW-2-74 (0.0721 g, 0.251 mmol) in CH₂Cl₂ (1.3 mL) cooled to 0° C. The solution was stirred at 0° C. for 4 h and then diluted with CH₂Cl₂ (10 mL). The solution was washed with 1 N HCl (2×10 mL), 1 N NaOH (2×10 mL), saturated aqueous NaHCO₃ (1×10 mL), dried (Na₂SO₄), and concentrated under reduced pressure. The crude residue was purified via flash chromatography (SiO₂) eluting with hexanes/EtOAc/Et₃N (74:25:1) affording 0.0969 g (92%) of (MDW-2-76) as a clear oil. ¹H NMR (499 MHz, CDCl₃) rotamers δ 7.45-7.27 (comp, 5H), 7.02-6.96 (m, 1H), 6.81-6.75 (m, 1H), 6.66-6.60 (m, 1H), 5.51-5.11 (comp, 3H), 3.16-3.05 (comp, 4H), 2.75-2.61 (comp, 5H), 2.61-2.54 (comp, 4H), 2.39-2.33 (comp, 2H), 2.08-1.92 (comp, 2H), 1.83-1.68 (comp, 2H), 1.61-1.51 (comp, 2H), 0.94 (t, J=7.4 Hz, 3H). HRMS (ESI) m/z calcd for C₂₆H₃₅N₃O₂ (M+H)⁺, 422.2802; found 422.2816.

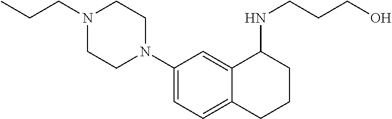 [SMILES: CCCN1CCN(c2ccc3c(c2)C(NCCCO)CCC3)CC1]

3-((7-(4-Propylpiperazin-1-yl)-1,2,3,4-tetrahydronaphthalen-1-yl)amino)propan-1-ol (MDW-2-140). Ti(OiPr)₄ (0.73 g, 0.76 mL, 0.0026 mmol) was added to a solution of tetralone MDW-2-57 (0.0698 g, 0.256 mmol) and 3-amino-1-propanol (0.028 g, 0.029 mL, 0.38 mmol) in EtOH (1.7 mL) in a screw cap vial. The vial was sealed and the reaction was stirred at 45° C. for 20 h. The solution was cooled to 0° C., and NaBH₄ (0.019 g, 0.50 mmol) was added in one portion. Stirring was continued at 0° C. for 1 h, and the mixture was added to 2 M aq. NH₄OH (10 mL). The suspension was filtered through a pad of Celite®, and the filter cake was washed with hot EtOAc (150 mL). The filtrate was concentrated under reduced pressure and partitioned between CH₂C₂ (15 mL) and 1 N NaOH (10 mL). The organic layer was separated and extracted with 1 M HCl (3×15 mL). The combined aqueous extracts were made basic with 6 M NaOH and extracted with CH₂C₂ (3×50 mL). The combined organic extracts were dried (Na₂SO₄) and concentrated under reduced pressure. The crude residue was purified via flash chromatography (SiO₂) eluting with CH₂Cl₂/MeOH/Et₃N (97:2:1) affording 0.0772 g (91%) of MDW-2-140 as an orange oil. ¹H NMR (499 MHz, CDCl₃) δ 6.95 (d, J=8.4 Hz, 1H), 6.91 (d, J=2.6 Hz, 1H), 6.77 (dd, J=8.4, 2.6 Hz, 1H), 4.37 (br s, 2H), 3.83-3.75 (comp, 3H), 3.20-3.10 (comp, 4H), 3.04-2.90 (comp, 2H), 2.72-2.54 (comp, 6H), 2.37-2.28 (comp, 2H), 1.93-1.65 (comp, 6H), 1.57-1.47 (comp, 2H), 0.90 (t, J=7.4 Hz, 3H). HRMS (ESI) m/z calcd for C₂₀H₃₃N₃O (M+H)⁺, 332.2696; found 332.27110.

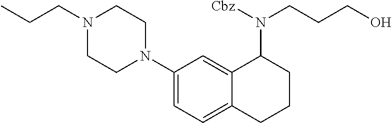 [SMILES: CCCN1CCN(c2ccc3c(c2)C(N(CCCO)C(=O)OCc2ccccc2)CCC3)CC1]

Benzyl (3-hydroxypropyl)(7-(4-propylpiperazin-1-yl)-1,2,3,4-tetrahydronaphthalen-1-yl)carbamate (MDW-2-143). i-Pr₂NEt (0.060 g, 0.081 mL, 0.47 mmol) and CbzCl (0.043 g, 0.036 mL, 0.25 mmol) were added with stirring to a solution of amine MDW-2-74 (0.0770 g, 0.232 mmol) in CH₂Cl₂ (2.3 mL) cooled to 0° C. The solution was stirred at 0° C. for 1 h and then allowed to warm to room temperature and stirred for 16 h. The solution was diluted with MeOH (3 mL) followed by addition of 1 N NaOH (3 mL) and the mixture was stirred for 1 h. The volatile organic solvents were removed under reduced pressure and the solution was diluted with CH₂Cl₂ (20 mL), then washed with 1 N NaOH (2×20 mL), dried (Na₂SO₄), and concentrated under reduced pressure. The crude residue was purified via flash chromatography (SiO₂) eluting with EtOAc/hexanes/MeOH/Et₃N (50:44:5:1) affording 0.0203 g (19%) of (MDW-2-142) as a clear oil. ¹H NMR (499 MHz, CDCl₃) δ 7.46-7.15 (comp, 5H), 6.98 (d, J=8.3 Hz, 1H), 6.75 (dd, J=8.4, 2.5 Hz, 1H), 6.63-6.52 (m, 1H), 5.43-5.03 (comp, 3H), 3.66-3.41 (comp, 3H), 3.27-3.06 (comp, 5H), 2.99-2.60 (comp, 7H), 2.59-2.46 (comp, 2H), 2.08-1.60 (comp, 8H), 0.95 (t, J=7.3 Hz, 3H). HRMS (ESI) m/z calcd for C₂₈H₃₉N₃O₃ (M+Na)⁺, 488.2884; found 488.2895.

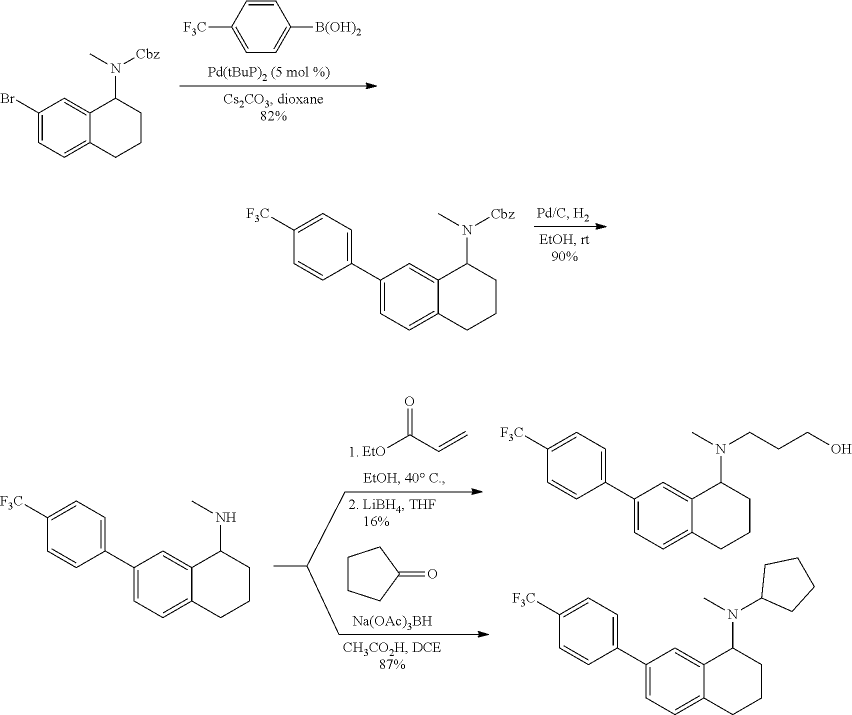

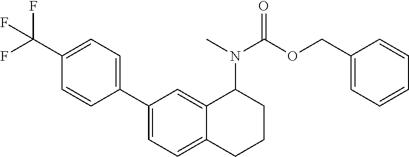 [SMILES: CN(C(=O)OCc1ccccc1)C1CCCc2ccc(-c3ccc(C(F)(F)F)cc3)cc21]

Benzyl methyl(7-(4-(trifluoromethyl)phenyl)-1,2,3,4-tetrahydronaphthalen-1-yl)carbamate (MDW-2-133). A resealable tube was charged with carbamate MDW-2-132 (0.220 g, 0.667 mmol), 4-trifluoromethylphenylboronic acid (0.254 g, 1.34 mmol), cesium carbonate (0.435 g, 1.34 mmol), and Pd(t-Bu₃P)₂ (0.17 g, 0.033 mmol). Degassed 1,4-dioxane (1.7 mL) was added to the tube and the reaction was stirred at 100° C. for 6 h. The solution was then diluted with CH₂Cl₂ (20 mL) and partitioned between 1 N NaOH (20 mL). The organic layer was separated and the aqueous layer was extracted with CH₂C₂ (2×20 mL). The combined organic extracts were dried (Na₂SO₄), concentrated under reduced pressure, and the crude residue was purified via flash chromatography (SiO₂) eluting with hexanes/EtOAc (9:1) affording 0.242 g (82%) of (MDW-2-133) as a pale yellow oil. ¹H NMR (400 MHz, CDCl₃)) δ 7.76-7.57 (comp, 4H), 7.52-7.31 (comp, 7H), 7.25 (d, J=8.0 Hz, 1H), 5.72-5.19 (comp, 3H), 2.96-2.71 (comp, 5H), 2.22-2.03 (comp, 2H), 1.99-1.80 (comp, 2H). HRMS (ESI) m/z calcd for C₂₆H₂₄F₃NO₂(M+H)⁺, 440.1832; found 440.1849.

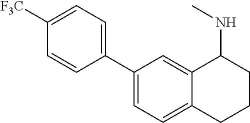 [SMILES: CNC1CCCc2ccc(-c3ccc(C(F)(F)F)cc3)cc21]

N-methyl-7-(4-(trifluoromethyl)phenyl)-1,2,3,4-tetrahydronaphthalen-1-amine (MDW-1-147). H₂ gas was bubbled through a solution containing carbamate MDW-2-133 (0.0706 g, 0.166 mmol) and 10 wt. % Pd/C (0.0265 g) in EtOH (4.2 mL) for 5 min. The solution was stirred under a hydrogen atmosphere (1 atm) for 4 h, filtered through Celite® washing the filter cake with CH₂Cl₂ (100 mL), and concentrated under reduced pressure to afford MDW-1-147 that was used without further purification. ¹H NMR (400 MHz, CDCl₃) δ 7.81 (d, J=1.9 Hz, 1H), 7.76 (d, J=8.1 Hz, 2H), 7.64 (d, J=8.1 Hz, 2H), 7.42 (dd, J=8.1, 1.9 Hz, 1H), 7.17 (d, J=8.0 Hz, 1H), 5.94 (br s, 1H), 4.04-3.98 (m, 1H), 2.94-2.70 (comp, 2H), 2.51 (s, 3H), 2.11-1.97 (comp, 3H), 1.83-1.71 (m, 1H).

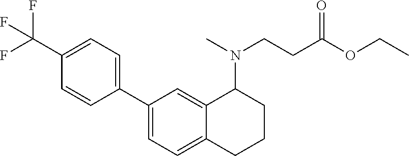 [SMILES: CCOC(=O)CCN(C)C1CCCc2ccc(-c3ccc(C(F)(F)F)cc3)cc21]

Ethyl 3-(methyl(7-(4-(trifluoromethyl)phenyl)-1,2,3,4-tetrahydronaphthalen-1-yl)amino)propanoate (MDW-1-143): Prepared from amine MDW-1-142 (0.071 g, 0.16 mmol) according to the general procedure for conjugate addition to ethyl acrylate. The crude reside was purified via flash chromatography (SiO₂) eluting with 1% Et₃N/hexanes to afford 0.016 g (26%) of MDW-1-143 as a pale yellow oil. HRMS (ESI) m/z calcd for C₂₅H₃₃N₃O₂ (M+Na)⁺, 430.2645; found 430.2483.

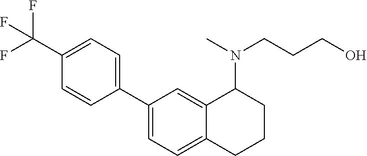 [SMILES: CN(CCCO)C1CCCc2ccc(-c3ccc(C(F)(F)F)cc3)cc21]

3-(Methyl(7-(4-(trifluoromethyl)phenyl)-1,2,3,4-tetrahydronaphthalen-1-yl)amino)propan-1-ol (MDW-1-144). LiBH₄ was added to a solution of ester MDW-1-143 in THF (1 mL) and the reaction was stirred for 24 h at room temperature. The reaction was then quenched with MeOH (1 mL) and concentrated under reduced pressure. The residue was dissolved in CH₂C₂ (5 mL) washed with water (1×5 mL), brine (1×5 mL), dried (Na₂SO₄), and concentrated under reduced pressure. The crude residue was purified via flash chromatography (SiO₂) eluting with hexanes/EtOAc/Et₃N (74:25:1) affording 9 mg (63%) of MDW-1-144 as a clear oil. ¹H NMR NMR (499 MHz, CDCl₃): δ 7.81 (brs, 1H), 7.74 (d, J=8.2 Hz, 2H), 7.67 (d, J=8.2 Hz, 2H), 7.41 (dd, J=7.9, 2.1 Hz, 1H), 7.18 (d, J=7.9 Hz, 1H), 4.09-4.02 (m, 1H), 3.86-3.80 (m, 1H), 3.78-3.73 (m, 1H), 2.82-2.72 (m, 4H), 2.32 (s, 3H), 2.05-1.98 (m, 2H), 1.83-1.65 (m, 5H).

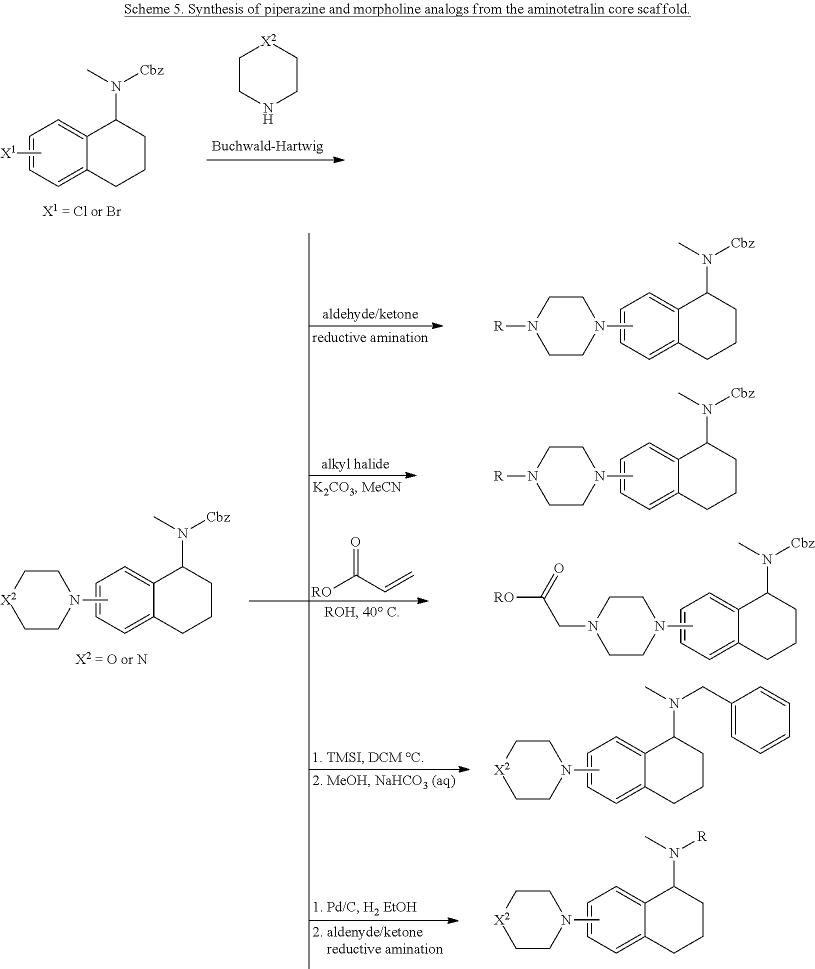

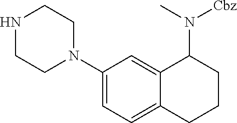 [SMILES: CN(C(=O)OCc1ccccc1)C1CCCc2ccc(N3CCNCC3)cc21]

Benzyl methyl(7-(piperazin-1-yl)-1,2,3,4-tetrahydronaphthalen-1-yl)carbamate (MDW-2-111). A resealable tube was charged with carbamate MDW-2-132 (1.610 g, 4.257 mmol), piperazine (1.833 g, 21.28 mmol), NaOt-Bu (0.614 g, 6.39 mmol) and degassed THF (23.6 mL). The suspension was stirred at 45° C. for 15 min, whereupon a freshly prepared THF solution (3.9 mL) containing Pd₂dba₃ (0.078 g, 0.085 mmol) and RuPhos (0.079 g, 0.17 mmol) that had been stirred at 45° C. for 30 min was added. The tube was sealed, and the reaction was stirred at 65° C. for 17 h. After cooling to room temperature, the mixture was filtered through Celite®, the filter cake was washed with CH₂C₂ (200 mL), and the filtrate was concentrated. The residue was dissolved in CH₂C₂ (50 mL), washed with saturated aq. NaHCO₃ (2×50 mL), and extracted with 1 N HCl (4×30 mL). The combined acidic aqueous extracts were made basic and extracted with CH₂Cl₂ (4×50 mL), after which the combined organic extracts were dried (Na₂SO₄) and concentrated under reduced pressure. The crude material was purified via flash chromatography (SiO₂) eluting with CH₂Cl₂/MeOH/Et₃N (97:2:1) affording 1.222 g (76%) of MDW-2-111 as an orange oil. ¹H NMR (400 MHz, CDCl₃) (rotamers) δ 7.45-7.28 (comp, 5H), 6.99 (d, J=8.3 Hz, 1H), 6.77-6.71 (m, 1H), 6.65-6.59 (m, 1H), 5.51-5.11 (comp, 3 H), 3.05-2.97 (comp, 8H), 2.74-2.61 (comp, 5H), 2.09-1.91 (comp, 2H), 1.85-1.68 (comp, 2H), 1.59 (br s, 1H). HRMS (ESI) m/z calcd for C₂₄H₃₁N₃O₂ (M+H)⁺, 394.2489; found 394.2495.

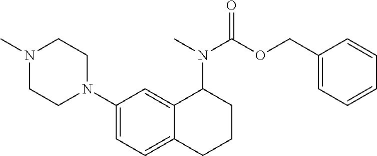 [SMILES: CN1CCN(c2ccc3c(c2)C(N(C)C(=O)OCc2ccccc2)CCC3)CC1]

Benzyl methyl(7-(4-methylpiperazin-1-yl)-1,2,3,4-tetrahydronaphthalen-1-yl)carbamate (MDW-1-93). Prepared from carbamate MDW-1-158 (40 mg, 0.121 mmol) and the 1-methylpiperazine (26.9 mg, 0.242 mmol) according to the representative procedure for Buchwald-Hartwig cross coupling. The crude residue was purified via flash chromatography (SiO₂) eluting with hexanes/EtOAc/Et₃N (69:30:1) to afford 0.010 g (15%) of MDW-1-93 as a pale yellow oil. ¹H NMR (400 MHz, CDCl₃) (rotamers)¹H NMR (400 MHz, CDCl₃) δ 7.46-7.27 (comp, 5H), 7.03-6.96 (m, 1H), 6.81-6.72 (m, 1H), 6.66-6.58 (m, 1H), 5.50-5.10 (comp, 3H), 3.21-3.06 (comp, 4H), 2.73-2.58 (comp, 9H), 2.45-2.38 (comp, 3H), 2.09-1.90 (comp, 2H), 1.84-1.66 (comp, 2H). HRMS (ESI) m/z calcd for C₂₄H₃₁N₃O₂ (M+H)⁺, 394.2489; found 394.2495.

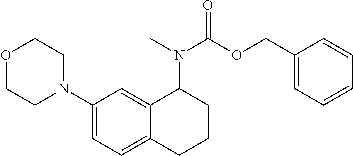 [SMILES: CN(C(=O)OCc1ccccc1)C1CCCc2ccc(N3CCOCC3)cc21]

Benzyl methyl(7-morpholino-1,2,3,4-tetrahydronaphthalen-1-yl)carbamate (MDW-1-126). Prepared from carbamate MDW-1-158 (0.1100 g, 0.303 mmol) and morpholine (0.080 g, 0.080 mL, 0.91 mmol) according to the representative procedure for Buchwald-Hartwig cross coupling. The crude residue was purified via flash chromatography (SiO₂) eluting with hexanes/EtOAc/Et₃N (84:15:1) affording 0.0742 g (64%) of MDW-1-126 as a pale yellow oil. ¹H NMR (400 MHz, CDCl₃) δ 7.46-7.28 (comp, 5H), 7.01 (d, J=8.4 Hz, 1H), 6.78-6.73 (m, 1H), 6.63-6.55 (m, 1H), 5.52-5.10 (comp, 3H), 3.86-3.79 (comp, 4H), 3.06-2.97 (comp, 4H), 2.73-2.63 (comp, 5H), 2.10-1.92 (comp, 2H), 1.83-1.67 (comp, 2H). HRMS (ESI) m/z calcd for C₂₃H₂₆N₂O₃ (M+Na)⁺, 403.1992; found 403.1997.

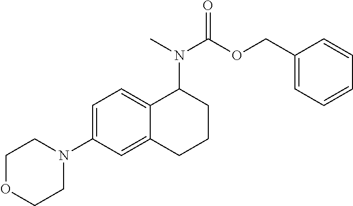 [SMILES: CN(C(=O)OCc1ccccc1)C1CCCc2cc(N3CCOCC3)ccc21]

Benzyl methyl(7-morpholino-1,2,3,4-tetrahydronaphthalen-1-yl)carbamate (MDW-1-124). Prepared from carbamate MDW-1-121 (0.100 g, 0.303 mmol) and morpholine (0.080 g, 0.080 mL, 0.91 mmol) according to the representative procedure for Buchwald-Hartwig cross coupling. The crude residue was purified via flash chromatography (SiO₂) eluting with hexanes/EtOAc/Et₃N (79:20:1) to afford 0.0676 g (59%) of MDW-1-124 as a pale yellow oil. ¹H NMR (400 MHz, CDCl₃) rotamers δ 7.45-7.28 (comp, 5H), 7.03-6.96 (m, 1H), 6.77-6.71 (m, 1H), 6.63-6.60 (m, 1H), 5.49-5.10 (comp, 3H), 3.88-3.82 (comp, 4H), 3.18-3.08 (comp, 4H), 2.83-2.59 (comp, 5H), 2.08-1.92 (comp, 2H), 1.85-1.68 (comp, 2H). HRMS (ESI) m/z calcd for C₂₃H₂₆N₂O₃ (M+Na)⁺, 403.1992; found 403.2000.

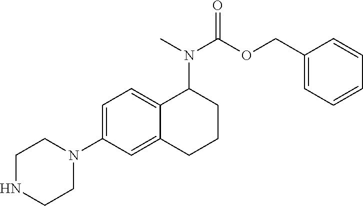 [SMILES: CN(C(=O)OCc1ccccc1)C1CCCc2cc(N3CCNCC3)ccc21]

Benzyl methyl(6-(piperazin-1-yl)-1,2,3,4-tetrahydronaphthalen-1-yl)carbamate (MDW-1-165). Prepared from carbamate MDW-1-158 (0.300 g, 0.910 mmol) and piperazine according to the representative procedure for Buchwald-Hartwig cross coupling. The crude residue was purified via flash chromatography (SiO₂) eluting with DCM/MeOH/Et₃N (97:2:1) to afford 0.114 g (35%) of MDW-1-165 as a pale yellow oil. ¹H NMR (400 MHz, CDCl₃) mixture of rotamers: δ 7.44-7.23 (comp, 5H), 7.00-6.92 (m, 1H), 6.77-6.69 (m, 1H), 6.60 (brs, 1H), 5.46-5.40 and 5.34-5.27 (m, 1H), 5.25-5.13 (comp, 2H), 3.15-3.05 (comp, 4H), 3.00 (comp, 4H), 2.72-2.59 (comp, 6H), 2.04-1.88 (comp, 2H), 1.82-1.63 (comp, 2H). HRMS (ESI) m/z calcd for C₂₃H₂₉N₃O₂ (M+H)⁺, 380.2333; found 380.2346.

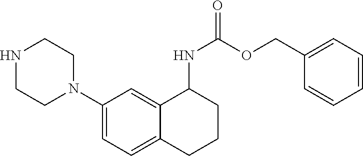 [SMILES: O=C(NC1CCCc2ccc(N3CCNCC3)cc21)OCc1ccccc1]

Benzyl (7-(piperazin-1-yl)-1,2,3,4-tetrahydronaphthalen-1-yl)carbamate (MDW-1-197). Prepared from carbamate MDW-1-193 (0.200 g, 0.555 mmol) and piperazine according to the representative procedure for Buchwald-Hartwig cross coupling. The crude reside was purified via flash chromatography (SiO₂) eluting with DCM/MeOH/Et₃N (97:2:1) to give 0.0888 g (43%) of MDW-1-197 as a white solid. ¹H NMR (400 MHz, CDCl₃) δ 7.42-7.28 (comp, 5H), 6.96 (d, J=8.4 Hz, 1H), 6.84 (d, J=2.6 Hz, 1H), 6.76 (dd, J=8.4, 2.6 Hz, 1H), 5.33 (d, J=8.7 Hz, 1H), 5.19-5.08 (comp, 2H), 4.89-4.81 (m, 1H), 3.07-2.93 (comp, 8H), 2.71-2.64 (comp, 2H), 2.61-2.49 (m, 1H), 2.04-1.94 (m, 1H), 1.87-1.73 (comp, 3H).

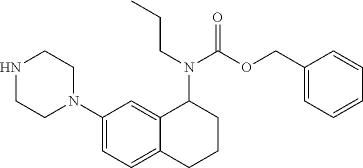 [SMILES: CCCN(C(=O)OCc1ccccc1)C1CCCc2ccc(N3CCNCC3)cc21]

Benzyl (7-(piperazin-1-yl)-1,2,3,4-tetrahydronaphthalen-1-yl)(propyl)carbamate (MDW-1-186). Prepared from carbamate MDW-1-183 (0.398 g, 1.02 mmol) and piperazine according to the representative procedure for Buchwald-Hartwig cross coupling. The crude residue was purified via flash chromatography (SiO₂) eluting with DCM/MeOH/Et₃N (97:2:1) to afford 0.284 g (70%) of MDW-1-186 as a yellow oil. ¹H NMR (400 MHz, CDCl₃) mixture of rotamers: δ 7.43-7.18 (comp, 5H), 6.95 (d, J=8.4 Hz, 1H), 6.73 (dd, J=8.4, 2.6 Hz, 1H), 6.58 (m, 1H), 5.42-4.99 (comp, 3H), 3.26-3.04 (m, 1H), 3.02-2.87 (comp, 8H), 2.83-2.59 (comp, 3H), 2.10-1.97 (comp, 2H), 1.97-1.88 (m, 1H), 1.84-1.60 (comp, 3H), 1.59-1.44 (m, 1H), 0.81 and 0.73 (t, J=7.4 Hz, 3H). HRMS (ESI) m/z calcd for C₂₅H₃₃N₃O₂ (M+H)⁺, 408.2646; found 408.2661.

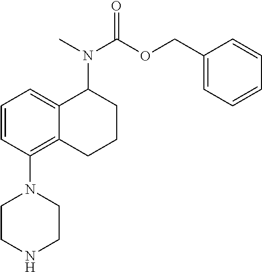 [SMILES: CN(C(=O)OCc1ccccc1)C1CCCc2c1cccc2N1CCNCC1]

Benzyl methyl(5-(piperazin-1-yl)-1,2,3,4-tetrahydronaphthalen-1-yl)carbamate (MDW-1-212). Prepared from carbamate MDW-1-192 (0.129 g, 0.339 mmol) and piperazine according to the representative procedure for Buchwald-Hartwig cross coupling. The crude residue was purified via flash chromatography (SiO₂) eluting with DCM/MeOH/Et₃N (97:2:1) to afford 0.0735 g (57%) of MDW-1-212 as a pale yellow oil. ¹H NMR (400 MHz, CDCl₃) mixture of rotamers: δ 7.45-7.27 (comp, 5H), 7.13 (t, J=7.8 Hz, 1H), 6.95-6.84 (comp, 2H), 5.56-5.49 and 5.42-5.35 (m, 1H), 5.28-5.14 (comp, 2H), 3.07-2.92 (comp, 6H), 2.81-2.72 (comp, 2H), 2.72-2.59 (comp, 5H), 2.55-2.42 (m, 1H), 2.10-1.93 (comp, 2H), 1.86-1.61 (comp, 2H). HRMS (ESI) m/z calcd for C₂₃H₂₉N₃O₂ (M+H)⁺, 380.2333; found 380.2348.

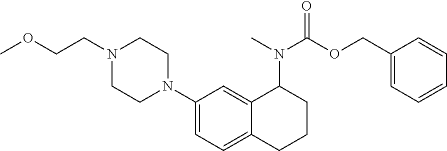 [SMILES: COCCN1CCN(c2ccc3c(c2)C(N(C)C(=O)OCc2ccccc2)CCC3)CC1]

Benzyl (7-(4-(2-methoxyethyl)piperazin-1-yl)-1,2,3,4-tetrahydronaphthalen-1-yl)(methyl)carbamate (MDW-1-219). Prepared from carbamate MDW-2-132 (0.070 g, 0.19 mmol) and N-(2-Methoxyethyl)piperazine (0.041, 0.042 mL, 0.28 mmol) according to the representative procedure for Buchwald-Hartwig cross-coupling. The crude residue was purified via flash chromatography (SiO₂) eluting with hexanes/EtOAc/Et₃N (59:40:1) to give 0.040 g (49%) of MDW-1-219 as a pale yellow oil. ¹H NMR (400 MHz, CDCl₃) rotamers: δ 7.45-7.27 (comp, 5H), 6.98 (d, J=8.4 Hz, 1H), 6.79-6.74 (m, 1H), 6.65-6.58 (m, 1H), 5.50-5.43 and 5.38-5.32 (m, 1H), 5.31-5.11 (comp, 2H), 3.56 (t, J=5.5 Hz, 2H), 3.38 (s, 3H), 3.17-3.06 (comp, 4H), 2.76-2.59 (comp, 11H), 2.07-1.91 (comp, 2H), 1.83-1.68 (comp, 2H). HRMS (ESI) m/z calcd for C₂₆H₃₅N₃O₃ (M+H)⁺, 438.2751; found 438.2767.

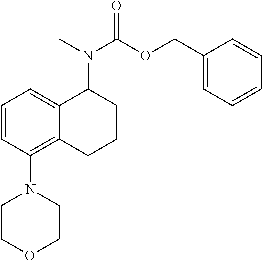 [SMILES: CN(C(=O)OCc1ccccc1)C1CCCc2c1cccc2N1CCOCC1]

Benzyl methyl(5-morpholino-1,2,3,4-tetrahydronaphthalen-1-yl)carbamate (MDW-1-199). Prepared from carbamate MDW-1-192 (0.100 g, 0.267 mmol) and morpholine (0.030 g, 0.030 mL, 0.35 mmol) according to the representative procedure for Buchwald-Hartwig cross-coupling. The crude residue was purified via flash chromatography (SiO₂) eluting with hexanes/EtOAc/Et₃N (76:23:1) to give 0.0526 g (52%) of MDW-1-199 as a pale yellow oil. ¹H NMR (499 MHz, CDCl₃) rotamers: δ 7.45-7.28 (m, 5H), 7.16 (t, J=7.8 Hz, 1H), 6.96-6.88 (m, 2H), 5.57-5.51 and 5.43-5.37 (m, 1H), 5.28-5.16 (m, 2H), 3.91-3.78 (m, 4H), 3.05-2.95 (m, 3H), 2.83-2.74 (m, 2H), 2.67 and 2.66 (s, 3H), 2.56-2.44 (m, 1H), 2.11-1.96 (m, 2H), 1.87-1.63 (m, 2H). HRMS (ESI) m/z calcd for C₂₃H₂₈N₂O₃ (M+H)⁺, 381.2173; found 381.2180.

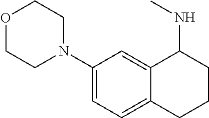 [SMILES: CNC1CCCc2ccc(N3CCOCC3)cc21]

N-methyl-7-morpholino-1,2,3,4-tetrahydronaphthalen-1-amine (MDW-1-130). H₂ gas was bubbled through a solution containing carbamate MDW-1-126 (0.0681 g, 0.179 mmol) and 10 wt. % Pd/C (0.029 g) in EtOH (4.5 mL) for 5 min. The solution was stirred under a hydrogen atmosphere (1 atm) for 1 h, filtered through Celite® washing the filter cake with CH₂Cl₂ (100 mL), and concentrated under reduced pressure. The combined filtrate and washings was concentrated under reduced pressure to afford 0.0423 g of MDW-1-130, which was used without further purification.

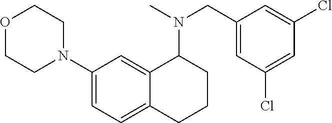 [SMILES: CN(Cc1cc(Cl)cc(Cl)c1)C1CCCc2ccc(N3CCOCC3)cc21]

N-(3,5-dichlorobenzyl)-N-methyl-7-morpholino-1,2,3,4-tetrahydronaphthalen-1-amine (MDW-1-131). Prepared from amine MDW-1-130 (0.020 g, 0.081 mmol) and 3,5-dichlorobenzaldehyde (0.028 g, 0.16 mmol) according to the representative procedure for reductive amination with an aldehyde. The crude residue was purified via flash chromatography (SiO₂) eluting with Hexanes/EtOAc/Et₃N (96:3:1) to afford 0.020 g (62%) of MDW-1-131 as a clear oil. ¹H NMR (400 MHz, CDCl₃) δ 7.44 (d, J=2.5 Hz, 1H), 7.30 (d, J=1.9 Hz, 2H), 7.21 (t, J=1.9 Hz, 1H), 6.98 (d, J=8.3 Hz, 1H), 6.75 (dd, J=8.3, 2.6 Hz, 1H), 3.93-3.84 (comp, 5H), 3.60 (d, J=14.3 Hz, 1H), 3.35 (d, J=14.2 Hz, 1H), 3.20-3.08 (m, 4H), 2.75-2.62 (comp, 2H), 2.30 (s, 3H), 2.11-1.96 (comp, 2H), 1.70-1.61 (comp, 2H). HRMS (ESI) m/z calcd for C₂₂H₂₆Cl₂N₂O (M+H)⁺, 405.1495; found 405.1498.

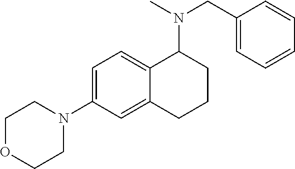 [SMILES: CN(Cc1ccccc1)C1CCCc2cc(N3CCOCC3)ccc21]

N-Benzyl-N-methyl-6-morpholino-1,2,3,4-tetrahydronaphthalen-1-amine (MDW-1-129). TMSI (0.062 g, 0.044 mL, 0.31 mmol) was added to a solution of carbamate MDW-1-124 (0.0599 g, 0.155 mmol) in CH₂Cl₂ (2.6 mL) that was cooled to 0° C. in a foil wrapped flask. The reaction was stirred at 0° C. in the absence of light for 3 h followed by sequential addition of MeOH (2 mL) and saturated aq. NaHCO₃ (2 mL). The reaction was stirred for 17 h followed by removal of MeOH under reduced pressure. The reaction mixture was diluted with Et₂O (15 mL) and extracted with 3 N HCl (4×15 mL). The combined aqueous extracts were made basic with 6 N NaOH and extracted with CH₂Cl₂ (3×30 mL). The combined CH₂Cl₂ extract was washed with brine (1×30 mL), dried (Na₂SO₄), and concentrated under reduced pressure. The crude residue was purified via flash chromatography (SiO₂) eluting with hexanes/EtOAc/Et₃N (97:3:1 to 85:15:1 gradient) afforded 0.0251 g (47%) of MDW-1-129 as a pale yellow oil. ¹H NMR (499 MHz, CDCl₃) δ 7.75 (d, J=8.6 Hz, 1H), 7.40 (d, J=7.1 Hz, 2H), 7.32 (t, J=7.5 Hz, 2H), 7.25-7.21 (m, 1H), 6.82 (dd, J=8.6, 2.7 Hz, 1H), 6.60 (d, J=2.7 Hz, 1H), 3.95-3.89 (m, 1H), 3.86 (t, J=4.8 Hz, 4H), 3.68 (d, J=13.4 Hz, 1H), 3.51 (d, J=13.4 Hz, 1H), 3.18-3.10 (comp, 4H), 2.81-2.64 (comp, 2H), 2.19 (s, 3H), 2.06-1.96 (comp, 2H), 1.76-1.62 (comp, 2H). LRMS (ESI) m/z calcd for C₂₂H₂₈N₂O (M+H)⁺, 337.2; found 337.2.

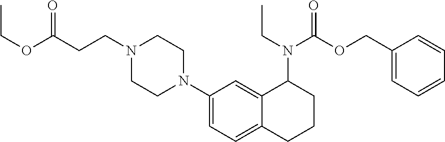 [SMILES: CCOC(=O)CCN1CCN(c2ccc3c(c2)C(N(CC)C(=O)OCc2ccccc2)CCC3)CC1]

Ethyl 3-(4-(8-(((benzyloxy)carbonyl)(ethyl)amino)-5,6,7,8-tetrahydronaphthalen-2-yl)piperazin-1-yl)propanoate (MDW-1-190). Prepared from amine MDW-1-185 (0.030 g, 0.076 mmol) according to the general procedure for conjugate addition to ethyl acrylate. The crude residue was purified via flash chromatography (SiO₂) eluting with hexanes/EtOAc/Et₃N (69:30:1) to afford 0.035 g (92%) of MDW-1-190 as a pale yellow oil. ¹H NMR (499 MHz, CDCl₃) rotamers δ 7.45-7.23 (comp, 5H), 6.97 (d, J=8.3 Hz, 1H), 6.75 (dd, J=8.5, 2.5 Hz, 1H), 6.63-6.58 (m, 1H), 5.45-5.04 (comp, 3H), 4.16 (q, J=7.1 Hz, 2H), 3.36-3.15 (m, 1H), 3.09-3.00 (comp, 4H), 2.96-2.79 (m, 1H), 2.75 (t, J=7.4 Hz, 2H), 2.71-2.63 (comp, 2H), 2.61-2.56 (comp, 4H), 2.53 (t, J=7.4 Hz, 2H), 2.11-1.91 (comp, 2H), 1.87-1.67 (comp, 2H), 1.27 (t, J=7.1 Hz, 3H), 1.18 and 1.11 (t, J=7.0 Hz, 3H). HRMS (ESI) m/z calcd for C₂₉H₃₉N₃O₄ (M+H)⁺, 494.3013; found 494.3034.

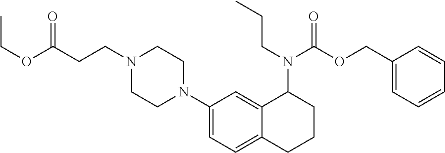 [SMILES: CCCN(C(=O)OCc1ccccc1)C1CCCc2ccc(N3CCN(CCC(=O)OCC)CC3)cc21]

Ethyl 3-(4-(8-(((benzyloxy)carbonyl)(propyl)amino)-5,6,7,8-tetrahydronaphthalen-2-yl)piperazin-1-yl)propanoate (MDW-1-191). Prepared from amine MDW-1-186 (0.030 g, 0.074 mmol) according to the general procedure for conjugate addition to ethyl acrylate. The crude residue was purified via flash chromatography (SiO₂) eluting with hexanes/EtOAc/Et₃N (69:30:1) to afford 0.032 g (86%) of MDW-1-191 as a pale yellow oil. ¹H NMR (499 MHz, CDCl₃) rotamers δ 7.43-7.19 (comp, 5H), 7.00-6.94 (m, 1H), 6.75 (dd, J=8.4, 2.5 Hz, 1H), 6.61-6.56 (m, 1H), 5.42-5.02 (comp, 3H), 4.16 (q, J=7.1 Hz, 2H), 3.25-2.98 (comp, 5H), 2.78-2.63 (comp, 5H), 2.53 (t, J=7.4 Hz, 2H), 2.09-1.92 (comp, 2H), 1.89-1.61 (comp, 3H), 1.59-1.45 (m, 1H), 1.27 (t, J=7.1 Hz, 3H), 0.82 and 0.74 (t, J=7.4 Hz, 3H). HRMS (ESI) m/z calcd for C₃₀H₄₁N₃O₄ (M+H)⁺, 508.3170; found 508.3191.

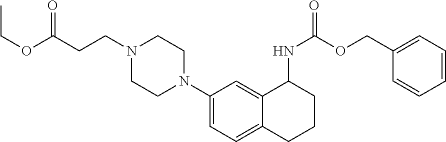 [SMILES: CCOC(=O)CCN1CCN(c2ccc3c(c2)C(NC(=O)OCc2ccccc2)CCC3)CC1]

Ethyl 3-(4-(8-(((benzyloxy)carbonyl)amino)-5,6,7,8-tetrahydronaphthalen-2-yl)piperazin-1-yl)propanoate (MDW-1-206). Prepared from amine MDW-1-197 (0.030 g, 0.082 mmol) according to the general procedure for conjugate addition to ethyl acrylate. The cruse residue was purified via flash chromatography (SiO₂) eluting with hexanes/EtOAc/Et₃N (69:30:1) to afford 0.031 g (80%) of MDW-1-197 as a pale yellow oil. ¹H NMR (400 MHz, CDCl₃) δ 7.43-7.29 (comp, 5H), 6.98 (d, J=8.4 Hz, 1H), 6.86 (d, J=2.6 Hz, 1H), 6.78 (dd, J=8.4, 2.6 Hz, 1H), 5.20-5.10 (comp, 2H), 5.02 (d, J=8.7 Hz, 1H), 4.90-4.83 (m, 1H), 4.16 (q, J=7.1 Hz, 2H), 3.17-3.05 (comp, 4H), 2.75 (t, J=7.4 Hz, 2H), 2.72-2.63 (comp, 2H), 2.60 (t, J=5.0 Hz, 4H), 2.53 (t, J=7.4 Hz, 2H), 2.07-1.96 (m, 1H), 1.88-1.74 (comp, 3H), 1.27 (t, J=7.1 Hz, 3H). HRMS (ESI) m/z calcd for C₂₇H₃₅N₃O₄ (M+H)⁺, 466.2700; found 466.2721.

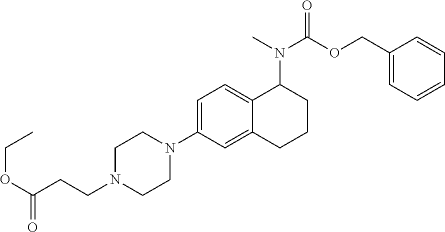 [SMILES: CCOC(=O)CCN1CCN(c2ccc3c(c2)CCCC3N(C)C(=O)OCc2ccccc2)CC1]

Ethyl 3-(4-(5-(((benzyloxy)carbonyl)(methyl)amino)-5,6,7,8-tetrahydronaphthalen-2-yl)piperazin-1-yl)propanoate (MDW-1-207). Prepared from amine MDW-1-165 (0.030 g, 0.079 mmol) according to the general procedure for conjugate addition to ethyl acrylate. The crude residue was purified via flash chromatography (SiO₂) eluting with hexanes/EtOAc/Et₃N (69:30:1) to afford 0.032 g (84%) of MDW-1-207 as a pale yellow oil. ¹H NMR (499 MHz, CDCl₃) rotamers δ 7.44-7.25 (comp, 5H), 7.01-6.94 (m, 1H), 6.77-6.70 (m, 1H), 6.63-6.59 (m, 1H), 5.48-5.28 (m, 1H), 5.27-5.15 (comp, 2H), 4.15 (q, J=7.1 Hz, 2H), 3.21-3.11 (comp, 4H), 2.79-2.66 (comp, 4H), 2.66-2.58 (comp, 7H), 2.53 (t, J=7.4 Hz, 2H), 2.07-1.90 (comp, 2H), 1.83-1.65 (comp, 2H), 1.26 (t, J=7.1 Hz, 3H). HRMS (ESI) m/z calcd for C₂₈H₃₇N₃O₄ (M+H)⁺, 480.2857; found 480.2876.

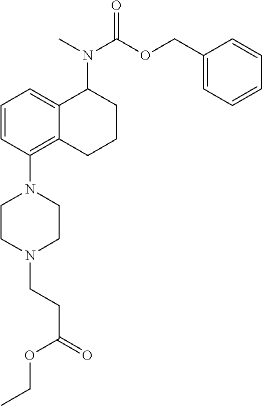

Ethyl 3-(4-(5-(((benzyloxy)carbonyl)(methyl)amino)-5,6,7,8-tetrahydronaphthalen-1-yl)piperazin-1-yl)propanoate (MDW-1-214). Prepared from amine MDW-1-212 (0.030 g, 0.079 mmol) and ethyl acrylate according to the general procedure for conjugate addition to ethyl acrylate. The crude residue was purified via flash chromatography (SiO₂) eluting with hexanes/EtOAc/Et₃N (69:30:1) to afford 0.032 g (59%) of MDW-1-214 as a pale yellow oil. ¹H NMR (499 MHz, CDCl₃) rotamers δ 7.43-7.27 (comp, 5H), 7.13 (t, J=7.8 Hz, 1H), 6.94-6.90 (m, 1H), 6.90-6.85 (m, 1H), 5.55-5.35 (m, 1H), 5.26-5.11 (comp, 2H), 4.16 (q, J=7.2 Hz, 2H), 3.04-2.92 (comp, 3H), 2.85-2.75 (comp, 4H), 2.71-2.42 (comp, 10H), 2.10-1.93 (comp, 2H), 1.85-1.62 (comp, 2H), 1.27 (t, J=7.1 Hz, 3H). HRMS (ESI) m/z calcd for C₂₈H₃₇N₃O₄ (M+H)⁺, 480.2857; found 480.2875.

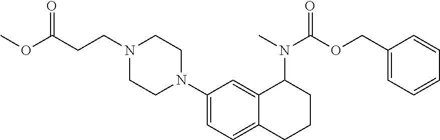 [SMILES: COC(=O)CCN1CCN(c2ccc3c(c2)C(N(C)C(=O)OCc2ccccc2)CCC3)CC1]

Methyl 3-(4-(8-(((benzyloxy)carbonyl)(methyl)amino)-5,6,7,8-tetrahydronaphthalen-2-yl)piperazin-1-yl)propanoate (MDW-1-166). A solution of amine MDW-2-111 (0.060 g, 0.16 mmol) and methyl acrylate (0.16 g, 0.17 mL, 1.6 mmol) in MeOH (0.8 mL) was stirred at 40° C. for 18 h. The reaction was concentrated under reduce pressure and the crude residue was purified via flash chromatography (SiO₂) eluting with hexanes/EtOAc/Et₃N (59:40:1) to afford 0.056 g (75%) of MDW-1-166 as a pale yellow oil. ¹H NMR (499 MHz, CDCl₃) rotamers δ 7.46-7.26 (comp, 5H), 6.99 (d, J=8.4 Hz, 1H), 6.79-6.73 (m, 1H), 6.64-6.57 (m, 1H), 5.50-5.32 (m, 1H), 5.31-5.12 (comp, 2H), 3.72-3.68 (comp, 3H), 3.11-3.02 (comp, 4H), 2.76 (t, J=7.4 Hz, 2H), 2.71-2.62 (comp, 5H), 2.62-2.53 (comp, 6H), 2.06-1.92 (comp, 2H), 1.83-1.68 (comp, 2H). HRMS (ESI) m/z calcd for C₂₇H₃₅N₃O₄ (M+Na)⁺, 488.2520; found 488.2541.

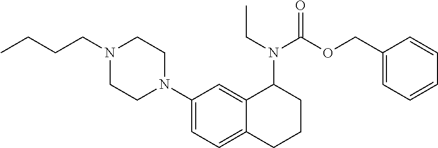 [SMILES: CCCCN1CCN(c2ccc3c(c2)C(N(CC)C(=O)OCc2ccccc2)CCC3)CC1]

Benzyl ethyl(7-(4-propylpiperazin-1-yl)-1,2,3,4-tetrahydronaphthalen-1-yl)carbamate (MDW-1-195). Prepared from amine MDW-1-185 (0.029 g 0.072 mmol) and propionaldehyde according to the representative procedure for reductive amination with an aldehyde. The crude residue was purified via flash chromatography (SiO₂) eluting with hexanes/EtOAc/Et₃N (76:23:1) to afford 0.024 g (77%) of MDW-1-195 as a pale yellow oil. ¹H NMR (499 MHz, CDCl₃) rotamers δ 7.45-7.22 (comp, 5H), 6.98 (d, J=8.4 Hz, 1H), 6.77 (dd, J=8.4, 2.5 Hz, 1H), 6.62 (br s, 1H), 5.47-5.04 (comp, 3H), 3.35-3.16 (m, 1H), 3.11-3.02 (comp, 4H), 2.98-2.79 (m, 1H), 2.77-2.62 (comp, 2H), 2.60-2.52 (comp, 4H), 2.39-2.32 (comp, 2H), 2.11-2.01 (m, 1H), 2.00-1.91 (m, 1H), 1.88-1.66 (comp, 2H), 1.60-1.50 (comp, 2H), 1.18 and 1.12 (t, J=7.0 Hz, 3H), 0.94 (t, J=7.4 Hz, 3H). HRMS (ESI) m/z calcd for C₂₇H₃₇N₃O₂ (M+H)⁺, 436.2959; found 436.2976.

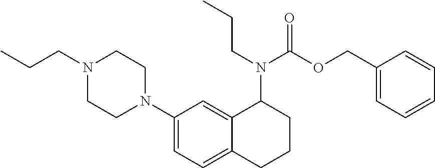 [SMILES: CCCN1CCN(c2ccc3c(c2)C(N(CCC)C(=O)OCc2ccccc2)CCC3)CC1]

Benzyl propyl(7-(4-propylpiperazin-1-yl)-1,2,3,4-tetrahydronaphthalen-1-yl)carbamate (MDW-1-196). Prepared from amine MDW-1-186 (0.029 g 0.071 mmol) and propionaldehyde according to the representative procedure for reductive amination with an aldehyde. The crude residue was purified via flash chromatography (SiO₂) eluting with hexanes/EtOAc/Et₃N (76:23:1) to afford 0.023 g (72%) of MDW-1-196 as a pale yellow oil. ¹H NMR (400 MHz, CDCl₃) rotamers δ 7.47-7.17 (comp, 5H), 6.98 (d, J=8.5 Hz, 1H), 6.77 (dd, J=8.4, 2.5 Hz, 1H), 6.60 (d, J=2.6 Hz, 1H), 5.44-5.01 (comp, 3H), 3.27-2.99 (comp, 5H), 2.86-2.61 (comp, 3H), 2.60-2.51 (comp, 4H), 2.40-2.31 (comp, 2H), 2.11-2.00 (m, 1H), 2.00-1.91 (m, 1H), 1.91-1.62 (comp, 3H), 1.61-1.45 (comp, 3H), 0.94 (t, J=7.4 Hz, 3H), 0.82 and 0.75 (t, J=7.4 Hz, 1H). HRMS (ESI) m/z calcd for C₂₈H₃₉N₃O₂ (M+H)⁺, 450.3115; found 450.3134.

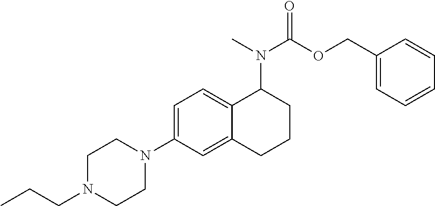 [SMILES: CCCN1CCN(c2ccc3c(c2)CCCC3N(C)C(=O)OCc2ccccc2)CC1]

Benzyl methyl(6-(4-propylpiperazin-1-yl)-1,2,3,4-tetrahydronaphthalen-1-yl)carbamate (MDW-1-204). Prepared from amine MDW-1-165 (0.030 g 0.079 mmol) and propionaldehyde according to the general procedure for reductive amination with an aldehyde. The crude residue was purified via flash chromatography (SiO₂) eluting with hexanes/EtOAc/Et₃N (76:23:1) to afford 0.024 g (71%) of MDW-1-204 as a pale yellow oil. ¹H NMR (400 MHz, CDCl₃) δ 7.45-7.26 (comp, 5H), 6.97 (dd, J=8.6, 5.1 Hz, 1H), 6.79-6.71 (m, 1H), 6.65-6.59 (m, 1H), 5.49-5.28 (m, 1H), 5.27-5.11 (comp, 2H), 3.25-3.13 (comp, 4H), 2.84-2.56 (comp, 9H), 2.41-2.32 (comp, 2H), 2.08-1.90 (comp, 2H), 1.86-1.64 (comp, 2H), 1.63-1.48 (comp, 2H), 0.93 (t, J=7.4 Hz, 3H). HRMS (ESI) m/z calcd for C₂₆H₃₅N₃O₂ (M+H)⁺, 422.2802; found 422.2819.

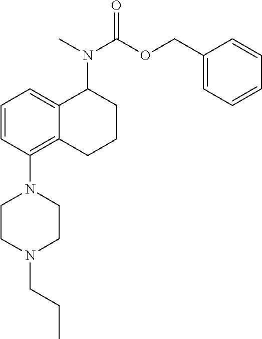

Benzyl methyl(5-(4-propylpiperazin-1-yl)-1,2,3,4-tetrahydronaphthalen-1-yl)carbamate (MDW-1-215). Prepared from amine MDW-1-212 (0.030 g 0.079 mmol) and propionaldehyde according to the representative procedure for reductive amination with an aldehyde. The crude residue was purified via flash chromatography (SiO₂) eluting with hexanes/EtOAc/Et₃N (82:17:1) to afford 0.023 g (68%) of MDW-1-215 as a pale yellow oil. ¹H NMR (499 MHz, CDCl₃) rotamers δ 7.43-7.28 (comp, 5H), 7.13 (t, J=7.8 Hz, 1H), 6.96-6.92 (m, 1H), 6.90-6.85 (m, 1H), 5.56-5.35 (m, 1H), 5.26-5.11 (comp, 2H), 3.07-2.93 (comp, 3H), 2.87-2.78 (comp, 2H), 2.70-2.42 (comp, 8H), 2.41-2.35 (comp, 2H), 2.10-1.94 (comp, 2H), 1.85-1.64 (comp, 2H), 1.61-1.51 (comp, 2H), 0.94 (t, J=7.4 Hz, 3H).

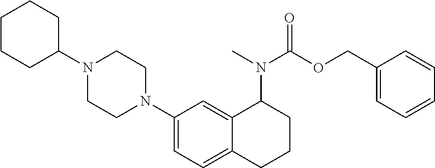 [SMILES: CN(C(=O)OCc1ccccc1)C1CCCc2ccc(N3CCN(C4CCCCC4)CC3)cc21]

Benzyl (7-(4-cyclohexylpiperazin-1-yl)-1,2,3,4-tetrahydronaphthalen-1-yl)(methyl)carbamate (MDW-1-205). Prepared from amine MDW-2-111 (0.030 g 0.079 mmol) and cyclohexanone according to the representative procedure for reductive amination with a ketone. The crude residue was purified via flash chromatography (SiO₂) eluting with hexanes/EtOAc/Et₃N (74:25:1) to afford 0.025 g (69%) of MDW-1-205 as a pale yellow oil. ¹H NMR (499 MHz, CDCl₃) rotamers δ 7.46-7.27 (comp, 5H), 6.98 (d, J=8.4 Hz, 1H), 6.81-6.75 (m, 1H), 6.66-6.59 (m, 1H), 5.50-5.32 (m, 1H), 5.31-5.10 (comp, 2H), 3.15-3.03 (comp, 4H), 2.76-2.60 (comp, 9H), 2.34-2.25 (m, 1H), 2.07-1.89 (comp, 4H), 1.85-1.69 (comp, 4H), 1.68-1.61 (m, 1H), 1.30-1.21 (comp, 4H), 1.18-1.09 (m, 1H). HRMS (ESI) m/z calcd for C₂₉H₃₉N₃O₂ (M+Na)⁺, 484.2934; found 484.2942.

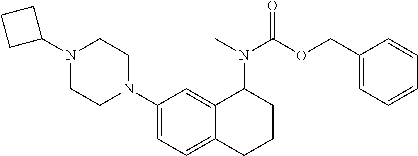 [SMILES: CN(C(=O)OCc1ccccc1)C1CCCc2ccc(N3CCN(C4CCC4)CC3)cc21]

Benzyl (7-(4-cyclobutylpiperazin-1-yl)-1,2,3,4-tetrahydronaphthalen-1-yl)(methyl)carbamate (MDW-1-162). Prepared from carbamate MDW-2-111 (0.025 g 0.066 mmol) and cyclobutanone according to the representative procedure for reductive amination with a ketone. The crude residue was purified via flash chromatography (SiO₂) eluting with hexanes/EtOAc/Et₃N (74:25:1) to afford 0.025 g (89%) of MDW-1-162 as a pale yellow oil. ¹H NMR (499 MHz, CDCl₃) rotamers δ 7.43-7.28 (comp, 5H), 6.98 (d, J=8.2 Hz, 1H), 6.80-6.74 (m, 1H), 6.62 (dd, J=8.3, 2.2 Hz, 1H), 5.50-5.31 (m, 1H), 5.30-5.12 (comp, 2H), 3.15-3.05 (comp, 4H), 2.84-2.61 (comp, 6H), 2.51-2.42 (comp, 4H), 2.11-1.89 (comp, 6H), 1.78-1.68 (comp, 4H). HRMS (ESI) m/z calcd for C₂₇H₃₅N₃O₂ (M+H)⁺, 434.2802; found 434.2819.

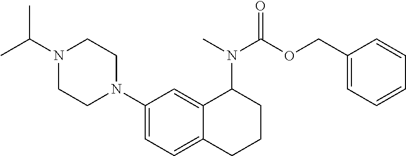 [SMILES: CC(C)N1CCN(c2ccc3c(c2)C(N(C)C(=O)OCc2ccccc2)CCC3)CC1]

Benzyl (7-(4-isopropylpiperazin-1-yl)-1,2,3,4-tetrahydronaphthalen-1-yl)(methyl)carbamate (MDW-1-167). Prepared from carbamate MDW-2-111 (0.025 g 0.066 mmol) and acetone according to the representative procedure for reductive amination with a ketone. The crude residue was purified via flash chromatography (SiO₂) eluting with hexanes/EtOAc/Et₃N (64:35:1) to afford 0.025 g (89%) of MDW-1-167 as a pale yellow oil. ¹H NMR (499 MHz, CDCl₃) rotamers δ 7.45-7.28 (comp, 5H), 6.99 (d, J=8.3 Hz, 1H), 6.80-6.75 (m, 1H), 6.63 (dd, J=10.1, 2.2 Hz, 1H), 5.50-5.32 (m, 1H), 5.30-5.11 (comp, 2H), 3.15-3.05 (comp, 4H), 2.75-2.61 (comp, 10H), 2.07-1.92 (comp, 2H), 1.83-1.70 (comp, 2H). HRMS (ESI) m/z calcd for C₂₆H₃₅N₃O₂ (M+Na)⁺, 444.2641; found 444.2630.

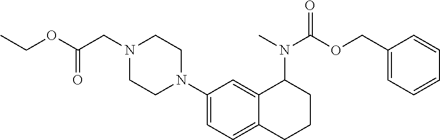 [SMILES: CCOC(=O)CN1CCN(c2ccc3c(c2)C(N(C)C(=O)OCc2ccccc2)CCC3)CC1]

Ethyl 2-(4-(8-(((benzyloxy)carbonyl)(methyl)amino)-5,6,7,8-tetrahydronaphthalen-2-yl)piperazin-1-yl)acetate (MDW-1-169). Prepared from amine MDW-2-111 (0.025 g 0.066 mmol) and ethyl bromoacetate according to the representative procedure for alkylation of amines with an alkyl halide. The crude residue was purified via flash chromatography (SiO₂) eluting with hexanes/EtOAc/Et₃N (69:30:1) to afford 0.020 mg (62%) of MDW-1-169 as a pale yellow oil. ¹H NMR (499 MHz CDCl₃) rotamers δ 7.45-7.27 (comp, 5H), 6.99 (d, J=8.4 Hz, 1H), 6.80-6.74 (m, 1H), 6.62 (dd, J=12.4, 2.0 Hz, 1H), 5.49-5.32 (m, 1H), 5.31-5.10 (comp, 2H), 4.25-4.17 (comp, 2H), 3.26 (s, 2H), 3.18-3.07 (comp, 4H), 2.74-2.62 (comp, 9H), 2.06-1.92 (comp, 2H), 1.83-1.67 (comp, 2H), 1.32-1.27 (m, 3H). HRMS (ESI) m/z calcd for C₂₇H₃₅N₃O₄ (M+H)⁺, 466.2700; found 466.2715.

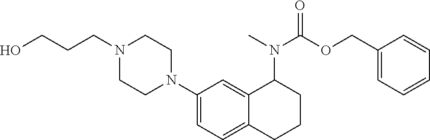 [SMILES: CN(C(=O)OCc1ccccc1)C1CCCc2ccc(N3CCN(CCCO)CC3)cc21]

Benzyl (7-(4-(3-hydroxypropyl)piperazin-1-yl)-1,2,3,4-tetrahydronaphthalen-1-yl)(methyl)carbamate (MDW-1-177). Prepared from amine MDW-2-111 (0.100 g 0.264 mmol) and 3-bromo-1-propanol according to the representative procedure for alkylation of amines with an alkyl halide. Purification via flash chromatography (SiO₂) eluting with EtOAc/Et₃N (99:1) to give 0.039 g (34%) of MDW-1-177 as a pale yellow oil. ¹H NMR (499 MHz, CDCl₃) rotamers δ 7.44-7.28 (comp, 5H), 6.98 (d, J=8.3 Hz, 1H), 6.78-6.71 (m, 1H), 6.59 (dd, J=12.4, 2.6 Hz, 1H), 5.49-5.31 (m, 1H), 5.30-5.10 (comp, 2H), 3.82 (t, J=5.2 Hz, 2H), 3.13-3.01 (comp, 4H), 2.74-2.61 (comp, 12H), 2.05-1.91 (comp, 2H), 1.82-1.68 (comp, 4H). HRMS (ESI) m/z calcd for C₂₆H₃₅N₃O₃ (M+H)⁺, 438.2751; found 438.2759.

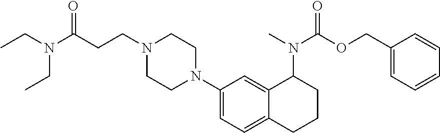 [SMILES: CCN(CC)C(=O)CCN1CCN(c2ccc3c(c2)C(N(C)C(=O)OCc2ccccc2)CCC3)CC1]

Benzyl (7-(4-(3-(diethylamino)-3-oxopropyl)piperazin-1-yl)-1,2,3,4-tetrahydronaphthalen-1-yl)(methyl)carbamate (MDW-1-213). Prepared from amine MDW-2-111 (0.030 g 0.079 mmol) and known 3-bromo-N,N-diethylpropanamide according to general procedure for alkylation.³ Purification via flash chromatography (SiO₂) eluting with EtOAc/Et₃N (99:1) to give 14 mg (36%) of MDW-1-213 as a pale yellow oil. ¹H NMR (499 MHz, CDCl₃) rotamers δ 7.45-7.28 (comp, 5H), 6.99 (d, J=8.4 Hz, 1H), 6.79-6.74 (m, 1H), 6.64-6.59 (m, 1H), 5.49-5.32 (m, 1H), 5.30-5.11 (comp, 2H), 3.42-3.30 (comp, 4H), 3.14-3.04 (comp, 4H), 2.84-2.77 (comp, 2H), 2.74-2.61 (comp, 9H), 2.59-2.53 (comp, 2H), 2.07-1.92 (comp, 2H), 1.80-1.71 (comp, 2H), 1.22-1.16 (m, 3H), 1.12 (t, J=7.1 Hz, 3H). HRMS (ESI) m/z calcd for C₃₀H₄₂N₄O₃ (M+H)⁺, 507.3330; found 507.3346.

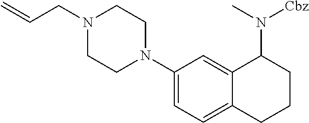 [SMILES: C=CCN1CCN(c2ccc3c(c2)C(N(C)C(=O)OCc2ccccc2)CCC3)CC1]

Benzyl (7-(4-allylpiperazin-1-yl)-1,2,3,4-tetrahydronaphthalen-1-yl)(methyl)carbamate (MDW-2-131). A solution of amine MDW-2-111 (0.0549 g, 0.145 mmol), allyl bromide (0.014 mL, 0.020 g, 0.16 mmol), and K₂CO₃ (0.040 g, 0.29 mmol) in MeCN (1.4 mL) was stirred at room temperature for 26 hours. The solution was diluted with CH₂Cl₂ (20 mL), washed with 1 N aq. NaOH (1×20 mL), dried (Na₂SO₄), and concentrated under reduced pressure. The crude residue was purified via flash chromatography (SiO₂) eluting with Et₂O/Hexanes/Et₃N (60:39:1) affording 0.0184 g (30%) of MDW-2-131 as a clear oil. ¹H NMR (499 MHz, CDCl₃) δ 7.45-7.27 (comp, 5H), 6.99 (d, J=8.3 Hz, 1H), 6.80-6.74 (m, 1H), 6.65-6.59 (m, 1H), 5.95-5.85 (m, 1H), 5.50-5.09 (comp, 5H), 3.15-3.01 (comp, 6H), 2.74-2.52 (comp, 9H), 2.08-1.91 (comp, 2H), 1.75 (comp, 2H). HRMS (ESI) m/z calcd for C₂₆H₃₃N₃O₂ (M+H)⁺, 420.2646; found 420.2659.

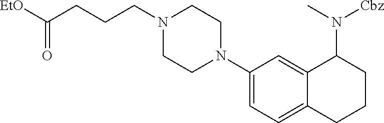 [SMILES: CCOC(=O)CCCN1CCN(c2ccc3c(c2)C(N(C)C(=O)OCc2ccccc2)CCC3)CC1]

Ethyl 4-(4-(8-(((benzyloxy)carbonyl)(methyl)amino)-5,6,7,8-tetrahydronaphthalen-2-yl)piperazin-1-yl)butanoate (MDW-2-130). A solution of amine MDW-2-111 (0.0545 g, 0.144 mmol), Ethyl 4-bromobutyrate (0.023 mL, 0.031 g, 0.16 mmol), and K₂CO₃ (0.040 g, 0.29 mmol) in MeCN (1.4 mL) was stirred at room temperature for 26 hours. The solution was diluted with CH₂C₂ (20 mL), washed with 1 N aq. NaOH (1×20 mL), dried (Na₂SO₄), and concentrated under reduced pressure. The crude residue was purified via flash chromatography (SiO₂) eluting with Et₂O/Hexanes/Et₃N (75:24:1) affording 0.0390 g (55%) of MDW-2-130 as a clear oil. ¹H NMR (499 MHz, CDCl₃) δ 7.45-7.28 (comp, 5H), 6.99 (d, J=8.3 Hz, 1H), 6.79-6.73 (m, 1H), 6.65-6.58 (m, 1H), 5.51-5.11 (comp, 3H), 4.14 (q, J=7.1 Hz, 2H), 3.13-3.02 (comp, 4H), 2.75-2.61 (comp, 5H), 2.60-2.53 (comp, 4H), 2.42 (t, J=7.3 Hz, 2H), 2.37 (t, J=7.4 Hz, 2H), 2.08-1.91 (comp, 2H), 1.90-1.67 (comp, 4H), 1.26 (t, J=7.1 Hz, 3H). HRMS (ESI) m/z calcd for C₂₉H₃₉N₃O₄ (M+H)⁺, 494.3013; found 494.3027.

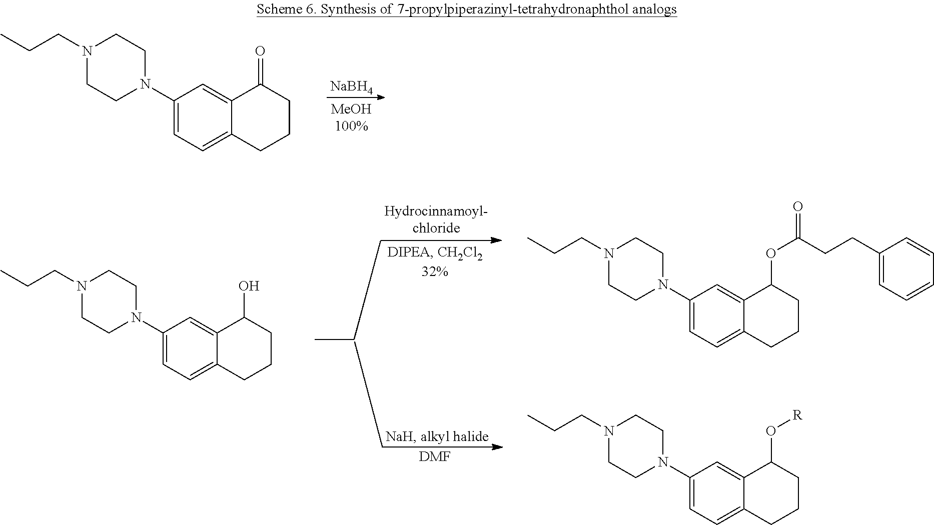

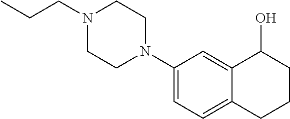 [SMILES: CCCN1CCN(c2ccc3c(c2)C(O)CCC3)CC1]

7-(4-Propylpiperazin-1-yl)-1,2,3,4-tetrahydronaphthalen-1-ol (MDW-2-65). NaBH₄ (0.062 g, 1.6 mmol) was added to a solution of ketone MDW-2-57 (0.0150 g, 0.551 mmol) in MeOH (2.7 mL) and the mixture was stirred for 2 h at room temperature. The reaction was quenched with 1 N NaOH (5 mL), the MeOH was removed under reduced pressure, and the aqueous solution was extracted with CH₂Cl₂ (3×10 mL). The combined organic extracts were dried (Na₂SO₄) and concentrated under reduced pressure to afford 0.151 g (100%) of MDW-2-65 as a clear oil that was used without further purification. ¹H NMR (400 MHz, CDCl₃) δ 6.98 (d, J=2.6 Hz, 1H), 6.94 (d, J=8.4 Hz, 1H), 6.75 (dd, J=8.4, 2.7 Hz, 1H), 4.68-4.63 (m, 1H), 3.14-3.07 (comp, 4H), 2.93 (br s, 1H), 2.72-2.50 (comp, 6H), 2.35-2.27 (comp, 2H), 2.00-1.64 (comp, 4H), 1.58-1.47 (comp, 2H), 0.90 (t, J=7.4 Hz, 3H). HRMS (ESI) m/z calcd for C₁₇H₂₆N₂O (M+H)⁺, 275.2118; found 275.2125.

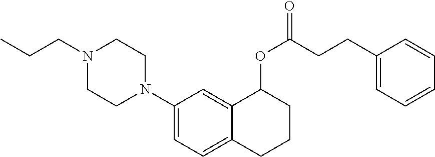 [SMILES: CCCN1CCN(c2ccc3c(c2)C(OC(=O)CCc2ccccc2)CCC3)CC1]

7-(4-Propylpiperazin-1-yl)-1,2,3,4-tetrahydronaphthalen-1-yl 3-phenylpropanoate (MDW-1-290). Hydrocinnamoyl chloride (0.018 g, 0.016 mL, 0.109 mmol) was added to a solution of alcohol MDW-2-65 (0.030 g, 0.109 mmol) and DIPEA (0.014 g, 0.019 mL, 0.109 mmol) in CH₂Cl₂ cooled to 0° C. The cooled solution was stirred for 5 min, then allowed to warm to room temperature and stirred for 19 h. The reaction mixture was diluted with CH₂Cl₂ (10 mL), washed with 1 N HCl (1×10 mL), 1 N NaOH (1×10 mL), saturated aq. NaHCO₃ (1×20 mL), dried (Na₂SO₄), and concentrated under reduced pressure. The crude residue was purified via flash chromatography (SiO₂) eluting with hexanes/EtOAc/Et₃N (79:20:1) affording 0.024 g (32%) of MDW-1-290 as a pale yellow oil. ¹H NMR (500 MHz, CDCl₃) δ 7.29-7.25 (comp, 2H), 7.22-7.17 (comp, 3H), 7.02 (d, J=8.4 Hz, 1H), 6.85 (dd, J=8.4, 2.7 Hz, 1H), 6.78 (d, J=2.6 Hz, 1H), 5.97-5.93 (m, 1H), 3.20-3.14 (comp, 4H), 2.97 (t, J=7.7 Hz, 2H), 2.80-2.60 (comp, 8H), 2.45-2.38 (comp, 2H), 1.94-1.83 (comp, 3H), 1.79-1.71 (m, 1H), 1.64-1.54 (comp, 2H), 0.94 (t, J=7.3 Hz, 3H). HRMS (ESI) m/z calcd for C₂₆H₃₄N₂O₂ (M+H)⁺, 407.2693; found 407.2697.

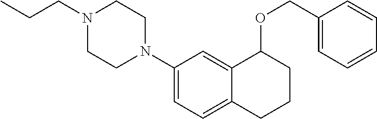 [SMILES: CCCN1CCN(c2ccc3c(c2)C(OCc2ccccc2)CCC3)CC1]

1-(8-(Benzyloxy)-5,6,7,8-tetrahydronaphthalen-2-yl)-4-propylpiperazine (MDW-1-245). A solution of alcohol MDW-2-65 (0.020 g, 0.073 mmol) and 60% NaH dispersion in mineral oil (0.0058 g, 0.146 mmol) in DMF (0.36 mL) was stirred at room temperature for 30 min and then cooled to 0° C. A solution of benzyl bromide (0.012 g, 8.6 μL, 0.073 mmol) in DMF (0.09 mL) was added and the solution was stirred at 0° C. for 30 minutes then allowed to warm to room temperature and stirred for 1 h. The reaction was quenched with water (0.5 mL), diluted with CH₂C₂ (10 mL), washed with saturated aq. NH₄Cl (1×10 mL), dried (Na₂SO₄), and concentrated under reduced pressure. The crude residue was purified via flash chromatography (SiO₂) eluting with hexanes/EtOAc/Et₃N (89:10:1) affording 0.015 g of MDW-1-245 (58%) as a pale yellow oil. ¹H NMR (600 MHz, CDCl₃) δ 7.43-7.39 (comp, 2H), 7.38-7.33 (comp, 2H), 7.30-7.26 (m, 1H), 6.99 (d, J=8.4 Hz, 1H), 6.86 (d, J=2.7 Hz, 1H), 6.81 (dd, J=8.4, 2.7 Hz, 1H), 4.70 (d, J=12.0 Hz, 1H), 4.59 (d, J=12.0 Hz, 1H), 4.51-4.47 (m, 1H), 3.21-3.15 (comp, 4H), 2.79-2.60 (comp, 6H), 2.44-2.38 (m, 2H), 2.07-1.98 (m, 2H), 1.96-1.89 (m, 1H), 1.76-1.69 (m, 1H), 1.64-1.55 (m, 2H), 0.94 (t, J=7.4 Hz, 3H). HRMS (ESI) m/z calcd for C₂₄H₃₂N₂O (M+H)⁺, 365.2587 found 365.2594.

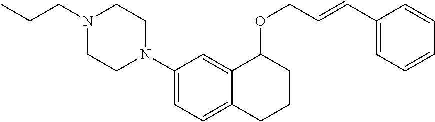 [SMILES: CCCN1CCN(c2ccc3c(c2)C(OC/C=C/c2ccccc2)CCC3)CC1]

1-(8-(Cinnamyloxy)-5,6,7,8-tetrahydronaphthalen-2-yl)-4-propylpiperazine (MDW-2-98). A solution of alcohol MDW-2-65 and 60% NaH dispersion in mineral oil (0.010 g, 0.25 mmol) in DMF (0.6 mL) was stirred at room temperature for 30 min and then cooled to 0° C. A solution of cinnamyl bromide (0.043 g, 0.22 mmol) in DMF (0.45 mL) was added and the solution was stirred at 0° C. for 30 min then allowed to warm to room temperature and stirred for 22 h. The reaction mixture was cooled to 0° C., quenched with 1 N aq. NaOH (2 mL), diluted with CH₂Cl₂ (20 mL), and washed with 1 N aq. NaOH (1×10 mL). The aqueous layer was extracted with CH₂Cl₂ (3×15 mL) and the combined organic extracts were dried (Na₂SO₄) and concentrated under reduced pressure. The crude residue was purified via flash chromatography (SiO₂) eluting with hexanes/EtOAc/Et₃N (89:10:1) affording 0.022 g (31%) of MDW-2-98 as a pale yellow oil. ¹H NMR (400 MHz, CDCl₃) δ 7.42-7.37 (comp, 2H), 7.35-7.29 (comp, 2H), 7.26-7.21 (m, 1H), 7.00 (d, J=8.4 Hz, 1H), 6.98 (d, J=2.7 Hz, 1H), 6.82 (dd, J=8.4, 2.7 Hz, 1H), 6.66 (d, J=16.0 Hz, 1H), 6.37 (dt, J=15.9, 5.9 Hz, 1H), 4.53-4.47 (m, 1H), 4.37-4.20 (comp, 2H), 3.22-3.15 (comp, 4H), 2.82-2.57 (comp, 6H), 2.41-2.33 (comp, 2H), 2.07-1.88 (comp, 3H), 1.78-1.67 (m, 1H), 1.63-1.50 (comp, 2H), 0.93 (t, J=7.4 Hz, 3H). HRMS (ESI) m/z calcd for C₂₆H₃₄N₂O (M+H)⁺, 391.2744 found 391.2754.

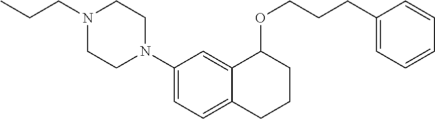 [SMILES: CCCN1CCN(c2ccc3c(c2)C(OCCCc2ccccc2)CCC3)CC1]

1-(8-(3-Phenylpropoxy)-5,6,7,8-tetrahydronaphthalen-2-yl)-4-propylpiperazine (MDW-2-126). A solution of alkene MDW-2-98 (0.0175 g, 0.0448 mmol) in EtOH (1.0 mL) was treated with 10% palladium on carbon (5 mg) and the resulting suspension was stirred under a hydrogen atmosphere (1 atm) at room temperature for 2 h. The mixture was filtered through Celite®, the filter cake was washed with CH₂C₂ (200 mL), and the filtrate was concentrated. The crude residue was purified via flash chromatography (SiO₂) eluting with hexanes/Et₂O/Et₃N (69:30:1) affording 0.0086 g (49%) of MDW-2-126 as a pale yellow oil. ¹H NMR (499 MHz, Chloroform-d) δ 7.30-7.25 (comp, 2H), 7.22-7.16 (comp, 3H), 6.99 (d, J=8.5 Hz, 1H), 6.97 (d, J=2.6 Hz, 1H), 6.83 (dd, J=8.5, 2.6 Hz, 1H), 4.38-4.33 (m, 1H), 3.69-3.62 (m, 1H), 3.54-3.48 (m, 1H), 3.21-3.13 (comp, 4H), 2.79-2.71 (comp, 3H), 2.67-2.56 (comp, 5 H), 2.39-2.32 (m, 2H), 2.03-1.88 (comp, 5H), 1.73-1.66 (m, 1H), 1.59-1.50 (m, 2H), 0.93 (t, J=7.4 Hz, 3H). HRMS (ESI) m/z calcd for C₂₆H₃₆N₂O (M+H)⁺, 393.2900 found 393.2903.

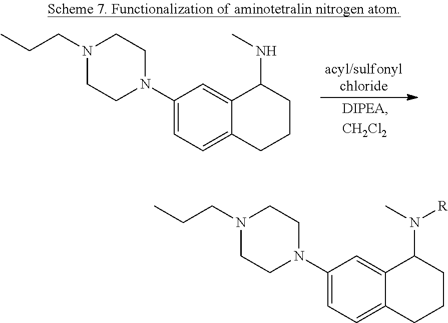

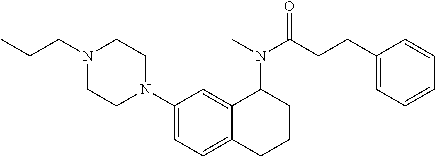 [SMILES: CCCN1CCN(c2ccc3c(c2)C(N(C)C(=O)CCc2ccccc2)CCC3)CC1]

N-Methyl-3-phenyl-N-(7-(4-propylpiperazin-1-yl)-1,2,3,4-tetrahydronaphthalen-1-yl)propanamide (MDW-1-238). 3-Phenylpropionyl chloride (0.01 g, 0.09 mL, 0.06 mmol) was added with stirring to a solution of amine MDW-2-74 (0.015 g, 0.052 mmol) and DIPEA (0.013 g, 0.018 mL, 0.104 mmol) in CH₂Cl₂ (0.5 mL) cooled to 0° C. The cooled solution was stirred for 5 min, then allowed to warm to room temperature and stirred for 2 h. The reaction mixture was diluted with CH₂Cl₂ (10 mL), washed with saturated aq. NH₄Cl (1×10 mL), dried (Na₂SO₄), and concentrated under reduced pressure. The crude residue was purified via flash chromatography (SiO₂) eluting with hexanes/EtOAc/Et₃N (59:40:1) affording 0.013 g (61%) of MDW-1-238 as a clear oil. ¹H NMR (499 MHz, CDCl₃) (rotamers) δ 7.33-7.17 (comp, 5H), 7.00 (m, 1H), 6.81-6.75 (m, 1H), 6.56-6.50 (m, 1H), 5.94-5.88 and 4.98-4.92 (m, 1H), 3.15-3.00 (comp, 6H), 2.89-2.52 (comp, 11H), 2.39-2.32 (comp, 2H), 2.01-1.65 (comp, 4H), 1.61-1.48 (comp, 2H), 0.96-0.90 (m, 3H). HRMS (ESI) m/z calcd for C₂₇H₃₇N₃O (M+H)⁺, 420.3009; found 420.3009.

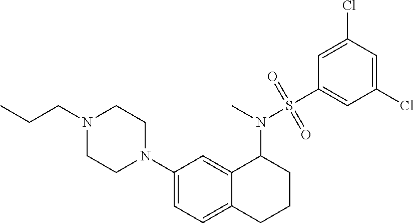 [SMILES: CCCN1CCN(c2ccc3c(c2)C(N(C)S(=O)(=O)c2cc(Cl)cc(Cl)c2)CCC3)CC1]

3,5-Dichloro-N-methyl-N-(7-(4-propylpiperazin-1-yl)-1,2,3,4-tetrahydronaphthalen-1-yl)benzenesulfonamide (MDW-1-237). 3,5-Dichlorobenzenesulfonyl chloride (0.014 g, 0.057 mmol) was added to a solution of amine MDW-2-74 (0.015 g, 0.052 mmol) and DIPEA (0.013 g, 0.018 mL, 0.104 mmol) in CH₂Cl₂ (0.5 mL) cooled to 0° C. The cooled solution was stirred for 5 min then allowed to warm to room temperature and stirred for an additional 2 h. The reaction mixture was diluted with CH₂Cl₂ (10 mL), washed with saturated aq. NH₄Cl (1×10 mL), dried (Na₂SO₄), and concentrated under reduced pressure. The crude residue was purified via flash chromatography (SiO₂) eluting with hexanes/EtOAc/Et₃N (89:10:1) affording 0.014 g (54%) of MDW-1-237 as a pale yellow oil. ¹H NMR (499 MHz, CDCl₃) δ 7.79 (d, J=1.9 Hz, 2H), 7.57 (t, J=1.9 Hz, 1H), 6.97 (d, J=8.4 Hz, 1H), 6.77 (dd, J=8.4, 2.4 Hz, 1H), 6.42 (d, J=2.3 Hz, 1H), 5.16-5.10 (m, 1H), 3.03-2.94 (m, 4H), 2.66-2.62 (comp, 5H), 2.59-2.54 (m, 4H), 2.39-2.34 (m, 2H), 1.95-1.88 (comp, 2H), 1.78-1.69 (comp, 2H), 1.60-1.51 (m, 2H), 0.94 (t, J=7.4 Hz, 3H). HRMS (ESI) m/z calcd for C₂₄H₃₁Cl₂N₃O₂S (M+H)⁺, 496.1587; found 496.1594.

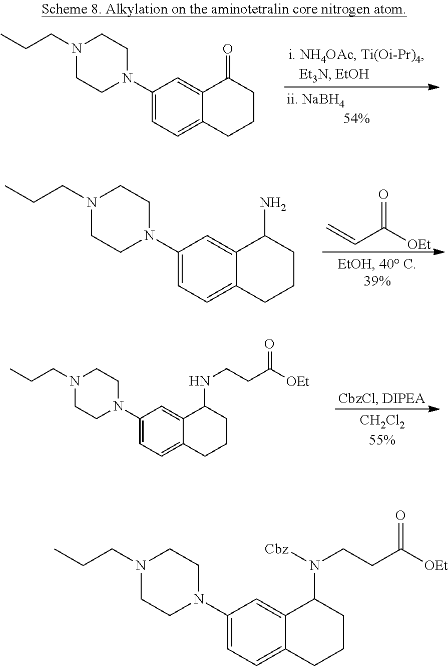

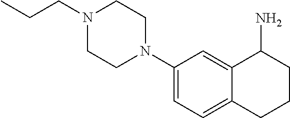 [SMILES: CCCN1CCN(c2ccc3c(c2)C(N)CCC3)CC1]

7-(4-Propylpiperazin-1-yl)-1,2,3,4-tetrahydronaphthalen-1-amine (MDW-2-100). In a modification of a literature procedure, tetralone MDW-2-57 (0.213 g, 0.782 mmol) was dissolved in EtOH (5.2 mL) in a resealable tube, whereupon Ti(OiPr)₄ (2.21 g, 2.3 mL, 7.82 mmol), Et₃N (0.39 g, 0.54 mL, 3.9 mmol) and NH₄OAc (0.301 g, 3.91 mmol) were sequentially added.² The tube was sealed, and the reaction was stirred at room temperature for 20 h, whereupon NH₄OAc (0.301 g, 3.91 mmol), Ti(OiPr)₄ (2.21 g, 2.3 mL, 7.82 mmol), and Et₃N (0.39 g, 0.54 mL, 3.9 mmol) were added. The tube was sealed, and the reaction was stirred at room temperature for 8 h. The solution was cooled to 0° C., and NaBH₄ (0.059 g, 1.6 mmol) was added in one portion. Stirring was continued at 0° C. for 1 h, and the mixture was added to 2 M aq. NH₄OH (10 mL). The suspension was filtered through a pad of Celite®, and the filter cake was washed with hot EtOAc (250 mL). The filtrate was concentrated under reduced pressure and partitioned between CH₂Cl₂ (15 mL) and saturated aq. NaHCO₃ (10 mL). The organic layer was separated and extracted with 1 M HCl (3×15 mL). The combined aqueous extracts were adjusted to pH 10 with 6 M NaOH and extracted with CH₂Cl₂ (3×50 mL). The combined organic extracts were dried (Na₂SO₄) and concentrated under reduced pressure. The crude residue was purified via flash chromatography (SiO₂) eluting with CH₂Cl₂/MeOH/Et₃N (94:5:1) affording 0.115 g (54%) of MDW-2-100 as a red oil. ¹H NMR (400 MHz, CDCl₃) δ 7.03 (d, J=2.6 Hz, 1H), 6.94 (d, J=8.4 Hz, 1H), 6.74 (dd, J=8.4, 2.6 Hz, 1H), 4.05-3.90 (comp, 3H), 3.20-3.12 (m, 4H), 2.75-2.50 (comp, 6H), 2.34-2.27 (m, 2H), 2.04-1.82 (comp, 2H), 1.77-1.65 (comp, 2H), 1.58-1.45 (m, 2H), 0.90 (t, J=7.4 Hz, 3H).

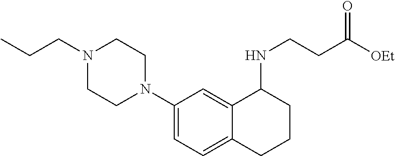 [SMILES: CCCN1CCN(c2ccc3c(c2)C(NCCC(=O)OCC)CCC3)CC1]

Ethyl 3-((7-(4-propylpiperazin-1-yl)-1,2,3,4-tetrahydronaphthalen-1-yl)amino)propanoate (MDW-2-104). Prepared from amine MDW-2-100 (0.0786 g, 0.287 mmol) and ethyl acrylate according to the general procedure for conjugate addition to ethyl acrylate. The crude residue was purified via flash chromatography (SiO₂) eluting with hexanes/EtOAc/Et₃N (54:45:1) affording 0.0416 g (39%) of MDW-2-104 as a pale yellow oil. ¹H NMR (499 MHz, CDCl₃) δ 6.99-6.94 (comp, 2H), 6.77 (dd, J=8.4, 2.7 Hz, 1H), 4.13 (q, J=7.1 Hz, 2H), 3.74 (t, J=5.1 Hz, 1H), 3.21-3.15 (comp, 4H), 3.05-2.88 (comp, 2H), 2.75-2.59 (comp, 6H), 2.54 (td, J=6.5, 2.7 Hz, 2H), 2.39-2.34 (comp, 2H), 1.97-1.79 (comp, 5H), 1.73-1.65 (m, 1H), 1.60-1.51 (comp, 2H), 1.25 (t, J=7.2 Hz, 3H), 0.93 (t, J=7.4 Hz, 3H).

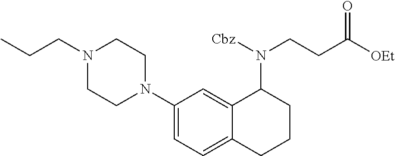 [SMILES: CCCN1CCN(c2ccc3c(c2)C(N(CCC(=O)OCC)C(=O)OCc2ccccc2)CCC3)CC1]

Ethyl 3-(((benzyloxy)carbonyl)(7-(4-propylpiperazin-1-yl)-1,2,3,4-tetrahydronaphthalen-1-yl)amino)propanoate (MDW-2-123). i-Pr₂NEt (0.029 g, 0.039 mL, 0.22 mmol) and CbzCl (0.023 g, 0.019 mL, 0.13 mmol) were added with stirring to a solution of amine MDW-2-104 (0.0416 g, 0.111 mmol) in CH₂Cl₂ (1.1 mL) cooled to 0° C. The solution was stirred at 0° C. for 22 h and then diluted with CH₂Cl₂ (10 mL). The solution was washed with 1 N HCl (2×10 mL), 1 N NaOH (2×10 mL), saturated aqueous NaHCO₃ (1×10 mL), dried (Na₂SO₄), and concentrated under reduced pressure. The crude residue was purified via flash chromatography (SiO₂) eluting with hexanes/EtOAc/Et₃N (74:25:1) affording 0.0310 g (55%) of MDW-2-123 as a clear oil. ¹H NMR (499 MHz, CDCl₃) rotamers δ 7.43-7.24 (comp, 5H), 7.00-6.94 (m, 1H), 6.79-6.73 (m, 1H), 6.58-6.53 (m, 1H), 5.47-5.05 (comp, 3H), 4.11-3.99 (comp, 2H), 3.54-3.40 (m, 1H), 3.24-3.00 (comp, 5H), 2.81-2.45 (comp, 8H), 2.38-2.31 (comp, 2H), 2.08-1.91 (comp, 2H), 1.81-1.67 (comp, 2H), 1.60-1.51 (comp, 2H), 1.23-1.16 (m, 3H), 0.93 (t, J=7.4 Hz, 3H). HRMS (ESI) m/z calcd for C₃₀H₄₁N₃O₄ (M+H)⁺, 508.3170; found 508.3181.

TABLE 5
Non-Sigma Receptor Binding of Aminotetralin Analogs.

| MDW-1- | MDW-1- | MDW-1- | MDW-1- | MDW-2- | MDW-1- | MDW-1- | MDW-1- | MDW-1- | MDW-1- | |
|---|---|---|---|---|---|---|---|---|---|---|
| 131 | 129 | 144 | 140 | 76 | 208 | 177 | 191 | 196 | 213 | Compound |
| | | | 367 | 156 | 90 | 528 | 569 | 3,805.00 | 335 | 5-HT1A |
| | | | | 362 | 175 | 261 | 441 | 1,307.00 | 317 | 5-HT1B |
| | | | 509 | 135 | 170 | 136 | 1,144.00 | 1,100.00 | 1,364.00 | 5-HT1D |
| | | | | | | | | | | 5-ht1e |
| | | | 547 | 315 | 446 | 632 | 1,732.00 | 1,277.00 | 957 | 5-HT2A |
| | | 3,590.00 | 263 | 100 | 158 | 99 | 771 | 2,144.00 | 692 | 5-HT2B |
| | | | 1,462.00 | 762 | 1,029.00 | | | | 2,631.00 | 5-HT2C |
| | | | | | | | | | | 5-HT3 |
| | | >10,000 | 2,501.00 | | | | 1,528.00 | | 1,879.00 | 5-ht5a |
| | | | 1,020.00 | 778 | 1,771.00 | 1,232.00 | 1,564.00 | 1,961.00 | 932 | 5-HT6 |
| | | 2,397.00 | 1,482.00 | 459 | 229 | 585 | 891 | 1,053.00 | 828 | 5-HT7 |
| | | | | 2,356.00 | 4,433.00 | | 1,408.00 | | | Alpha1A |
| | | | | | >10,000 | | | | | Alpha1B |
| | | | 1,805.00 | 2,595.00 | 6,625.00 | | | | | Alpha1D |
| | | | 1,114.00 | 1,031.00 | 2,983.00 | | | 980 | | Alpha2A |
| | | 524 | | 3,326.00 | >10,000 | | | | | Alpha2B |
| 2,545.00 | | | | | | | | | | Alpha2C |
| | | | | | | | | | | Beta1 |
| | | | | | | | | | | Beta2 |
| | | | | | | 8,039.00 | | | | Beta3 |
| | | | | | | | | | | BZP Rat Brain |
| | | | | | | | | | | Site |
| | | | 457.0 | 690.0 | 2,102.5 | 1,348.00 | | 1,921.00 | 4,049.00 | D1 |
| | | | (AVE) | (AVE) | (AVE) | | | | | |
| | | | | | | | 2,410.00 | 931 | | D2 |
| 3,393.00 | | | 462 | 438 | 537 | 597.0 | 315 | 177 | 852 | D3 |
| | | | | | | (AVE) | | | | |
| | | | | 6,660.00 | 2,968.00 | >10,000 | 1,718.00 | 2,509.00 | 319 | D4 |
| | | >10,000 | 2,597.00 | 3,964.00 | 4,389.00 | | | >10,000 | | D5 |
| | | | | | | | >10,000 | 1,914.00 | | DAT |
| | | | | | | | | | | DOR |
| | | | | | | | | | | GABAA |
| 182 | 118 | | 28 | 20 | 947 | — | | — | — | H1 |
| | | 327 | 211 | 302 | 1,852.00 | 291 | 1,289.00 | 1,039.00 | 333 | H2 |
| | | | 965 | 1,351.00 | | | | | | H3 |
| | | | | 5,875.3 | | | | | | H4 |
| | | | | (AVE) | | | | | | |
| | | 153.5 | 10,000.0 | | | | | | 5,353.00 | KOR |
| | | (AVE) | (AVE) | | | | | | | |
| | | | | | | | | | | M1 |
| | | | | | | | | | | M2 |
| | | | | | | | | | | M3 |
| | | | 2,983.00 | | | | | | 3,275.3 | M4 |
| | | | | | | | | | (AVE) | |
| | | | 3,691.00 | 6,621.00 | | | | | | M5 |
| | | 307 | | | | | | | | MOR |
| | | | | | | | | | | NET |
| 902 | | | 4,056.00 | 8,325.00 | 1,011.00 | | 1,669.6 | | 1,196.00 | PBR |
| | | | | | | | (AVE) | | | |
| | | | | | 8,702.00 | | | | >10,000 | SERT |
| | 223 | 1,268.00 | 86 | 200 | 1,649.00 | 474 | 2,364.00 | 199 | 2,101.00 | Sigma 1 |
| 522 | 1,174.00 | 50 | 2.1 | 5.5 | 4.5 | 21 | 19 | 9.4 | 7.2 | Sigma 2 |

Data represent Ki(nM) values obtained from non-linear regression of radioligand competition binding isotherms. Ki values are calculated from best fit IC₅₀ values using the Cheng-Prusoff equation. If <50% inhibition was observed in the 1° assay at 10 μM concentration, then a secondary assay to determine Ki was not performed.
Legend: Empty box is 1° assay <50%

### 2. Examples 4. Compounds being Synthesized

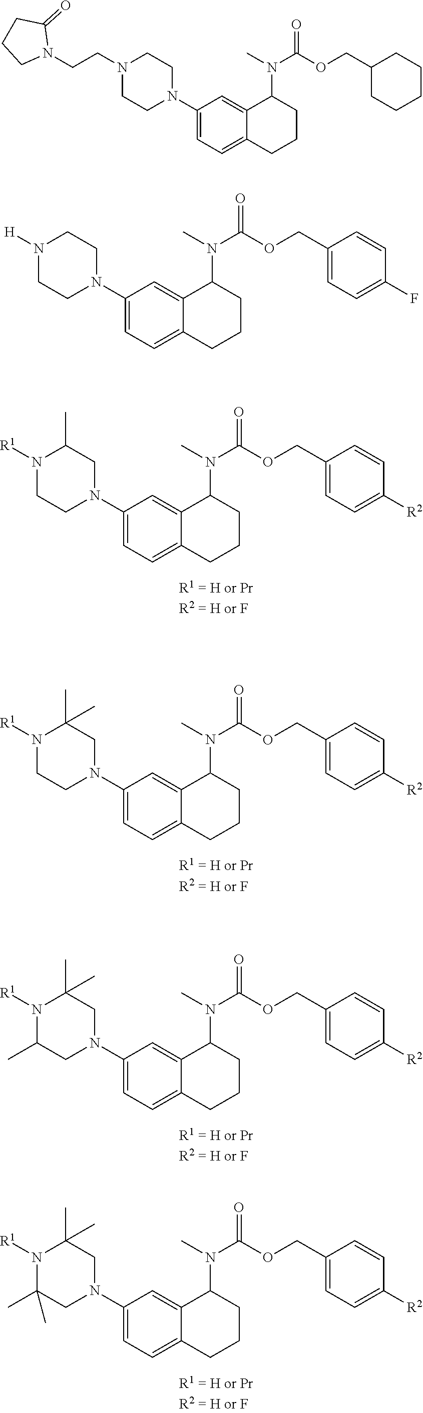

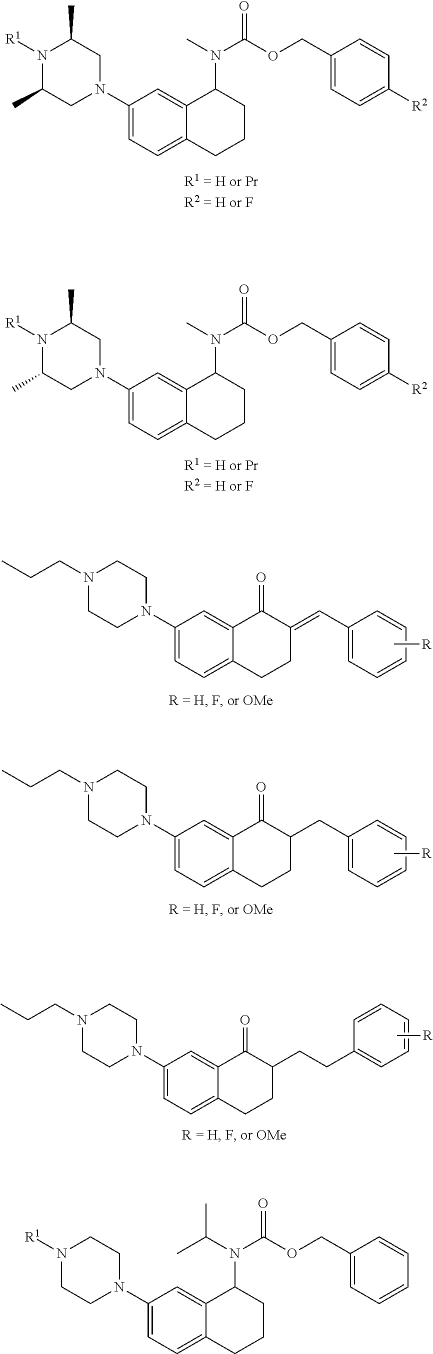

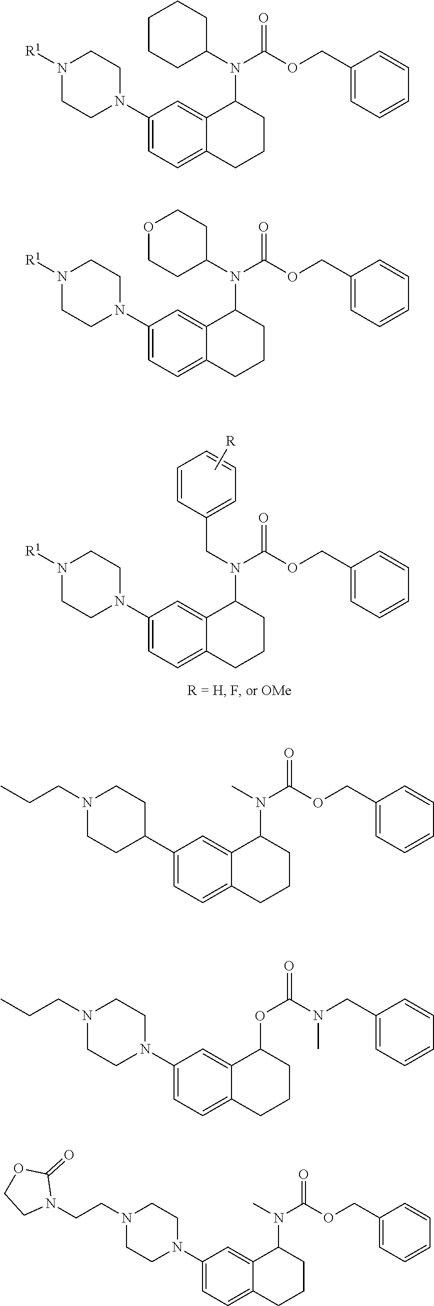

### 3. References

1. Hardy J, Selkoe D J. The amyloid hypothesis of Alzheimer's disease: progress and problems on the road to therapeutics. Science 2002; 297:353-356.
2. Backman L, Jones S, Berger A K, Laukka E J, Small B J. Multiple cognitive deficits during the transition to Alzheimer's diseases. J Intern Med. 2004; 256(3): 195-204.
3. Faizi M, Bader P L, Saw N, Nguyen T V, Beraki S, Wyss-Coray T, Longo F, Shamloo M. Thy1-hAPPLond/Swe+ mouse model of Alzheimer's disease displays broad behavioral deficits in sensorimotor, cognitive and social function. Brain Behav. 2012. 2(2): 142-154.
4. Rockenstein E, Mallory M, Mante M, Sisk A, Masliaha E. Early formation of mature amyloid-beta protein deposits in a mutant APP transgenic model depends on levels of Abeta(1-42). J. Neurosci. Res. 2001. 66(4): 573-582.

1. A compound having the formula: 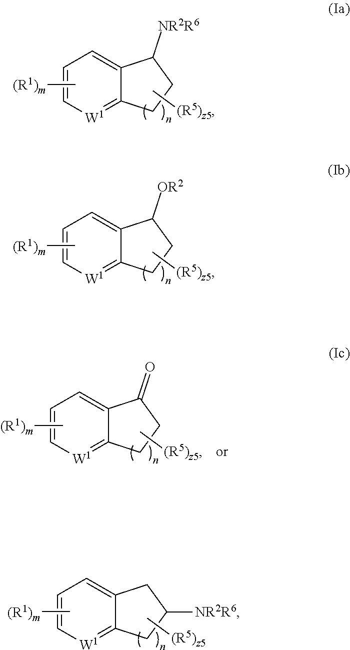
wherein:
R¹ is halogen, —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)₁R³, —S(O)ₙ₁OR³, —S(O)₁NR³R^{3A}, —NHNR³R^{3A}, —ONR³R^{3A}, —NHC(O)NHNR³R^{3A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups, or a group of the formula: —X₁—Y₁—Z₁—R⁷; wherein:
X₁ is a covalent bond, —O—, —S—, or —NX₁ₐ—, wherein X₁ₐ is hydrogen, alkyl, or substituted alkyl;
Y₁ is cycloalkylene, arylene, heteroarylene, heterocycloalkylene, or a substituted version of any of these groups;
Z₁ is alkylene, cycloalkylene, heteroalkylene, or a substituted version of either group;
R⁷ is hydrogen, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups, or a group of the formula: —N₃, —CF₃, —CCl₃, —CBr₃, —CI₃, —CN, —C(O)R^{7A}, —OR^{7A}, —NR^{7A}R^{7B}, —C(O)OR^{7B}, —C(O)NR^{7A}R^{7B}, —NO₂, —SR^{7A}, —S(O)ₙ₃R^{7A}, —S(O)ₙ₃OR^{7A}, —S(O)ₙ₃NR^{7A}R^{7B}, —NHNR^{7A}R^{7B}, —ONR^{7A}R^{7B}, —NOR^{7A}R^{7B}, or —NHC(O)NR^{7A}R^{3A};
R² is halogen, —N₃, —CF₃, —CCl₃, —CBr₃, —CI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙ₂NR⁴R^{4A}, —NHNR⁴R^{4A}, —ONR⁴R^{4A}, —NHC(O)NHNR⁴R^{4A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups;
n1, n2, and n3 are independently 1 or 2;
m is 1, 2, 3 or 4
n is 1, 2, 3 or 4; and
R³, R^{3A}, R⁴, R^{4A}, R^{7A}, R^{7B}, are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups;
R⁵ is halogen, —N₃, —CF₃, —CCl₃, —CBr₃, —CI₃, —CN, —C(O)R^{5C}, —OR^{5D}, NR^{5A}R^{5B}, —C(O)OR^{5D}, —C(O)NR^{5A}R^{5B}, —NO₂, —SR^{5D}, —S(O)ₙ₅R^{5C}, —S(O)ₙ₅OR^{5D}, —S(O)ₙ₅NR^{5A}R^{5B}, —NHNR^{5A}R^{5B}, —ONR^{5A}R^{5B}, —NHC(O)NHNR^{5A}R^{5B}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups;
n5 is independently 1 or 2;
z5 is independently and integer from 0 to 6;
R⁶ is halogen, —N₃, —CF₃, —CCl₃, —CBr₃, —CI₃, —CN, —C(O)R^{6C}, —OR^{6D}, NR^{6A}R^{6B}, —C(O)OR^{6D}, —C(O)NR^{6A}R^{6B}, —NO₂, —SR^{6D}, —S(O)ₙ₆R^{6C}, —S(O)ₙ₆OR^{6D}, —S(O)ₙ₆NR^{6A}R^{6B}, —NHNR^{6A}R^{6B}, —ONR^{6A}R^{6B}, —NHC(O)NHNR^{6A}R^{6B}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups;
n6 is independently 1 or 2;
W¹ is CH, C(R¹), or N; and
R^{5A}, R^{5B}, R^{5C}, R^{5D}, R^{6A}, R^{6B}, R^{6C}, and R^{6D} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups;
provided that when R¹ is N-methylpiperazinyl, m is 1, W¹ is CH, n is 2, then R² is not benzyl.

2. The compound of claim 1, having the formula: 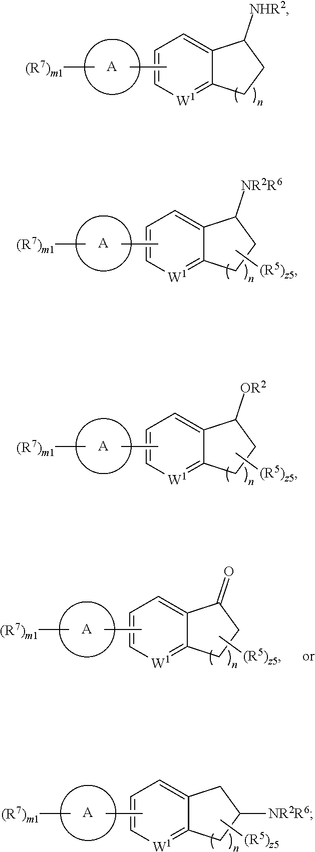
ring A is —X₁—Y₁—Z₁—; and
m1 is 0, 1, 2, 3, or 4.

3. The compound of claim 1 having the formula: 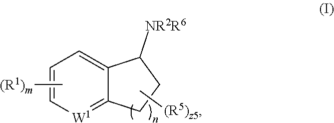
wherein:
R¹ is halogen, —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)₁R³, —S(O)ₙ₁OR³, —S(O)₁NR³R^{3A}, —NHNR³R^{3A}, —ONR³R^{3A}, —NHC(O)NHNR³R^{3A}, heterocycloalkyl, aryl, aralkyl, aralkenyl, heteroaryl, heteroaralkyl, heteroaralkenyl, or a substituted version of any of these groups, or a group of the formula: —X₁—Y₁—Z₁—R⁷; wherein:
X₁ is a covalent bond, —O—, —S—, or —NX₁ₐ—, wherein X₁ₐ is hydrogen, alkyl, or substituted alkyl;
Y₁ is cycloalkylene, arylene, heteroarylene, heterocycloalkylene, or a substituted version of any of these groups;
Z₁ is alkylene, cycloalkylene, heteroalkylene, or a substituted version of either group;
R⁷ is hydrogen, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups, or a group of the formula: —N₃, —CF₃, —CCl₃, —CBr₃, —CI₃, —CN, —C(O)R^{7A}, —OR^{7A}, —NR^{7A}R^{7B}, —C(O)OR^{7B}, —C(O)NR^{7A}R^{7B}, —NO₂, —SR^{7A}, —S(O)ₙ₃R^{7A}, —S(O)ₙ₃OR^{7A}, —S(O)ₙ₃NR^{7A}R^{7B}, —NHNR^{7A}R^{7B}, —ONR^{7A}R^{7B}, —NOR^{7A}R^{7B}, or —NHC(O)NR^{7A}R^{7B};
R² is halogen, —N₃, —CF₃, —CCl₃, —CBr₃, —CI₃, —CN, —C(O)R⁴, —OR⁴, —NR⁴R^{4A}, —C(O)OR⁴, —C(O)NR⁴R^{4A}, —NO₂, —SR⁴, —S(O)ₙ₂R⁴, —S(O)ₙ₂OR⁴, —S(O)ₙ₂NR⁴R^{4A}, —NHNR⁴R^{4A}, —ONR⁴R^{4A}, —NHC(O)NHNR⁴R^{4A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups;
n1 and n2 are independently 1 or 2;
m is 1, 2, 3 or 4
n is 1, 2, 3 or 4; and
R³, R^{3A}, R⁴, R^{4A} are independently hydrogen, oxo, halogen, —CF₃, —CN, —OH, —NH₂, —COOH, —CONH₂, —NO₂, —SH, —S(O)₂Cl, —S(O)₃H, —S(O)₄H, —S(O)₂NH₂, —NHNH₂, —ONH₂, —NHC(O)NHNH₂, —NHC(O) NH₂, —NHS(O)₂H, —NHC(O)H, —NHC(O)—OH, —NHOH, —OCF₃, —OCHF₂, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

4. The compound of claim 1, having the structure: 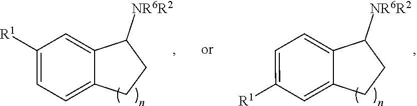
wherein R¹ is
halogen, —N₃, —CF₃, —CCl₃, —CBr₃, —CI₃, —CN, —C(O)R³, —OR³, —NR³R^{3A}, —C(O)OR³, —C(O)NR³R^{3A}, —NO₂, —SR³, —S(O)₁R³, —S(O)ₙ₁OR³, —S(O)₁NR³R^{3A}, —NHNR³R^{3A}, —ONR³R^{3A}, —NHC(O)NHNR³R^{3A}, heterocycloalkyl, aryl, aralkyl, aralkenyl, heteroaryl, heteroaralkyl, heteroaralkenyl, or a substituted version of any of these groups, or a group of the formula: —X₁—Y₁—Z₁—R⁷; wherein:
X₁ is a covalent bond, —O—, —S—, or —NX₁ₐ—, wherein X₁ₐ is hydrogen, alkyl, or substituted alkyl;
Y₁ is cycloalkylene, arylene, heteroarylene, heterocycloalkylene, or a substituted version of any of these groups;
Z₁ is alkylene, cycloalkylene, heteroalkylene, or a substituted version of either group;
R⁷ is hydrogen, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups, or a group of the formula: —N₃, —CF₃, —CCl₃, —CBr₃, —CI₃, —CN, —C(O)R^{7A}, —OR^{7A}, —NR^{7A}R^{7B}, —C(O)OR^{7B}, —C(O)NR^{7A}R^{7B}, —NO₂, —SR^{7A}, —S(O)ₙ₃R^{7A}, —S(O)ₙ₃OR^{7A}, —S(O)ₙ₃NR^{7A}R^{7B}, —NHNR^{7A}R^{7B}, —ONR^{7A}R^{7B}, —NOR^{7A}R^{7B}, or —NHC(O)NR^{7A}R^{7B}.

5. The compound of claim 1, wherein R¹ is halogen.

6. The compound of claim 1, wherein R² is halogen, —CN, —C(O)R⁴, —OR⁴, —NR⁴R^{4A}, —C(O)OR⁴, —C(O)NR⁴R^{4A}, —S(O)ₙ₂R⁴, —S(O)ₙ₂OR⁴, —S(O)ₙ₂NR⁴R^{4A}, —ONR⁴R^{4A}, —NHC(O)NHNR⁴R^{4A}, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups.

7. The compound of claim 1, wherein
R¹ is heterocycloalkyl, aryl, heteroaryl, or a substituted version of any of these groups, or a group of the formula: —X₁—Y₁—Z₁—R⁷; wherein:
X₁ is a covalent bond, —O—, —S— or —NX₁ₐ—, wherein X₁ₐ is hydrogen, alkyl, or substituted alkyl;
Y₁ is cycloalkylene, arylene, heteroarylene, heterocycloalkylene, or a substituted version of any of these groups;
Z₂ is alkylene, cycloalkylene, heteroalkylene, or a substituted version of either group;
R⁷ is hydrogen, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups, or a group of the formula: —N₃, —CF₃, —CCl₃, —CBr₃, —CI₃, —CN, —C(O)R^{7A}, —OR^{7A}, —NR^{7A}R^{7B}, —C(O)OR^{7B}, —C(O)NR^{7A}R^{7B}, —NO₂, —SR^{7A}, —S(O)ₙ₃R^{7A}, —S(O)ₙ₃OR^{7A}, —S(O)ₙ₃NR^{7A}R^{7B}, —NHNR^{7A}R^{7B}, —ONR^{7A}R^{7B}, —NOR^{7A}R^{7B}, or —NHC(O)NR^{7A}R^{7B}.

8. The compound of claim 1, wherein R¹ is heterocycloalkyl, substituted heterocycloalkyl, or a group of the formula: —X₁—Y₁—Z₁—R⁷; wherein:
X₁ is a covalent bond, —O—, —S— or —NX₁ₐ—, wherein X₁ₐ is hydrogen, alkyl, or substituted alkyl;
Y₁ is cycloalkylene, arylene, heteroarylene, heterocycloalkylene, or a substituted version of any of these groups;
Z₁ is alkylene, cycloalkylene, heteroalkylene, or a substituted version of either group;
R⁷ is hydrogen, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups, or a group of the formula: —N₃, —CF₃, —CCl₃, —CBr₃, —CI₃, —CN, —C(O)R^{7A}, —OR^{7A}, —NR^{7A}R^{7B}, —C(O)OR^{7B}, —C(O)NR^{7A}R^{7B}, —NO₂, —SR^{7A}, —S(O)ₙ₃R^{7A}, —S(O)ₙ₃OR^{7A}, —S(O)ₙ₃NR^{7A}R^{7B}, —NHNR^{7A}R^{7B}, —ONR^{7A}R^{7B}, —NOR^{7A}R^{7B}, or —NHC(O)NR^{7A}R^{7B}.

9. The compound of claim 1, wherein R¹ is a group of the formula: —X₁—Y₁—Z₁—R⁷; wherein:
X₁ is a covalent bond, —O—, —S— or —NX₁ₐ—, wherein X₁ₐ is hydrogen, alkyl, or substituted alkyl;
Y₁ is arylene, heterocycloalkylene, or a substituted version of either groups;
Z₁ is alkylene, cycloalkylene, heteroalkylene, or a substituted version of either group;
R⁷ is hydrogen, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups, or a group of the formula: —N₃, —CF₃, —CCl₃, —CBr₃, —CI₃, —CN, —C(O)R^{7A}, —OR^{7A}, —NR^{7A}R^{7B}, —C(O)OR^{7B}, —C(O)NR^{7A}R^{7B}, —NO₂, —SR^{7A}, —S(O)ₙ₃R^{7A}, —S(O)ₙ₃OR^{7A}, —S(O)ₙ₃NR^{7A}R^{7B}, —NHNR^{7A}R^{7B}, —ONR^{7A}R^{7B}, —NOR^{7A}R^{7B}, or —NHC(O)NR^{7A}R^{7B}.

10. The compound of claim 1, having the formula: 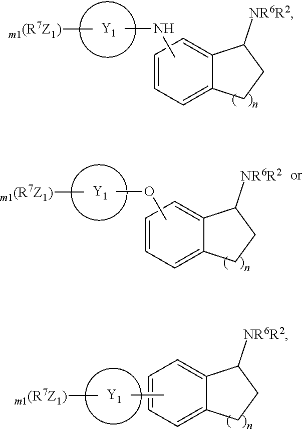
wherein
Z₁ is alkylene, cycloalkylene, heteroalkylene, or a substituted version of either group;
R⁷ is hydrogen, alkyl, cycloalkyl, alkenyl, cycloalkenyl, alkynyl, heteroalkyl, heterocycloalkyl, aryl, aralkyl, aralkenyl, heteroaryl, heteroaralkyl, heteroaralkenyl, or a substituted version of any of these groups, or a group of the formula: —N₃, —CF₃, —CCl₃, —CBr₃, —CI₃, —CN, —C(O)R^{7A}, —OR^{7A}, —NR^{7A}R^{7B}, —C(O)OR^{7B}, —C(O)NR^{7A}R^{7B}, —NO₂, —SR^{7A}, —S(O)ₙ₃R^{7A}, —S(O)ₙ₃OR^{7A}, —S(O)ₙ₃NR^{7A}R^{7B}, —NHNR^{7A}R^{7B}, —ONR^{7A}R^{7B}, —NOR^{7A}R^{7B}, or —NHC(O)NR^{7A}R^{7B}
Y₁ is arylene, heteroarylene, cycloalkylene or heterocycloalkylene; and
m1 is 0, 1, 2, 3, or 4.

11.-17. (canceled)

18. The compound of claim 1, wherein n is 1 or 2.

18.5. (canceled)

19. The compound of claim 1, wherein R² is —OR⁴, —NR⁴R^{4A}, —C(O)OR⁴, alkyl, cycloalkyl, alkenyl, cycloalkenyl, alkynyl, heterocycloalkyl, aryl, aralkyl, aralkenyl, heteroaryl, heteroaralkyl, heteroaralkenyl, or a substituted version of any of these groups.

20. The compound of claim 19, wherein R² is —OR⁴, —NR⁴R^{4A}, —C(O)OR⁴, alkyl, alkenyl, alkynyl, heterocycloalkyl, aryl, aralkyl, heteroaryl, heteroaralkyl, or a substituted version of any of these groups.

21.-25. (canceled)

26. The compound of claim 1, having the formula:

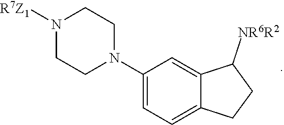

27. The compound of claim 1 of the formula:

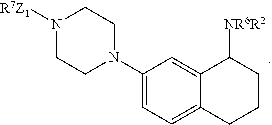

28. The compound of claim 27, wherein Z₁ is substituted or unsubstituted alkylene or cycloalkylene.

29.-30. (canceled)

31. The compound of claim 26, wherein R² is —C(O)OR⁴, wherein R⁴ is aralkyl or substituted aralkyl.

32.-33. (canceled)

34. The compound of claim 31, wherein R⁴ is benzyl.

35. A pharmaceutical composition comprising a compound of claim 1 and a pharmaceutically acceptable excipient or pharmaceutically acceptable salt.

36. A method of treating;
(a) cancer;
(b) a neurodegenerative disease;
(c) ethanol withdrawal;
(d) anxiety or depression;
(e) neuropathic pain; or
(f) traumatic brain injury;
in a subject in need thereof, the method comprising administering an effective amount of a compound of claim 1.

37.-57. (canceled)
